(19) United States
(12) Patent Application Publication
(10) Pub. No.: US 2024/0247075 A1
(43) Pub. Date: Jul. 25, 2024

(54) MULTISPECIFIC ANTIBODIES

(71) Applicant: Genentech, Inc., South San Francisco, CA (US)

(72) Inventors: Germaine FUH, Pacifica, CA (US); Jenny M. Bostrom, South San Francisco, CA (US)

(21) Appl. No.: 18/505,660

(22) Filed: Nov. 9, 2023

Related U.S. Application Data
(62) Division of application No. 17/232,537, filed on Apr. 16, 2021, now Pat. No. 11,851,501.
Application No. 17/232,537 is a division of application No. 16/133,536, filed on Sep. 17, 2018, now Pat. No. 11,008,401.
Application No. 16/133,536 is a division of application No. 11/893,693, filed on Aug. 17, 2007, now Pat. No. 10,118,970.
(60) Provisional application No. 60/937,814, filed on Jun. 28, 2007.
(60) Provisional application No. 60/841,350, filed on Aug. 30, 2006.

(51) Int. Cl. C07K 16/32; C07K 16/22; C07K 16/28; C07K 16/42; C07K 16/46

(52) U.S. Cl. CPC C07K 16/32; C07K 16/22; C07K 16/2878; C07K 16/4283; C07K 16/468; C07K 2317/55; C07K 2317/56; C07K 2317/622; C07K 2317/73

(57) ABSTRACT

The invention provides multispecific antibodies and methods of making and using such antibodies.

### CROSS-REFERENCE TO RELATED APPLICATIONS

This application is a divisional application of U.S. application Ser. No. 17/232,537, filed Apr. 16, 2021, which is a divisional of U.S. application Ser. No. 16/133,536, filed Sep. 17, 2018, now U.S. Pat. No. 11,008,401, which is a divisional of U.S. application Ser. No. 11/893,693, filed Aug. 17, 2007, now U.S. Pat. No. 10,118,970, which claims the benefit of U.S. Application Ser. No. 60/937,814, filed Jun. 28, 2007, and U.S. Application Ser. No. 60/841,350, filed Aug. 30, 2006, the disclosures of which are hereby incorporated by reference.

### SEQUENCE LISTING

The instant application contains a Sequence Listing which has been submitted electronically in XML format and is hereby incorporated by reference in its entirety. Said XML copy, created on Nov. 8, 2023, is named 50474-015006_Sequence_Listing_11_8_23.xml and is 380,369 bytes in size.

### FIELD OF THE INVENTION

The present invention relates to multispecific antibodies, and methods of making and using such antibodies.

### BACKGROUND OF THE INVENTION

Antibodies are specific immunoglobulin polypeptides produced by the vertebrate immune system in response to challenge by foreign proteins, glycoproteins, cells, or other antigenic foreign substances. An important part of this process is the generation of antibodies that bind specifically to a particular foreign substance. The binding specificity of such polypeptides to a particular antigen is highly refined, and the multitude of specificities capable of being generated by the individual vertebrate is remarkable in its complexity and variability. Thousands of antigens are capable of eliciting responses, each almost exclusively directed to the particular antigen which elicited it.

Specific antigen recognition is essential for antibodies to function in the adaptive immune response. The combinatorial association of heavy chain (HC) and light chain (LC) is conserved in all vertebrates in the generation of the antibody repertoire. There is, however, asymmetry of diversity in the two chains. The variable domain of HC (V_{H}) contains significantly higher sequence diversity and contributes the determinants of antigen recognition more often than the variable domain of the LC (V_{L}). The role of the LC in determining antigen-specificity is indicated by a process called receptor editing. Ongoing recombination of the V_{L} genes to edit the B cell receptor is the main mechanism to correct self reactive antibody precursors, which appear to constitute a significant portion of the initial repertoire (˜75%). Altering of the light chain is demonstrated to extinguish unwanted binding specificity or multi-specificity.

The specificity of antibodies and antibody fragments for a particular antigen or antigens makes antibodies desirable therapeutic agents. Antibodies and antibody fragments can be used to target particular tissues, for example, a tumor, and thereby minimize the potential side effects of non-specific targeting. As such, there is a current and continuing need to identify and characterize therapeutic antibodies, especially antibodies, fragments, and derivatives thereof, useful in the treatment of cancer and other proliferative disorders.

### SUMMARY OF THE INVENTION

The present invention provides a method of making a multispecific antibody comprising the steps of (1) diversifying the amino acid sequence of a light chain variable domain (V_{L}) of an antibody, wherein prior to the diversification, the antibody comprised a V_{L} and a heavy chain variable domain (V_{H}) capable of binding to a first epitope and (2) selecting a diversified antibody capable of binding to the first epitope and a second epitope. According to one embodiment, prior to diversification, the antibody in step 1 is monospecific. According to another embodiment, the V_{L}/V_{H} of the antibody after using the methods of this invention, is bispecific. In one embodiment, the V_{L} amino acid sequence is diversified by altering the nucleic acid sequence encoding the V_{L} and expressing the diversified V_{L}. In a further embodiment, the diversification is by altering a plurality of predetermined amino acid residue positions. According to one embodiment, the residues are diversified based on the diversity of a plurality of naturally occurring light chain amino acid sequences. Additionally or alternatively, the residues are diversified based on the accessibility to solvent. According to one embodiment, the diversified V_{L} are displayed on phage with the V_{H} during the selection step. According to a further embodiment, the binding affinity of the multispecific antibody can be improved by subjecting the antibody to affinity maturation (e.g., homolog scanning or other mutations of the V_{L} and/or V_{H}).

According to one embodiment the residue positions diversified are selected from the group consisting of: (1) positions 28, 30-32, 66-70, 92-93, optionally at positions 30a-e and 93(a)-(b); (2) positions 28, 30-32, 66-70, 92-93, optionally at positions 30a-f; (3) positions 28-32, 50-53, 92-93, optionally at positions 30a-f and 93a-b; (4) positions 28, 30-32, 50-53, 92-93, optionally at positions 30a-f and 93a-b; (5) positions 28-32, 50-53, 92-93; (6) positions 28-32, 50-53, 92-93, optionally at 30a-b (7) positions 28-32, 50-53, 91-93, 94, optionally at 30a-f and 93a-b; and (8) positions 29-32, 50-53, 66-67, 69-70, 92-93, 94, optionally at 30a-f. According to one preferred embodiment, the V_{L} residues are diversified according to any one of the library designs described in FIG. 1.

According to one embodiment, the selection step for binding to the first epitope or the selection for binding to the second epitope occurs in the presence of both diversified V_{L} and V_{H}. According to another embodiment, the diversified V_{L} and V_{H} can bind the first epitope and second epitope simultaneously. In an alternative embodiment, the diversified V_{L}, and V_{H} bind the first epitope and second epitope mutually exclusively. In one embodiment, the first epitope is from one biological molecule and the second epitope is from the same biological molecule. In another embodiment, the first epitope is from one biological molecule and the second epitope is from a second biological molecule. According to one embodiment the first biological molecule and the second biological molecule are structurally unsimilar.

According to one embodiment, the first biological molecule and second biological molecule pairs are selected from the following group of pairs: VEGF/HER2, VEGF-A/HER2, HER2/DR5, VEGF-A/PDGF, HER1/HER2, CD20/BR3, VEGF-A/VEGF-C, VEGF-C/VEGF-D, TNFalpha/TGF-beta, TNFalpha/IL-2, TNF alpha/IL-3, TNFalpha/IL-4, TNFalpha/IL-5, TNFalpha/IL6, TNFalpha/IL8, TNFalpha/IL-9. TNFalpha/IL-10, TNFalpha/IL-11. TNFalpha/IL-12, TNFalpha/IL-13, TNFalpha/IL-14. TNFalpha/IL-15, TNFalpha/IL-16, TNFalpha/IL-17, TNFalpha/IL-18, TNFalpha/IL-19, TNFalpha/IL-20, TNFalpha/IFNalpha, TNFalpha/CD4, VEGF/IL-8, VEGF/MET, VEGFR/MET receptor, HER2/Fc, HER2/HER3; EGFR/HER2, EGFR/HER3, EGFR/HER4, TNFalpha/IL-3, TNFalpha/IL-4, IL-13/CD40L, IL4/CD40L, TNFalpha/ICAM-1, TNFR1/IL-1R, TNFR1/IL-6R and TNFR1/IL-18R. In particular embodiments, the first biological molecule and second biological molecule are VEGF/HER2, HER2/DR5, or HER2/Fc.

According to one embodiment, one of the biological molecules is a molecule that can increase the half-life of the multispecific antibody when bound to the antibody in vivo. In another embodiment, the biological molecule is serum albumin or the neonatal Fc receptor (FcRn).

According to another embodiment, one of the biological molecules is a molecule that can increase the effector function of a multispecific antibody when bound to the antibody in vivo. In one embodiment, the biological molecule binds to a cell surface protein on natural killer cells or macrophages. The cell surface protein may be C1q or an Fc receptor.

The present invention also provides multispecific antibodies made by the methods of this invention. It is contemplated that a multispecific antibody of this invention can comprise one or a plurality of the V_{L}/V_{H} domains created by the methods of this invention alone or in combination with the hypervariable domains of antibodies not created by the methods of this invention. A multispecific antibody of this invention can exist in a variety of forms (e.g., antibody fragments, multimeric, etc). According to one embodiment, the multispecific antibody is any one of the antibodies of FIGS. 5-10, 13-16, FIG. 34, FIG. 39, and Table 2. In a particular embodiment, the multispecific antibody is a bH1 antibody of FIG. 34 or a fragment thereof.

The multispecific antibodies of this invention can be used in a variety of ways, e.g., in therapies to treat illnesses or diseases, in research or in purification. In one embodiment, the invention provides a method of treating a tumor (e.g., a cancerous tumor) in a subject. This method involves administering to the subject the multispecific antibody of any one of the antibodies of FIGS. 5-10, 13-16, FIG. 34, FIG. 39, and Table 2, a fragment thereof, or an antibody made by the methods of the invention, where the administering is for a time and in an amount sufficient to treat or prevent the tumor in the subject. In a particular embodiment, the multispecific antibody is a bH1 antibody of FIG. 34 or a fragment thereof.

In another aspect, the invention features a method of treating a subject at risk of developing a tumor. The method of the invention are also advantageous in reducing or preventing the recurrence of a tumor, for example, a dormant tumor that persists after removal of the primary tumor, or in reducing or preventing the occurrence or proliferation of metastatic tumors. This method involves administering to the subject the multispecific antibody of any one of the antibodies of FIGS. 5-10, 13-16, FIG. 34, FIG. 39, and Table 2, a fragment thereof, or an antibody made by the methods of the invention.

In a further aspect, the invention features a method of treating a subject having or being at risk of developing an autoimmune disease. This method involves administering to the subject the multispecific antibody of any one of the antibodies of FIGS. 5-10, 13-16, FIG. 34, FIG. 39, and Table 2, a fragment thereof, or an antibody made by the methods of the invention, where the administering is for a time and in an amount sufficient to treat or prevent the autoimmune disease in the subject.

Also, contemplated are kits, compositions, and articles of manufacture comprising the multispecific antibodies.

### BRIEF DESCRIPTION OF THE DRAWINGS

FIG. 1 shows the designed diversity in various LC libraries.

FIG. 2 shows a summary of four light chain libraries used to alter anti-VEGF antibodies or anti-Her2 antibodies to bind to an additional target. The italicized NNK and XYZ refer to codon sets. Ys, Ds, Ts and Ss refer to soft randomizations by having tyrosine, aspartic acid, threonine and serine, respectively, occurring 50% of the time and any one of the 20 amino acids occurring the other 50% of the time. D/Ds and T/Ts refer to a soft randomization having D or T, respectively, occurring 75% of the time and any one of the 20 amino acids occurring the other 25% of the time.

FIG. 3 shows sequences of HC, LC CDR residues of light chain templates (SEQ ID NOS:1-6 and 53-63).

FIG. 4 shows sorting conditions and enrichment of Library C and D.

FIG. 5 shows VEGF binders (SEQ ID NOS:64-105). Residues 28, 30, 30a, 31, 92, 93, and 93a were fully diverse. Residues 32, 50, 53, 91 and 94 were restricted. Residues 29, 33, and 51 were limited (<3).

FIG. 6 shows DR5 binders (SEQ ID NOS: 106-216).

FIG. 7 shows human VEGF binders, combined plate and solution selection (SEQ ID NOS:4-6 and 217-231).

FIGS. 8A and 8B show clones that bind both VEGF and HER2 (SEQ ID NOS:232-421).

FIG. 9 shows clones that only bind VEGF and lost the binding activity with HER2 (SEQ ID NOS:421-499).

FIG. 10 shows DR5 binders plate sorted directly on HER2 (SEQ ID NOS:4-6 and 500-508).

FIG. 11 shows clones binding to VEGF.

FIGS. 12A and 12B show clones that block VEGF binding to VEGFR1-D2 or D1.

FIGS. 13A and 13B show the affinities of VEGF binders (SEQ ID NOS:4-6 and 509-532) from library L1/L2/L3-C,D.

FIG. 14 shows a summary of specific binders from LC library (SEQ ID NOS:4-6 and 533-547).

FIG. 15 shows clones that can bind both (FIG. 15A) hVEGF and HER2 or (FIG. 15B) DR5 and HER2 (SEQ ID NOS:4-6 and 548-563).

FIG. 16 shows the LC library binders used in scFv′2 formation and displayed on phage (SEQ ID NOS:4-6 and 564-590).

FIG. 17 shows the expression of various clones in Fab or hIgG form.

FIGS. 18A and 18B show ELISAs of clones in hIgG form binding to hVEGF165.

FIG. 19 shows ELISAs of clones in hIgG form binding to immobilized protein targets.

FIG. 20 shows competitive ELISAs of clones in hIgG form in the presence of Her2 and VEGF or DR5.

FIG. 21 shows a Biacore Analysis of binding to VEGF or HER2.

FIG. 22 shows binding to HER2-ECD or hVEGF with an IgG or Fab having a light chain obtained from a different binding clone.

FIGS. 23A and 23B show an anti-VEGF antibody blocking VEGF interaction with VEGFR1 D 1-3 and KDR D1-7.

FIG. 24 shows antibodies blocking B20-4.1 and VEGF binding.

FIG. 25 shows antibodies blocking Avastin® antibody and VEGF binding.

FIG. 26 shows the inhibition of VEGF induced HUVEC cell proliferation with anti-VEGF antibodies.

FIG. 27 shows binding of bispecific antibodies to HER2 expressed on NR6 cells.

FIG. 28 shows codons of H1 that were shotgun scanned.

FIG. 29 shows a library consortium (SEQ ID NOS:591-602).

FIG. 30 shows an antibody clone with shotgun scan mutations screened by binding to VEGF.

FIG. 31 shows an antibody clone with shotgun scan mutations screened by binding to HER2.

FIG. 32 shows the results of an alanine scan of bH1 for (FIG. 32A) VEGF binding or (FIG. 32B) HER2 binding and the results of a homolog scan of bH1 for (FIG. 32C) VEGF binding or (FIG. 32D) HER2 binding.

FIG. 33 shows alanine scanning results of bH1 or the Herceptin® antibody mutants.

FIG. 34 shows bH1 VEGF-affinity matured clone sequences and binding affinity for VEGF or HER2 (SEQ ID NOS:603-641).

FIGS. 35A through 35D show crystal structures of the bispecific bH1 Fab bound to HER2 or VEGF.

FIG. 35E is a graph showing that anti-VEGF antibodies block hVEGF binding to VEGF receptor 2 (VEGFR2).

FIG. 35F is a series of pie charts showing the individual CDR contributions to the structural paratope for bH1. The paratope size for VEGF is 730 Å² and for HER2 is 690 Å². The heavy chain CDRs are indicated in gray and the light chain CDRs in white.

FIGS. 36A through 36C show the energetically important binding sites of bH1 for VEGF and HER2 binding.

FIGS. 37A and 37B show shotgun alanine- and homolog scanning of bH1 Fab for binding to VEGF and HER2.

FIG. 38 shows the natural and designed diversity of light chain CDRs. At each position, the Herceptin® antibody sequence is shown in parenthesis. An “*” denotes an insertion not present in the Herceptin® antibody.

FIGS. 39A and 39B show the sequences of specific antigen-binding clones isolated from the light chain (LC) library. FIG. 39A shows the LC CDR sequences of monospecific phage clones biding to VEGF, DR5, and Fc (SEQ ID NOS:4-6 and 642-795), and FIG. 39B shows bispecific Fabs binding to VEGF/HER2, DR5/HER2, and Fc/HER2 (SEQ ID NOS:4-6 and 796-897). The light chain framework and heavy chain sequences correspond to that of the Herceptin® antibody with the exception of LC framework substitution R66G.

FIG. 40 is a graph showing binding specificity of the antibodies derived from the LC library. The results for antibodies bH1, bH3, 3-1, bD1, bD2, 4-1, and 4-5 are shown. Bound IgG antibodies were detected spectrophotometrically (optical density at 450 nm, y-axis). The proteins included in the assay were (left to right for each antibody) human vascular endothelial growth factor A (hVEGF-A), hVEGF-C, hVEGF-D, hHER2 extracellular domain (ECD), epidermal growth factor receptor extracellular domain (hEGFR), human death receptor 5 (hDR5), bovine serum albumin (BSA), casein, fetal bovine serum (FBS), WIL2 cell lysate, and NR6 cell lysate.

FIG. 41 shows results from affinity maturation experiments indicating that the libraries described herein can be used to improve the affinity for both VEGF and HER2.

FIG. 42 shows the results of competitive binding experiments for bH1 to VEGF or HER2.

FIGS. 43A and 43B show that bH1 IgG inhibits HER2 and VEGF-mediated cell proliferation.

### DETAILED DESCRIPTION OF THE INVENTION

The present invention provides methods of making multispecific antibodies and antibody fragments, as well as antibodies identified using these methods and their use. In general, the methods of the invention involve diversifying the light chain variable domain or the heavy chain variable domain of an antibody to generate variants that can be stably expressed in a library. Diversified antibodies that are capable of specifically binding two epitopes are then selected from this library and further characterized.

Exemplary antibodies identified using the methods of the invention include antibodies that bind both HER2 (human epithelial growth factor receptor 2) and VEGF (vascular endothelial growth factor), as well as antibodies that bind both HER2 and DR5 (death receptor 5). In particular, the data described herein, for instance, in the below Examples, show that mutations in the light chain complementarity determining regions (CDRs) of a HER2 antibody confer dual binding capabilities for unrelated protein antigens as well as HER2. One bi-specific high affinity HER2/VEGF antibody is extensively characterized. In addition, the crystal structures of this bi-specific Fab in complex with HER2 and VEGF are shown and the energetic contribution of the Fab residues by mutagenesis is evaluated. The binding sites for the two antigens overlap extensively; most of the CDR residues that contact HER2 also engage VEGF. Energetically, however, the residues of the heavy chain dominate the HER2 specificity while the light chain dominates VEGF specificity.

The HER2/VEGF bi-specific antibody inhibits both HER2 and VEGF-mediated cell proliferation. These results demonstrate that altering the sequence of the light chain variable domain of an antibody can generate antibodies with dual specificity and function. For example, bH1 has the potential to target two mechanisms of tumor progression: tumor cell proliferation mediated by HER2 and tumor angiogenesis mediated by VEGF.

### I. Definitions

The term “multispecific antibody” is used in the broadest sense and specifically covers an antibody comprising a heavy chain variable domain (V_{H}) and a light chain variable domain (V_{L}), where the V_{H}V_{L} unit has polyepitopic specificity (i.e., is capable of binding to two different epitopes on one biological molecule or each epitope on a different biological molecule). Such multispecific antibodies include, but are not limited to, full length antibodies, antibodies having two or more V_{L} and V_{H} domains, antibody fragments such as Fab, Fv, dsFv, scFv, diabodies, bispecific diabodies and triabodies, antibody fragments that have been linked covalently or non-covalently. “Polyepitopic specificity” refers to the ability to specifically bind to two or more different epitopes on the same or different target(s). “Monospecific” refers to the ability to bind only one epitope. According to one embodiment the multispecific antibody in an IgG1 form binds to each epitope with an affinity of 5 μM to 0.001 pM, 3 μM to 0.001 pM, 1 μM to 0.001 pM, 0.5 μM to 0.001 pM or 0.1 μM to 0.001 pM.

The basic 4-chain antibody unit is a heterotetrameric glycoprotein composed of two identical light (L) chains and two identical heavy (H) chains (an IgM antibody consists of 5 of the basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has, at the N-terminus, a variable domain (V_{H}) followed by three constant domains (C_{H}) for each of the α and γ chains and four C_{H} domains for u and & isotypes. Each L chain has, at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, C T, 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA. IgD, IgE, IgG, and IgM, having heavy chains designated α, δ, γ, ε, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about residues 26-35 (H1), 50-65 (H2) and 95-102 (H3) in the V_{H} (in one embodiment, H1 is around about residues 31-35); Kabat et al., Sequences of Proteins of Immunological Interest, 5th Ed. Public Health Service, National Institutes of Health, Bethesda, MD. (1991)) and/or those residues from a “hypervariable loop” (e.g., residues 26-32 (L1), 50-52 (L2), and 91-96 (L3) in the V_{L}, and 26-32 (H1), 53-55 (H2), and 96-101 (H3) in the V_{H}; Chothia and Lesk, J. Mol. Biol. 196:901-917 (1987)).

The term “monoclonal antibody” as used herein refers to an antibody from a population of substantially homogeneous antibodies, i.e., the individual antibodies comprising the population are substantially similar and bind the same epitope(s), except for possible variants that may arise during production of the monoclonal antibody, such variants generally being present in minor amounts. Such monoclonal antibody typically includes an antibody comprising a variable region that binds a target, wherein the antibody was obtained by a process that includes the selection of the antibody from a plurality of antibodies. For example, the selection process can be the selection of a unique clone from a plurality of clones, such as a pool of hybridoma clones, phage clones or recombinant DNA clones. It should be understood that the selected antibody can be further altered, for example, to improve affinity for the target, to humanize the antibody, to improve its production in cell culture, to reduce its immunogenicity in vivo, to create a multispecific antibody, etc., and that an antibody comprising the altered variable region sequence is also a monoclonal antibody of this invention. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5): 1073-1093 (2004); Fellouse, Proc. Nat. Acad. Sci. USA 101(34):12467-12472 (2004); and Lee et al. J. Immunol. Methods 284(1-2):119-132 (2004) and technologies for producing human or human-like antibodies from animals that have parts or all of the human immunoglobulin loci or genes encoding human immunoglobulin sequences (see, e.g., WO98/24893, WO/9634096, WO/9633735, and WO/91 10741, Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); 5,545,807; WO 97/17852, U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995).

An “intact” antibody is one which comprises an antigen-binding site as well as a C_{L} and at least heavy chain constant domains, C_{H}1, C_{H}2, and C_{H}3. The constant domains can be native sequence constant domains (e.g., human native sequence constant domains) or amino acid sequence variant thereof. Preferably, the intact antibody has one or more effector functions.

“Antibody fragments” comprise a portion of an intact antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870, Example 2; Zapata et al., Protein Eng. 8(10): 1057-1062 (1995)); single-chain antibody molecules; and multispecific antibodies formed from antibody fragments. The expression “linear antibodies” generally refers to the antibodies described in Zapata et al., Protein Eng., 8(10):1057-1062 (1995). Briefly, these antibodies comprise a pair of tandem Fd segments (V_{H}-C_{H}1-V_{H}-C_{H}1) which, together with complementary light chain polypeptides, form a pair of antigen binding regions. Linear antibodies can be bispecific or monospecific.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the CHI domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region; this region is also the part recognized by Fc receptors (FcR) found on certain types of cells.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

As used herein, “codon set” refers to a set of different nucleotide triplet sequences used to encode desired variant amino acids. A set of oligonucleotides can be synthesized, for example, by solid phase synthesis, including sequences that represent all possible combinations of nucleotide triplets provided by the codon set and that will encode the desired group of amino acids. A standard form of codon designation is that of the IUB code, which is known in the art and described herein. A codon set typically is represented by 3 capital letters in italics, e.g., NNK, NNS, XYZ, DVK, and the like (e.g., NNK codon refers to N=A/T/G/C at positions 1 and 2 in the codon and K=G/T at equimolar ratio in position 3 to encode all 20 natural amino acids). A “non-random codon set”, as used herein, thus refers to a codon set that encodes select amino acids that fulfill partially, preferably completely, the criteria for amino acid selection as described herein. Synthesis of oligonucleotides with selected nucleotide “degeneracy” at certain positions is well known in that art, for example the TRIM approach (Knappek et al., J. Mol. Biol. 296:57-86, 1999); Garrard and Henner, Gene 128:103, 1993). Such sets of oligonucleotides having certain codon sets can be synthesized using commercial nucleic acid synthesizers (available from, for example, Applied Biosystems, Foster City, CA), or can be obtained commercially (for example, from Life Technologies, Rockville, MD). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain nucleic acid template and also can, but do not necessarily, include restriction enzyme sites useful for, for example, cloning purposes.

An antibody of this invention “which binds” an antigen of interest is one that binds the antigen with sufficient affinity such that the antibody is useful as a diagnostic and/or therapeutic agent in targeting a protein or a cell or tissue expressing the antigen, and does not significantly cross-react with other proteins. In such embodiments, the extent of binding of the antibody to a “non-target” protein will be less than about 10% of the binding of the antibody to its particular target protein as determined by fluorescence activated cell sorting (FACS) analysis or radioimmunoprecipitation (RIA) or ELISA. With regard to the binding of a antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In one embodiment, the term “specific binding” refers to binding where a molecule binds to a particular polypeptide or epitope on a particular polypeptide without substantially binding to any other polypeptide or polypeptide epitope.

“Biologically active” and “biological activity” and “biological characteristics” with respect to a polypeptide of this invention means having the ability to bind to a biological molecule, except where specified otherwise.

“Biological molecule” refers to a nucleic acid, a protein, a carbohydrate, a lipid, and combinations thereof. In one embodiment, the biologic molecule exists in nature.

“Isolated,” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Percent (%) amino acid sequence identity” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc, and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, California. The ALIGN-2 program should be compiled for use on a UNIX operating system, preferably digital UNIX V4.0D. All sequence comparison parameters are set by the ALIGN-2 program and do not vary.

The amino acid sequences described herein are contiguous amino acid sequences unless otherwise specified.

“Structurally unsimilar” biological molecules according to this invention refers to biological molecules that are not in the same class (protein, nucleic acid, lipid, carbohydrates, etc.) or, for example, when referring to proteins, having less than 60% amino acid identity, less than 50% amino acid identity, less than 40% amino acid identity, less than 30% amino acid identity, less than 20% amino acid identity or less than 10% amino acid identity compared to each other.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50 C; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength, and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc, as necessary to accommodate factors such as probe length and the like.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in a animal model such as that disclosed in Clynes et al. (Proc. Natl. Acad. Sci. USA) 95:652-656 (1998).

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells, and neutrophils; with PBMCs and NK cells being preferred. The effector cells can be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202:163 (1996), can be performed.

The term “therapeutically effective amount” refers to an amount of an antibody or antibody fragment to treat a disease or disorder in a subject. In the case of tumor (e.g., a cancerous tumor), the therapeutically effective amount of the antibody or antibody fragment (e.g., a multispecific antibody or antibody fragment for HER2 and VEGF or HER2 and DR5) may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the antibody or antibody fragment may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

By “reduce or inhibit” is meant the ability to cause an overall decrease preferably of 20% or greater, more preferably of 50% or greater, and most preferably of 75%, 85%, 90%, 95%, or greater. Reduce or inhibit can refer to the symptoms of the disorder being treated, the presence or size of metastases, the size of the primary tumor, or the size or number of the blood vessels in angiogenic disorders.

The terms “cancer” and “cancerous” refer to or describe the physiological condition in mammals that is typically characterized by unregulated cell growth. Included in this definition are benign and malignant cancers. By “early stage cancer” is meant a cancer that is not invasive or metastatic or is classified as a Stage 0, I, or II cancer.

The term “precancerous” refers to a condition or a growth that typically precedes or develops into a cancer.

By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer.

An “autoimmune disease” herein is a disease or disorder arising from and directed against an individual's own tissues or a co-segregate or manifestation thereof or resulting condition therefrom. Examples of autoimmune diseases or disorders include, but are not limited to arthritis (rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, and ataxic sclerosis, inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), pyoderma gangrenosum, erythema nodosum, primary sclerosing cholangitis, episcleritis), respiratory distress syndrome, including adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, allergic conditions, allergic reaction, eczema including allergic or atopic eczema, asthma such as asthma bronchiale, bronchial asthma, and autoimmune asthma, conditions involving infiltration of T cells and chronic inflammatory responses, chronic pulmonary inflammatory disease, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis (including large vessel vasculitis (including polymyalgia rheumatica and giant cell (Takayasu's) arteritis), medium vessel vasculitis (including Kawasaki's disease and polyarteritis nodosa), microscopic polyarteritis, CNS vasculitis, necrotizing, cutaneous, or hypersensitivity vasculitis, systemic necrotizing vasculitis, and ANCA-associated vasculitis, such as Churg-Strauss vasculitis or syndrome (CSS)), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, immune complex nephritis, antibody-mediated nephritis, neuromyelitis optica, polyneuropathies, chronic neuropathy such as IgM polyneuropathies or IgM-mediated neuropathy, thrombocytopenia (as developed by myocardial infarction patients, for example), including thrombotic thrombocytopenia purpura (TTP) and autoimmune or immune-mediated thrombocytopenia such as idiopathic thrombocytopeniaurpura (ITP) including chronic or acute ITP, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barré syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis (e.g., benign monoclonal gammopathy and monoclonal garnmopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia areata, CREST syndrome (calcinosis, Raynaud's phenomenon, esophageal dysmotility, sclerodactyly, and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia. Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, infertility due to antispermatozoan antibodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

A “subject” is a vertebrate, preferably a mammal, more preferably a human. Mammals include, but are not limited to, farm animals (such as cows), sport animals, pets (such as cats, dogs and horses), primates, mice, and rats.

Commercially available reagents referred to in the Examples were used according to manufacturer's instructions unless otherwise indicated. The source of those cells identified in the following Examples, and throughout the specification, by ATCC accession numbers is the American Type Culture Collection, Manassas, VA. Unless otherwise noted, the present invention uses standard procedures of recombinant DNA technology, such as those described hereinabove and in the following textbooks: Sambrook et al., supra; Ausubel et al., Current Protocols in Molecular Biology (Green Publishing Associates and Wiley Interscience, N. Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

Throughout this specification and claims, the word “comprise,” or variations such as “comprises” or “comprising,” will be understood to imply the inclusion of a stated integer or group of integers but not the exclusion of any other integer or group of integers.

### II. Therapeutic Uses

The antibodies and antibody fragments described herein which bind both HER2 and VEGF (e.g., bH1 or fragments thereof) or HER2 and DR5 can be used for the treatment of tumors, including pre-cancerous, non-metastatic, and cancerous tumors (e.g., early stage cancer), for the treatment of autoimmune disease, or for the treatment of a subject at risk for developing cancer, for example, breast cancer or an autoimmune disease.

The term cancer embraces a collection of proliferative disorders, including but not limited to pre-cancerous growths, benign tumors, and malignant tumors. Benign tumors remain localized at the site of origin and do not have the capacity to infiltrate, invade, or metastasize to distant sites. Malignant tumors will invade and damage other tissues around them. They can also gain the ability to break off from where they started and spread to other parts of the body (metastasize), usually through the bloodstream or through the lymphatic system where the lymph nodes are located. Primary tumors are classified by the type of tissue from which they arise; metastatic tumors are classified by the tissue type from which the cancer cells are derived. Over time, the cells of a malignant tumor become more abnormal and appear less like normal cells. This change in the appearance of cancer cells is called the tumor grade and cancer cells are described as being well-differentiated, moderately-differentiated, poorly-differentiated, or undifferentiated. Well-differentiated cells are quite normal appearing and resemble the normal cells from which they originated. Undifferentiated cells are cells that have become so abnormal that it is no longer possible to determine the origin of the cells.

The tumor can be a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, polymphocytic leukemia, or hairy cell leukemia), or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further separated into those of epithelial cell origin and those of non-epithelial cell origin. Examples of epithelial cell solid tumors include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organs, bladder, and skin. Solid tumors of non-epithelial origin include sarcomas, brain tumors, and bone tumors.

Epithelial cancers generally evolve from a benign tumor to a preinvasive stage (e.g., carcinoma in situ), to a malignant cancer, which has penetrated the basement membrane and invaded the subepithelial stroma.

Multispecific antibodies that bind both VEGF and HER2 or DR5 and HER2 desirably are used to treat breast cancer.

Examples of autoimmune diseases or disorders include, but are not limited to arthritis (rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, and ataxic sclerosis, inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), pyoderma gangrenosum, erythema nodosum, primary sclerosing cholangitis, episcleritis), respiratory distress syndrome, including adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, allergic conditions, allergic reaction, eczema including allergic or atopic eczema, asthma such as asthma bronchiale, bronchial asthma, and autoimmune asthma, conditions involving infiltration of T cells and chronic inflammatory responses, chronic pulmonary inflammatory disease, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis (including large vessel vasculitis (including polymyalgia rheumatica and giant cell (Takayasu's) arteritis), medium vessel vasculitis (including Kawasaki's disease and polyarteritis nodosa), microscopic polyarteritis, CNS vasculitis, necrotizing, cutaneous, or hypersensitivity vasculitis, systemic necrotizing vasculitis, and ANCA-associated vasculitis, such as Churg-Strauss vasculitis or syndrome (CSS)), temporal arteritis, aplastic anemia, autoimmune aplastic anemia, Coombs positive anemia. Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, immune complex nephritis, antibody-mediated nephritis, neuromyelitis optica, polyneuropathies, chronic neuropathy such as IgM polyneuropathies or IgM-mediated neuropathy, thrombocytopenia (as developed by myocardial infarction patients, for example), including thrombotic thrombocytopenia purpura (TTP) and autoimmune or immune-mediated thrombocytopenia such as idiopathic thrombocytopenia purpura (ITP) including chronic or acute ITP, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barré syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis (e.g., benign monoclonal gammopathy and monoclonal garnmopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia areata, CREST syndrome (calcinosis, Raynaud's phenomenon, esophageal dysmotility, sclerodactyly, and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, infertility due to antispermatozoan antibodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

### III. Dosages and Formulations

The antibody (e.g., bH1) or antibody fragment compositions will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody or antibody fragment to be administered will be governed by such considerations, and is the minimum amount necessary to prevent, ameliorate, or treat a cancer. The antibody or antibody fragment need not be, but is optionally formulated with one or more agents currently used to prevent or treat cancer or a risk of developing a cancer. The effective amount of such other agents depends on the amount of antibody or antibody fragment present in the formulation, the type of disorder or treatment, and other factors discussed above. These are generally used in the same dosages and with administration routes as used hereinbefore or about from 1 to 99% of the heretofore employed dosages. Generally, alleviation or treatment of a cancer involves the lessening of one or more symptoms or medical problems associated with the cancer. The therapeutically effective amount of the drug can accomplish one or a combination of the following: reduce (by at least 10%, 20%, 30%, 40%, 50%, 60%, 70%, 80%, 90%, 100% or more) the number of cancer cells; reduce or inhibit the tumor size or tumor burden; inhibit (i.e., to decrease to some extent and/or stop) cancer cell infiltration into peripheral organs; reduce hormonal secretion in the case of adenomas; reduce vessel density; inhibit tumor metastasis; reduce or inhibit tumor growth; and/or relieve to some extent one or more of the symptoms associated with the cancer. In some embodiments, the antibody or antibody fragment is used to prevent the occurrence or reoccurrence of cancer in the subject.

In one embodiment, the present invention can be used for increasing the duration of survival of a human patient susceptible to or diagnosed with a cancer. Duration of survival is defined as the time from first administration of the drug to death. Duration of survival can also be measured by stratified hazard ratio (HR) of the treatment group versus control group, which represents the risk of death for a patient during the treatment.

In yet another embodiment, the treatment of the present invention significantly increases response rate in a group of human patients susceptible to or diagnosed with a cancer who are treated with various anti-cancer therapies. Response rate is defined as the percentage of treated patients who responded to the treatment. In one aspect, the combination treatment of the invention using an antibody or antibody fragment and surgery, radiation therapy, or one or more chemotherapeutic agents significantly increases response rate in the treated patient group compared to the group treated with surgery, radiation therapy, or chemotherapy alone, the increase having a Chi-square p-value of less than 0.005.

Additional measurements of therapeutic efficacy in the treatment of cancers are described in U.S. Patent Application Publication No. 20050186208.

Therapeutic formulations are prepared using standard methods known in the art by mixing the active ingredient having the desired degree of purity with optional physiologically acceptable carriers, excipients or stabilizers (Remington's Pharmaceutical Sciences (20^{th} edition), ed. A. Gennaro, 2000, Lippincott, Williams & Wilkins, Philadelphia, PA). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, supra.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The antibodies and antibody fragments described herein (e.g., bH1 or fragments thereof) are administered to a human subject, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Local administration may be particularly desired if extensive side effects or toxicity is associated with VEGF, HER2, or DR5 antagonism. An ex vivo strategy can also be used for therapeutic applications. Ex vivo strategies involve transfecting or transducing cells obtained from the subject with a polynucleotide encoding an antibody or antibody fragment. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells.

In one example, the antibody or antibody fragment is administered locally, e.g., by direct injections, when the disorder or location of the tumor permits, and the injections can be repeated periodically. The antibody or antibody fragment can also be delivered systemically to the subject or directly to the tumor cells, e.g., to a tumor or a tumor bed following surgical excision of the tumor, in order to prevent or reduce local recurrence or metastasis.

### Articles of Manufacture and Kits

Another embodiment of the invention is an article of manufacture containing materials useful for the treatment of autoimmune diseases and cancers. Yet another embodiment of the invention is an article of manufacture containing materials useful for the treatment of immunodeficiency diseases. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is a multispecific antibody or antibody fragment antibody of the invention. The label or package insert indicates that the composition is used for treating the particular condition. The label or package insert will further comprise instructions for administering the antibody composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In one embodiment, the package insert indicates that the composition is used for treating breast cancer.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for purification or immunoprecipitation of VEGF, HER2 or DR5 from cells. For isolation and purification of VEGF, HER2, or DR5, the kit can contain a VEGF/HER2 or HER2/DR5 antibody coupled to beads (e.g., sepharose beads). Kits can be provided which contain the antibodies for detection and quantitation of VEGF, HER2, or DR5 in vitro, e.g., in an ELISA or a Western blot. As with the article of manufacture, the kit comprises a container and a label or package insert on or associated with the container. The container holds a composition comprising at least one multispecific antibody or antibody fragment of the invention. Additional containers may be included that contain, e.g., diluents and buffers or control antibodies. The label or package insert may provide a description of the composition as well as instructions for the intended in vitro or diagnostic use.

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1. Library Design and Construction

The antigen-binding site of antibody is formed by the association of the variable domain (V_{H}, V_{L}) of heavy chain (HC) and light chain (LC), each containing three CDR loops for antigen recognition. In many cases one of the two variable domains, often V_{H}, determines the antigen specificity. Mice with transgenic HC but intact LC repertoire generate neutralizing antibody titers (Senn et al., Eur. J. Immunol. 33:950-961, 2003). We set out to investigate how bi-specificity of an antibody can occur and whether different utilization of the V_{H} and the V_{L} domains can enable dual antigen binding specificity.

A semi-empirical approach was taken to find a design for diversifying the amino acid composition and CDR length of antibody light chain and a library template that enabled generation of a functional phage-displayed antibody library from which antibodies binding specifically to a protein antigen could be selected. The sequence and length diversity of the CDR regions of approximately 1500 human kappa light chain sequences, as represented in the Kabat database, served to guide the library design process. Solvent exposed residues were targeted for randomization. A subset of the randomized positions were tailored to represent amino acids which are part of the natural repertoire at these sites, whereas the remaining sites were randomized to include all 20 naturally occurring amino acids.

In particular, the light chain template (variable domain) set forth below was modified as described herein (underlined residues are randomized) (SEQ ID NO:19).

DIQMTQSPSSLSASVGDRVTITCRASQD²⁸VNTAVAWYQQKPGKAPKLLI
YS⁵⁰ASFLYSGVPSRFSGSGSGTDFTLTISSLQPEDFATYYCQQH⁹¹YTT
PPTFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC

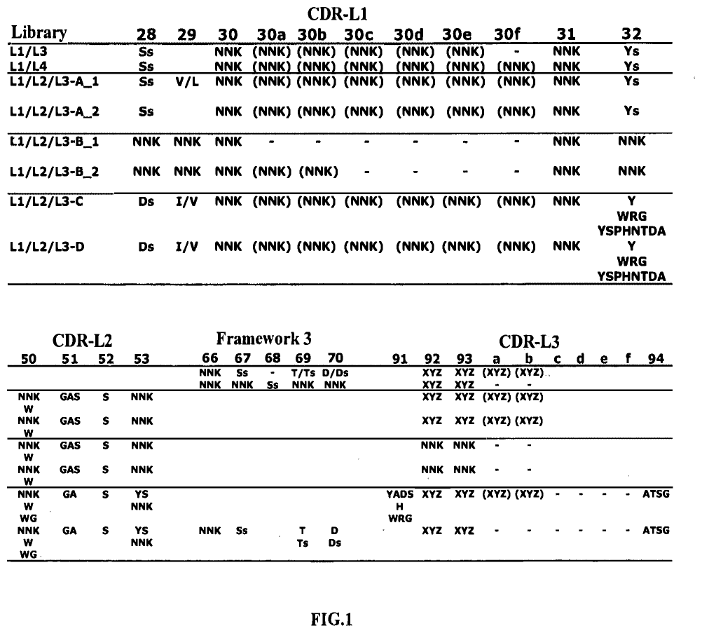

Four sets of libraries were generated based on 3 human Fab and scFv templates where distinct sets of positions were targeted for randomization (FIG. 1).

In all of the libraries the heavy chain was held constant with its sequence defined by the library template. The heavy chain template (variable domain) sequence is set forth below (SEQ ID NO:20).

EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTH

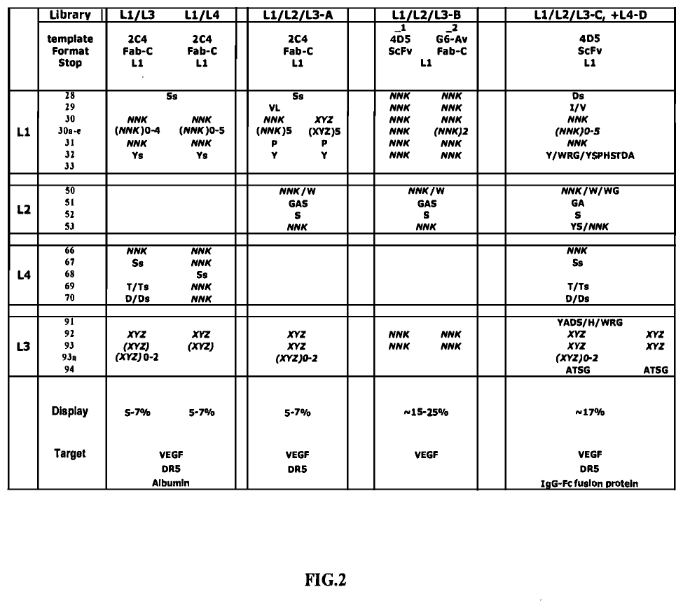

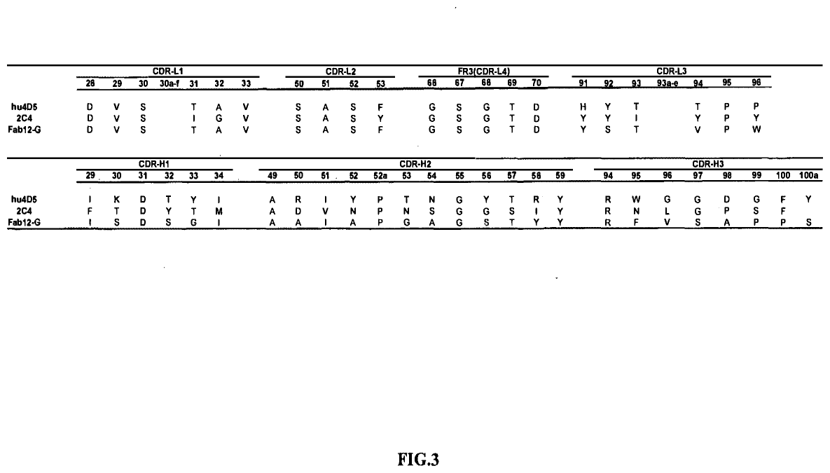

All library templates contained a stop codon (Sidhu et al., 2004) embedded in CDR L1 preventing the presence of template light chain among the phage-displayed antibody library members. The template CDR sequences are summarized in FIG. 3. The library designs are summarized in FIG. 1 and FIG. 2.

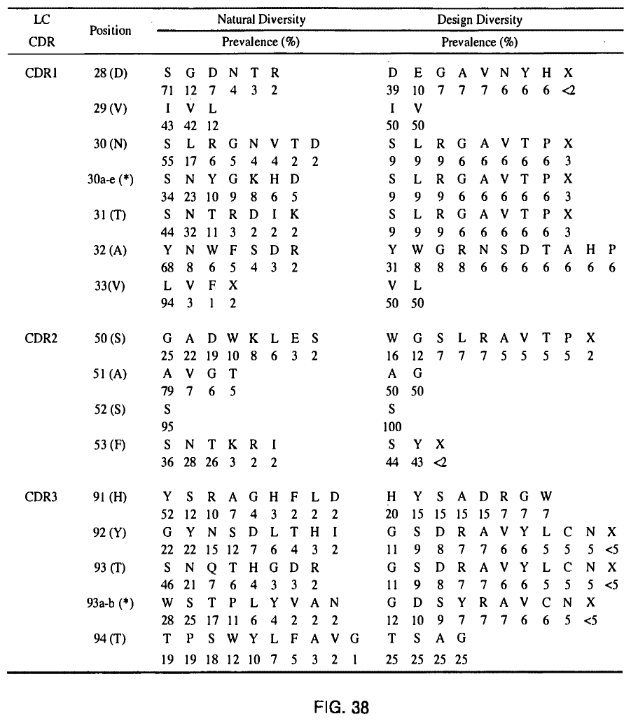

In one example, we introduced mutations in the LC variable domain of a HER2-specific antibody to identify variants that can bind a different protein antigen while retaining the original binding specificity. We took a conservative approach to randomize the LC CDRs in order to generate variants that can be stably expressed. Twelve solvent exposed LC CDR positions were selected for randomization: five in CDR1 (28, 29, 30, 31, 32), three in CDR2 (50, 51, 53) and four in CDR3 (91, 92, 93, 94). Further, to guide the design of amino acid diversity at elected sites, the natural diversity of these positions was examined by analysis of approximately 1500 human kappa LC CDR sequences (Johnson and Wu, Nucleic Acids Res. 28:214, 2000; Chothia and Lesk, J. Mol. Biol. 196:901, 1987) (FIG. 38). Some positions with relatively high natural diversity (30, 31, 50, 92, 93) were fully randomized while other positions were limited to as few as two amino acid types to mimic natural antibodies. The length variation of natural LC CDR1 and CDR3 was also reflected in the library (FIG. 38). In FIG. 38, X denotes the amino acid types designed at low frequencies as shown. Length diversity is constructed by inserting 1 to 5 residues between residues 30 and 31 and between residues 93 and 94.

The LC library is a productive naive repertoire (Table 1). Listed are results from the screening of 95 random clones at the end of four rounds of selection. In particular, selection for new binding specificity was performed as described on immobilized targets (VEGF, DR5, and human Fc) (Sidhu et al., J. Mol. Biol. 338:299, 2004). After four rounds of selection 95 phage clones were assayed using ELISA for binding to the target, HER2, and a non-target protein, BSA, to ensure specific binding. To enrich for target binding clones that maintained HER2 binding, a final round of selection on HER2 was performed. The positive clones were sequenced. To identify the highest affinity binders, the IC₅₀ for antigen binding was determined by competitive ELISA (Sidhu et al., J. Mol. Biol. 338:299, 2004). The number of unique clones as determined by sequence analysis and the number of unique clones that maintain HER2 binding (bispecific clones) are shown. These clones show minimum background binding signals to irrelevant antigens, such as BSA.

TABLE 1
Light chain library selection summary

| | Positive % | Unique Seq. | HER2 positive |
|---|---|---|---|
| Human Fc fusion | 63 | 31 out of 61 | 1 |
| hVEGF | 77 | 41 out of 74 | 30 out of 41 |
| DR5 long | 85 | 5 out of 82 | 2* out of 5 |

*= weak binding signal

| Target | Bi-Specific, Screen | Bi-Specific, Selection |
|---|---|---|
| Human Fc fusion | 1 out of 31 | Not determined |
| hVEGF | 30 out of 41 | 94 out of 94 |
| DR5 long | 2* out of 5 | 2 out of 7** |

*= weak binding signal Her2
**= weak binding signal DR5

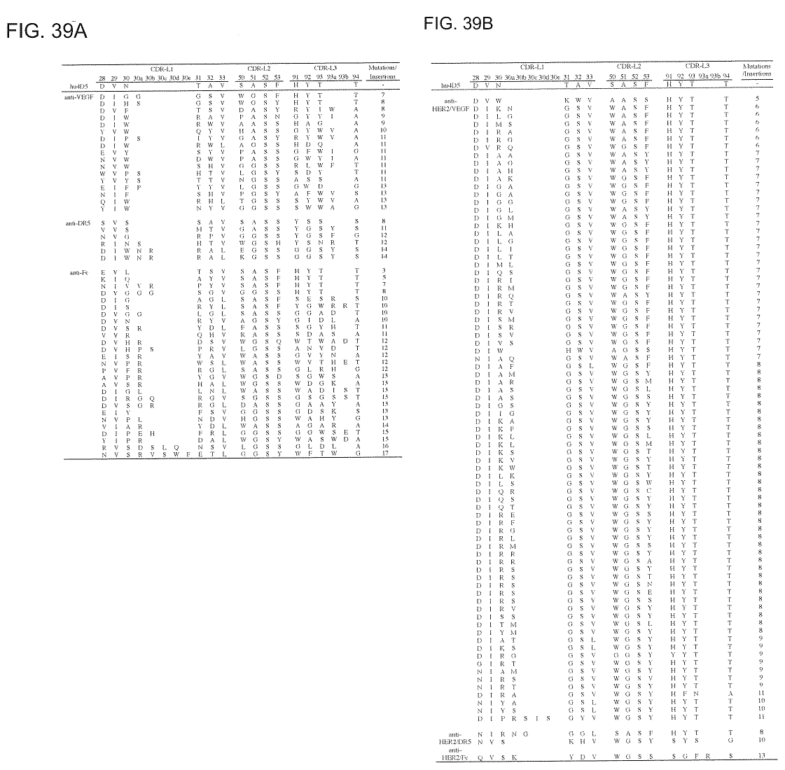

Selection against three protein antigens: human vascular endothelial growth factor (hVEGF), death receptor 5 (DR5), and complement binding fragment of IgG (Fc) generated many binding clones (FIG. 39A). Some clones lost binding affinity for HER2, while others maintained HER2-binding and were thus bi-specific. Sequence analysis of the 131 unique Herceptin® antibody variants with new binding specificity identified the amino acid substitutions and insertions compared to the Herceptin® antibody (FIG. 39B).

The number of mutations ranged from 3-17. The clones that retained HER2 binding (the bi-specific clones) contained fewer mutations on average than those that lost HER2 binding. Retaining the Herceptin® antibody CDR-L3 sequence was preferred but not sufficient to conserve HER2 binding. This is consistent with the report that the Herceptin® antibody CDR-L3 is the most important LC CDR for HER2 binding (Kelley and O'Connell, Biochemistry 32:6828, 1993). Representative VEGF and DR5-binding clones were expressed as Fab and IgG proteins (Table 2).

TABLE 2
The representative antibodies isolated from the light chain library
of the Herceptin ® antibody (SEQ ID NOS: 21-42).

| | CDR-L1 | | | | | | | | | | CDR-L2 | | | | CDR-L3 | | | | | | | |
|---|---|---|---|---|---|---|---|---|---|---|---|---|---|---|---|---|---|---|---|---|---|---|
| Her- | 28 | 29 | 30 | 30a | 30b | 30c | 30d | 31 | 32 | 33 | 50 | 51 | 52 | 53 | 91 | 92 | 93 | 93a | 93b | 94 | Specificity | K_{D}(nM) |
| ceptin | D | V | N | — | — | — | — | T | A | V | S | A | S | F | H | Y | T | | | T | HER2 | 0.1 |
| 3-1 | N^{a} | V | W | | | | | D | W | V | P | A | S | S | G | W | Y | I | | A | VEGF | 15 |
| bH1 | D | I | P | R | S | I | S | G | Y | V | W | G | S | Y | H | Y | T | | | T | | 300/26 |
| bH3 | D | I | G | L | | | | G | S | V | W | A | S | Y | H | Y | T | | | T | VEGF/HER2 | 19,000/8 |
| bH4 | D | I | R | S | | | | G | S | V | W | G | S | Y | H | Y | T | | | T | | 3,500/11 |
| 4-1 | D | I | W | N | R | | | R | A | L | E | G | S | S | G | G | S | Y | S | S | DR5 | 120 |
| 4-5 | N | V | G | | | | | R | P | V | G | G | S | S | Y | G | S | F | G | T | | 150 |
| bD1 | N | V | S | | | | | K | H | V | W | G | S | Y | S | Y | S | | | G | DR5/HER2 | 14,000/200 |
| bD2 | N | I | R | N | G | | | G | G | L | S | A | S | F | H | Y | T | | | T | | 67,000/100 |

^{a}Differences from Herceptin are shown in bold

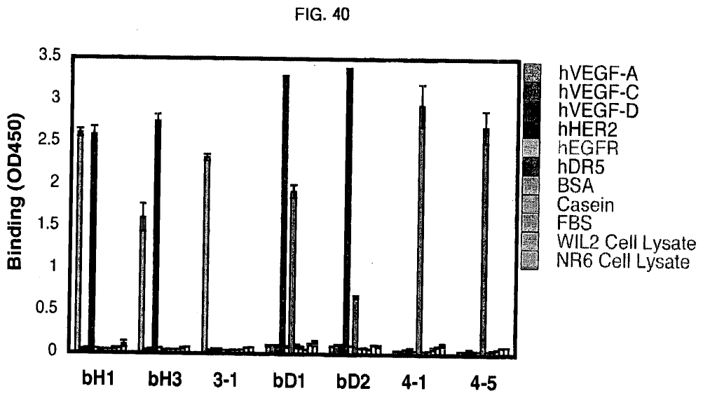

To demonstrate that these antibodies bound specifically to their cognate antigens and did not interact non-specifically with other proteins, we showed that there was no detectable binding to a panel of mammalian cell lysates and non-antigen proteins. The assay confirmed the mono- and bi-specificity of the purified IgGs or Fabs (FIG. 40).

Equilibrium binding affinities (K_{D}) of the LC library-derived mono-specific antibodies ranged from 15-150 nM. The bi-specific antibodies bound the new antigens (i.e., VEGF and DR5) with high nM to low μM affinity and HER2 with low nM affinity (Table 2). The antibody bH1 displayed the highest bi-specific affinity for the two different protein antigens VEGF (K_{D}=300 nM) and HER2 (K_{D}=26 nM).

### Materials

Enzymes and M13-KO7 helper phage were from New England Biolabs. E. coli XL1-Blue was from Stratagene. Bovine serum albumin (BSA), ovalbumin, and Tween 20 were from Sigma. Neutravidin, casein, and Superblock were from Pierce. Immobilized protein G and anti-M13 conjugated horse-radish peroxidase (HRP) were from GE Healthcare (Piscataway, NJ). Maxisorp immunoplates were from NUNC (Roskilde, Denmark). Tetramethylbenzidine (TMB) substrate was from Kirkegaard and Perry Laboratories (Gaithersburg, MD). All protein antigens were generated by research groups at Genentech, Inc. DNA degeneracies were represented using the IUB code and represent equimolar mixtures unless indicated otherwise: N=A/C/G/T, D=A/G/T, V=A/C/G, B=C/G/T, H=A/C/T, K=G/T, M=A/C, R=A/G, S=G/C, W=A/T, Y=C/T.

For example, at certain randomized positions, the wild-type codon was replaced by a degenerate NNK codon (N=A/T/G/C, K=G/T in an equimolar ratio) that encodes all 20 natural amino acids. The XYZ codon refers to a codon with unequal nucleotide ratios at each position of the codon triplet. X contained 38% G, 19% A, 26% T and 17% C; Y contained 31% G, 34% A, 17% T and 18% C; and Z contained 24% G and 76% C.

### Phagemid Vectors for Library Construction

Standard molecular biology techniques were used for vector construction. Three templates were constructed for library generation. All templates are derivatives of plasmid pV0354 used in heavy chain libraries based on modified humanized 4D5 (version 8) (Lee et al., 2004a).

The 2C4 Fab-C template phagemid pJB0290 was constructed by cloning the 2C4 heavy chain variable domain into a pV0354-Fab-C vector containing the alkaline phosphatase promoter (Lowman et al., 1991) and stII secretion signal for both light and heavy chain of Fab. It is engineered to contain a single cysteine at the C-terminus of the heavy chain variable domain 1 to allow bivalent M13 phage display of the 2C4 Fab as previously described (Lee et al., 2004b). The 2C4 light chain CDRs were incorporated into the Fab-C vector by site-directed mutagenesis using the method of Kunkel et al (Kunkel et al., 1987). An epitope tag (gD tag) (Lasky and Dowbenko, 1984) was added at the C-terminus of the light chain to enable determination of the level of display as described (Sidhu et al., 2004). The Fab12-G library template pV1283 was created by cloning a highly displayed heavy chain variable domain into pV0354-Fab-C, and the light chain variable domain was modified to contain CDR-L3 of Fab-12 (humanized A4.6.1, an anti-VEGF antibody). The highly-displayed V_{H} was selected from a Fab library that randomized heavy chain CDR residues of G6 Fab using shotgun alanine scanning mutagenesis (Liang et al., 2006; Vajdos et al., 2002) with CDR-L3 converted to Fab-12 (Y₉₁STVPW₉₆) by panning on immobilized anti-gD antibody. The design and construction of the phagemid pV1384, displaying 4d5 (LC-R66G) scFv bivalently on the surface of M13 phage particles was modified from the template pS2018 described previously (Sidhu et al., 2004). The scFv fragment contained a gD epitope tag in the linker region between light chain and heavy chain. LC framework residue Arg66 was mutated to Gly66, which is the prevalent residue in this position in over 95% of natural kappa light chains. The mutation R66G reduces Herceptin® antibody binding affinity to HER2 only slightly (<2 fold) as described in Kelley and Connell (Biochemistry 32:6828, 1993). The CDR sequences of the library templates are summarized in FIG. 3.

### Library Construction

Phage-displayed libraries were created using oligonucleotide-directed mutagenesis as described (Sidhu et al., 2004). The library template vectors contained a stop codon (TAA) embedded in CDR-L1, which was repaired during the mutagenesis reaction using degenerate oligonucleotides that annealed over the sequences encoding CDR-L1, CDR-L3 (all libraries), CDR-L2 (L1/L2/L3-A, -B, -C, +L4-D) and the light chain framework 3 (L1/L4 and L1/L2/L3+L4-D). The library mutagenesis reactions were performed according to the method of Kunkel et al (Kunkel et al., 1987). The light chain CDR designs for the libraries are described in FIG. 1, which summarizes the degenerate codons used at each position for the different libraries. Three or four oligonucleotides were mixed at certain ratios for each CDR to encode the desired frequency of amino acid types at each position targeted for randomization (FIG. 38). The oligonucleotides were combined in different ratios to fine-tune the diversity to reflect the amino acid frequency in natural light chain kappa sequences at selected positions. For CDR1, three oligonucleotides containing codons for positions 91-94: CAT NNK NNK RST (SEQ ID NO:43), KMT XYZ XYZ RST (SEQ ID NO:44), or DGG XYZ XYZ RST (SEQ ID NO:45) were mixed at 1:3:1 ratios. XYZ is a variation of NNK that has equal proportions of the A/G/T/C for each site to reduce the coverage of aliphatic hydrophobic amino acids (Lee et al., J. Mol. Biol. 340:1073, 2004). For CDR2, four oligonucleotides containing codons for positions 50-53: NNK GST TCC NNK (SEQ ID NO:46), TGG GST TCC NNK (SEQ ID NO:47), KGG GST TCC TMT (SEQ ID NO:48), or NNK GST TCC TMT (SEQ ID NO:49) were mixed at 1:1:2:10 ratios. For CDR3, each length was a mixture of three oligonucleotides containing codons for position 28-33: G₇₀A₇₀C₇₀ RTT NNK NNK TAC STA (SEQ ID NO:50), G₇₀A₇₀C₇₀ RTT NNK NNK DGG STA (SEQ ID NO:51), or G₇₀A₇₀C₇₀ RTT NNK NNK NMT STA (SEQ ID NO:52) at 1:1:2 ratios. G₇₀A₇₀C₇₀ is a “soft” codon that allows 70% of the designated nucleotide and 10% each of the other three, encoding ˜50% of Glu and ˜50% of the other amino acids.

Structural analysis of a number of representative antibodies with kappa LCs shows that CDR1 has the widest range of conformations, which is likely a result of the variation in loop lengths (11-17 residues between position 24 and 34). Different CDR-L1 lengths (lengths 11-16) were thus included in the library. Natural CDR-L3 also varies in length (lengths 7-10 residues between position 89-96), which is reflected by the library design (lengths 8-10; FIG. 38).

FIG. 1 shows the comparison of the light chain natural diversity and the actual library designs. The mutagenesis products were pooled into one reaction per library and electroporated into E. coli SS320 cells supplemented with KO7 helper phage and were grown overnight at 30° C. (Lee et al., J. Mol. Biol. 340:1073, 2004). ˜10¹¹ cells and ˜5-10 μg DNA were used in each electroporation reaction. The library phage were purified (Sidhu et al., J. Mol. Biol. 338:299, 2004). The number of transformants ranged from 10⁹-10¹⁰. The display level of intact Fabs or scFv on the surface of phage was determined in an ELISA binding assay where 96 randomly selected clones from each library were tested for their ability to bind an anti-gD antibody. The display level ranged from 5-25% (FIG. 2). 25% of the clones displaying antibody retained HER2 binding. Approximately 150 displaying clones were sequenced to examine the actual library diversity as compared to the design diversity. A portion (˜30%) of the functionally displayed library members retained the Herceptin® antibody CDR-L2 and/or CDR-L3 sequence due to incomplete mutagenesis (a template stop codon in CDR-1 ensured 100% mutation of this CDR in expressed scFvs). These were excluded from the sequence analysis of the actual library diversity. At the majority of the randomized positions, the diversity of the phage displayed library of the displaying clones did not deviate significantly (p>0.05, odds ratio test) from the designed diversity. Exceptions were position 29 of the CDR-L1 where Val was found to be slightly over-represented compared to Ile (p=0.005) and positions 51 and 53 of CDR-L2, where Gly and Ser were more prevalent than Ala and Tyr, respectively (p<0.01).

### Example 2. Evaluation of Library Performance

### Library Sorting and Screening

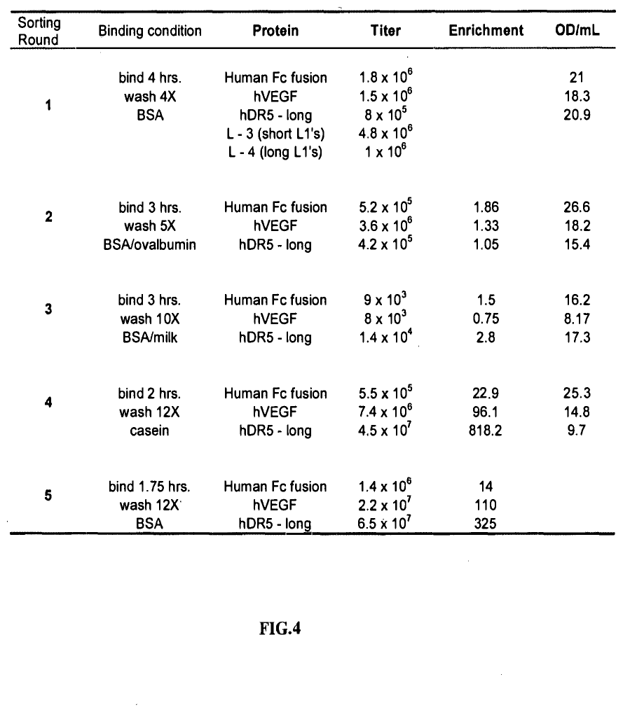

A library was considered functional when antibodies binding specifically to various protein antigens could be isolated after 4-5 rounds of sorting. Many protein targets were known to allow functional immobilization for library panning and specific antibodies have been generated from validated phage-displayed libraries (Fellouse et al., 2005) (Lee et al., 2004a). To evaluate each set of libraries, we chose a subset of these targets for selection (FIG. 2). The libraries were subjected to an initial round of binding selection with anti-gD antibody or protein L as the capture target to eliminate clones in which the Fab/scFv gene had been deleted, followed by 4-5 rounds of antigen selection. Alternatively, they were directly subjected to target binding selection without pre-selection with anti-gD or protein L. NUNC 96-well Maxisorp plates were coated overnight with antigen (5 μg/ml) and blocked for 1 hour with alternating blocking agents (FIG. 4). Phage solutions of 10¹³ phage/ml were added to the coated immunoplates in the first selection cycle. The phage concentration was decreased in each round of selection. Following incubation of the phage solutions on the immunoplates to allow binding to the immobilized antigen, the plates were washed with PBS, 0.5% Tween 20, repeatedly. To increase the stringency, the incubation time was decreased (4 hours for 1^{st} round, 3 hours 2^{nd}, 3 hours 3^{d}, 2 hours 4^{th}, 1.75 hours 5^{th}) and the number of washes was increased in each round of selection (FIG. 4). Bound phage was eluted with 0.1 M HCl for 30 minutes and the eluant was neutralized with 1.0 M Tris base. The recovery of phage per antigen-coated immunoplate well was calculated and compared to that of a blocked well without coated antigen to study the enrichment of phage clones displaying Fabs or scFvs that specifically bound the target antigen (FIG. 4). Eluted phage were amplified in E. coli and used for further rounds of selection. Random clones from rounds 4 and 5 were selected for screening and assayed using phage ELISA in which binding to target and anti-gD was compared to binding of a non-relevant protein (BSA) for checking non-specific binding. Clones that bound the anti-gD antibody and target but not the non-specific protein were considered specific positives. Libraries L1/L3, L1/L4, L1/L2/L3-A, L1/L2/L3-B_1 and L1/L2/L3-B_2 did not yield any specific positive clones whereas libraries L1/L2/L3-C and L1/L2/L3+L4-D enabled isolation of specific antibodies to the target antigens.

For example, random clones from round four were assayed using phage ELISA where binding of individually amplified clones to the target and HER2 was compared to binding of a non-target protein (BSA) to test binding specificity. To enrich the phage clones that maintained HER2 binding, the eluted phage from the third and fourth round of VEGF or DR5 selection were amplified and subjected to another round of selection on HER2 coated wells. The V_{L} and V_{H} regions of the positive clones were amplified by PCR and sequenced.

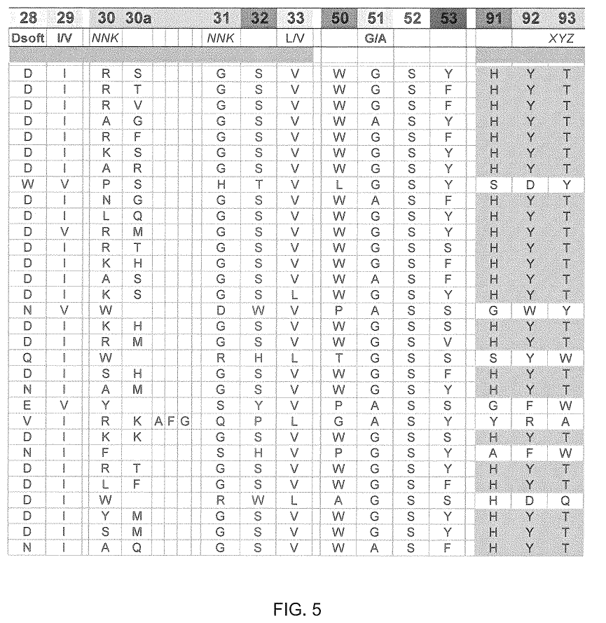

The hit rate for hFC, hVEGF and hDR5-If, was 63, 77, and 85% respectively. The V_{L} regions of the positive clones were amplified by PCR and sequenced as described (Sidhu et al., 2004). The DNA sequence analysis of the positive specific binders revealed a percentage of unique clones of 51% (hFC), 55% (hVEGF), and 6.1% (hDR5-If). The sequences of unique hVEGF, hDR5 and hFc binding clones are summarized in FIG. 5 and FIG. 6, respectively.

Combined Plate and Solution Selection of hVEGF Binding Clones

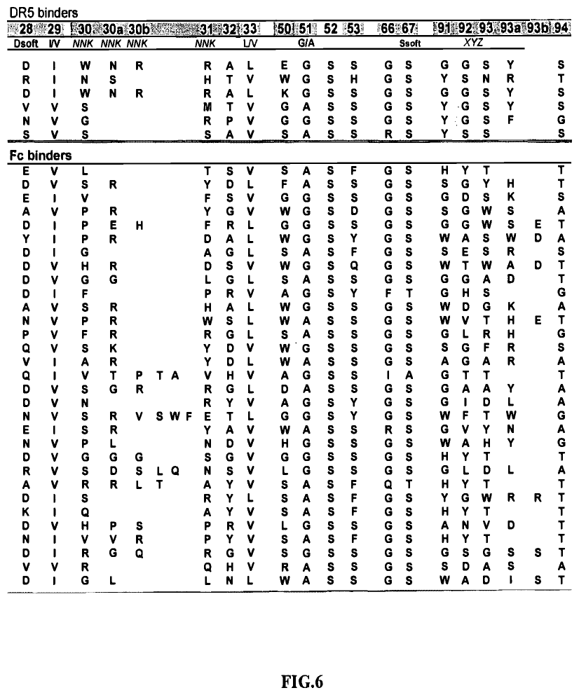

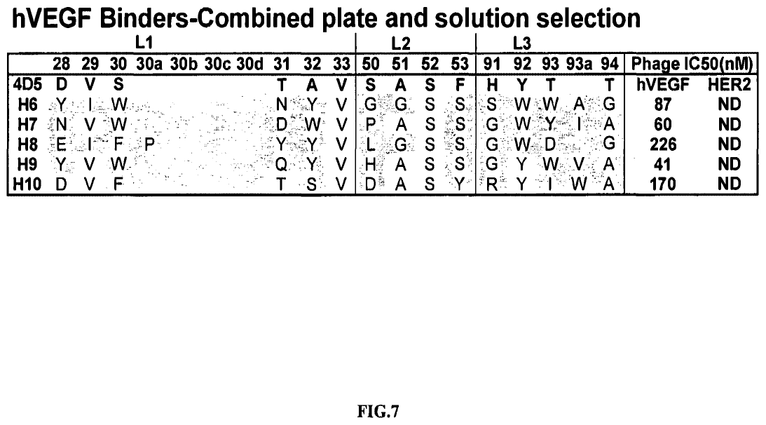

High diversity of hVEGF binding clones after four rounds of sorting was observed. In order to identify high affinity hVEGF binding clones a solution based selection approach was taken following the 4^{th} plate based sort. 50 nM biotinylated hVEGF was incubated with the phage propagated from the 4^{th} round of selection on immobilized antigen. After 2 hours of incubation at room temperature with shaking. hVEGF-bound phage was captured on neutravidin-coated and blocked immunoplates followed by repeated washes. Phage clones were eluted, screened, and sequenced as previously described. Sequences of hVEGF binding clones from the last solution selection step are found in FIG. 7.

Isolation of Bi-Specific Clones from Libraries L1/L2/L3-C and L1/L2/L3+L4-D

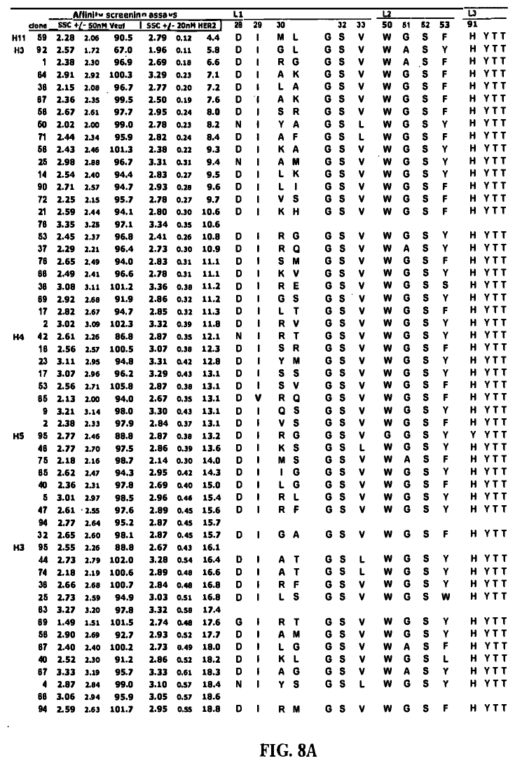

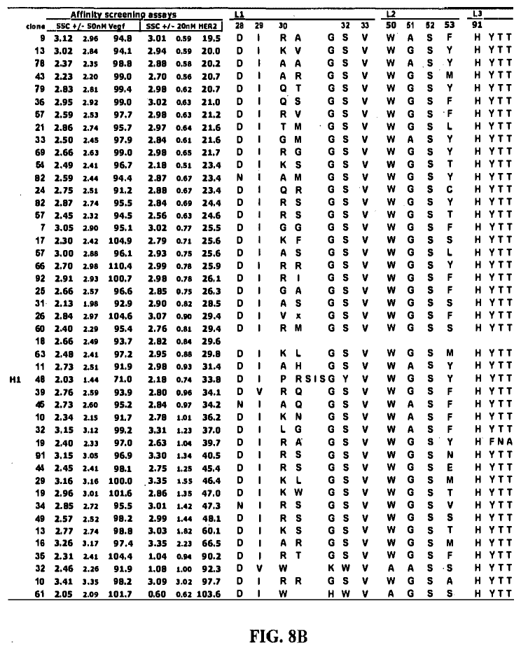

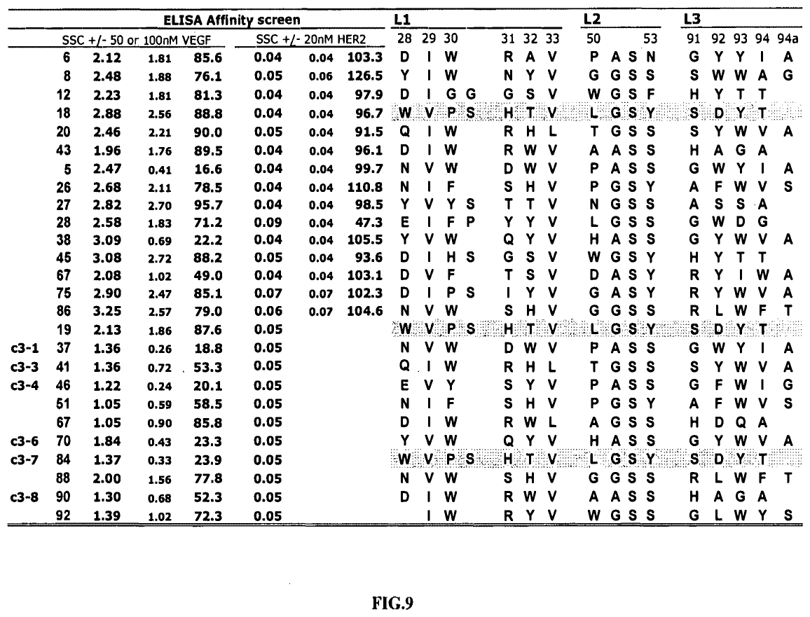

The library template for libraries L1/L2/L3-C and L1/L2/L3+L4-D was an scFv fragment modified from the hu4D5 antibody, which binds Her2 with high affinity. Mapping of the functional paratope of hu4D5-5 for Her2 binding by alanine-scan mutagenesis of the CDR regions showed that heavy chain residues contribute the majority of the free energy of binding, whereas individual light chain residues contribute to a lesser extent (Kelley and O'Connell, 1993). Analysis of the atomic structure of the Herceptin® antibody Fab in complex with human Her2-ECD demonstrates that while the light chain is involved in making antigen contact, the heavy chain provides most of the structural interface with the antigen (Cho et al., Nature 421:756, 2003). We observed that some members of the functional light chain libraries built upon Herceptin® antibody template retained Her2 binding ability. In an attempt to isolate bi-specific scFv fragments from the functional libraries L1/L2/L3-C and L1/L2/L3+L4-D, capable of binding Her2 as well as a second antigen, two strategies were applied. In one approach the positive clones from the previously described target antigen selection was screened by ELISA for ones that retained Her2 binding. The percentage of specific positive clones capable of binding Her2 varied depending on the second antigen specificity. Only 1 out of 61 unique hFc specific positive clones clone still bound Her2 (1.6%), 30 out of 41 unique hVEGF binding clones still bound Her2 (73%), and 2 out of 5 unique hDR5 binders still bound Her2 (40%). In addition, a selection-based approach was taken to isolate bi-specific antibodies by selecting Her2 binders from the pool of hVEGF and hDR5 binding antibodies. The elution from round 4 of target antigen sorting was subjected to an additional round of selection by incubating 2×10¹³ phage/ml on Her2 coated (5 μg/ml) and BSA-blocked Maxisorp immunoplates for 1 hour. The plates were washed 15 times with PBS, 0.5% Tween 20 and bound phage eluted as described previously. Random clones were selected and assayed for Her2, anti-gD and target binding and compared to non-specific binding to an un-relevant protein (BSA). All 192 clones tested were identified as specific positives and sequenced as described previously. Sequencing revealed 94 unique sequences. In summary, this method generated 94 Her2/hVEGF bi-specific clones out of the 94 unique clones tested (100%) (FIG. 5). The sequences of all isolated unique hVEGF/Her2 bi-specific antibodies from both isolation strategies are summarized in FIG. 8. The sequences of isolated clones that lost all detectable binding to Her2 are shown in FIG. 9. Of the clones that have dual specificity, nearly all retained the Herceptin® antibody CDR-L3, making it likely that maintaining CDR-L3 is important for maintaining HER2 binding. In the case of hDR5, 2 out of the 7 unique Her2-binding clones were bi-specific (29%, 12 clones sequenced). The sequences of these clones are summarized in FIG. 10. One of the dual specific clones had some homologous changes in CDR-L3.

High-Throughput Characterization of hVEGF Binding Clones

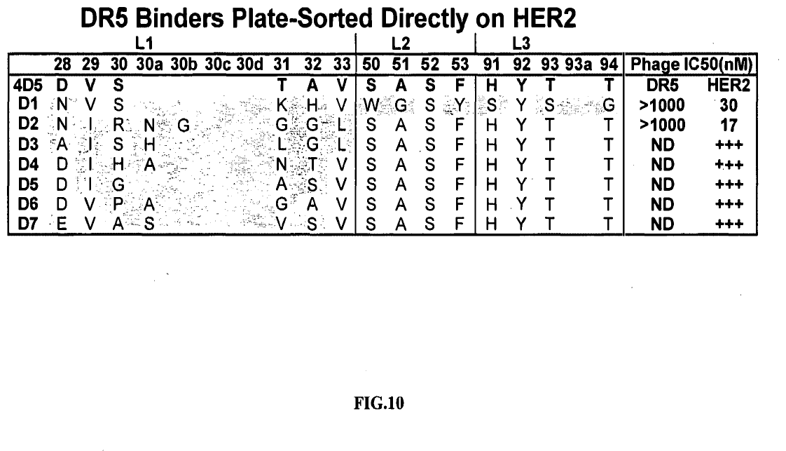

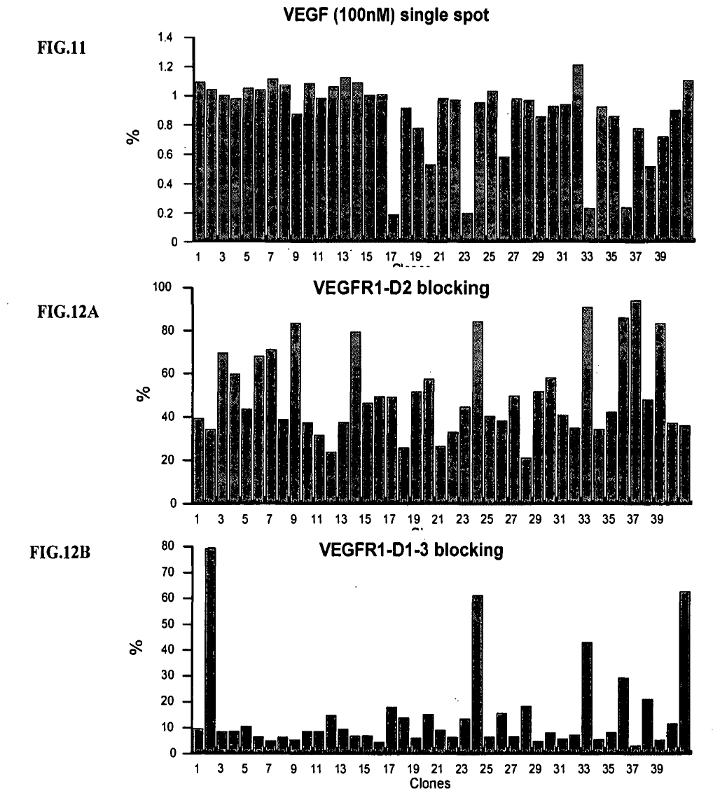

A high-throughput single spot competitive ELISA in a 96-well format (Sidhu et al., 2004) was used to screen for high affinity clones for hVEGF and to study the VEGFR1-blocking profiles. Briefly, Maxisorp Immunoplates were coated with 2 μg/ml hVEGF₁₀₉, overnight at 4° C., and blocked with 1% (w/v) BSA for 1 hour. Phagemid clones in E. coli XL1-Blue were grown in 150 μl of 2YT broth supplemented with carbenicillin and M13-KO7 helper phage; the cultures were grown with shaking overnight at 37° C. in a 96-well format. Culture supernatants containing phage were diluted fivefold in PBST (PBS with 0.05% Tween 20 and 0.5% (w/v) BSA) with or without the addition of 100 nM hVEGF₁₀₉ for affinity screen. For receptor blocking screens, hVEGF coated wells were incubated with or without VEGFR1 Domain 1-3 (D1-3) and VEGFR1 Domain 2 (D2) before adding five-fold diluted phage supernatant (Liang et al., 2006; Wiesmann et al., 1997). After incubation for 1 hour at room temperature (RT), the mixtures were transferred to the coated plates with hVEGF₁₀₉ and incubated for 10 minutes. The plate was washed with PBT (PBS with 0.05% Tween 20) and incubated for 30 minutes with anti-M13 antibody horse-radish peroxidase conjugate diluted 5000-fold to 1 nM in PBST. The plates were washed, developed with TMB substrate for approximately five minutes, quenched with 1.0 M H₃PO₄, and read spectrophotometrically at 450 nm. In the single-spot affinity assay, the ratio of the absorbance in the presence of solution-phase h VEGF₁₀₉ to that in the absence of solution-phase hVEGF₁₀₉ was used as an indication of the affinity. A low ratio would indicate that most of the Fab-phage were bound to solution-phase hVEGF₁₀₉ in the initial incubation stage and, therefore, were unavailable for capture by immobilized hVEGF₁₀₉. The high-throughput affinity assay results of the first 41 unique clones are summarized in FIG. 11. Similarly, for the blocking assay, a low ratio indicated that the binding of a clone to hVEGF₁₀₉ is blocked by the hVEGF₁₀₉-VEGFR1 interaction, indicating that some clones have an overlapping binding site (epitope) on VEGF with the respective VEGF receptor fragments (FIG. 12) and these clones are likely to be displaying the blocking antibodies.

High-Throughput Characterization of Bi-Specific hVEGF/Her2 Clones

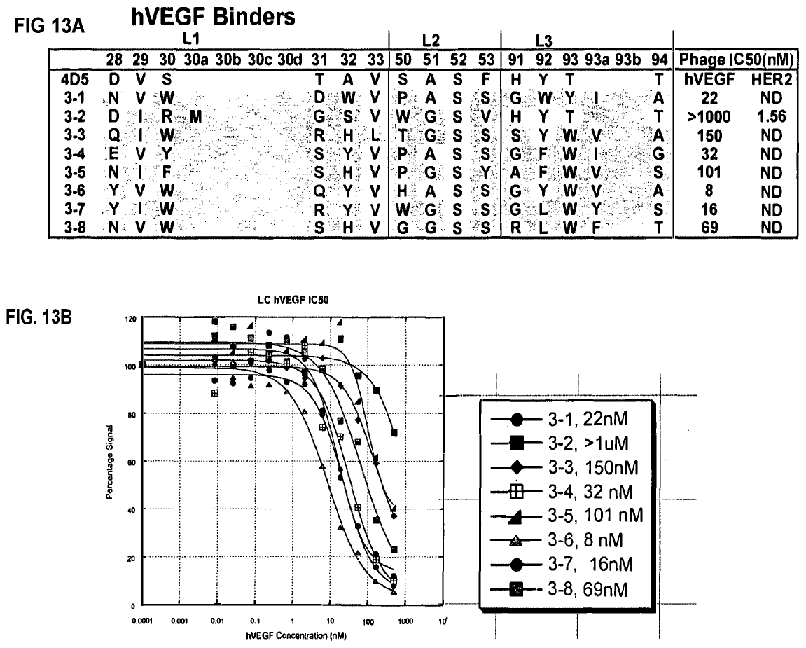

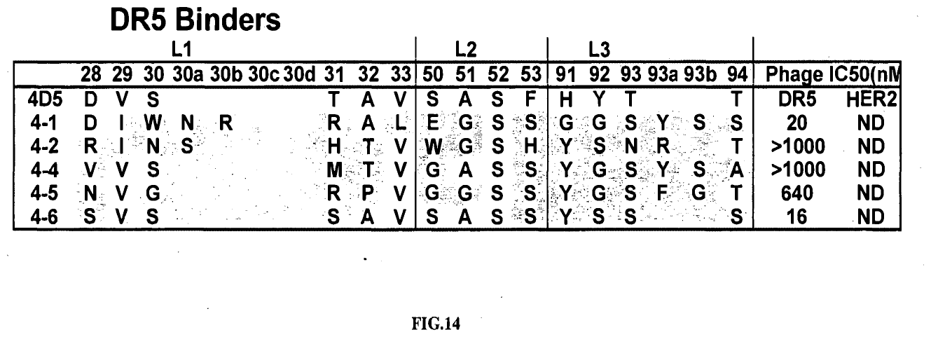

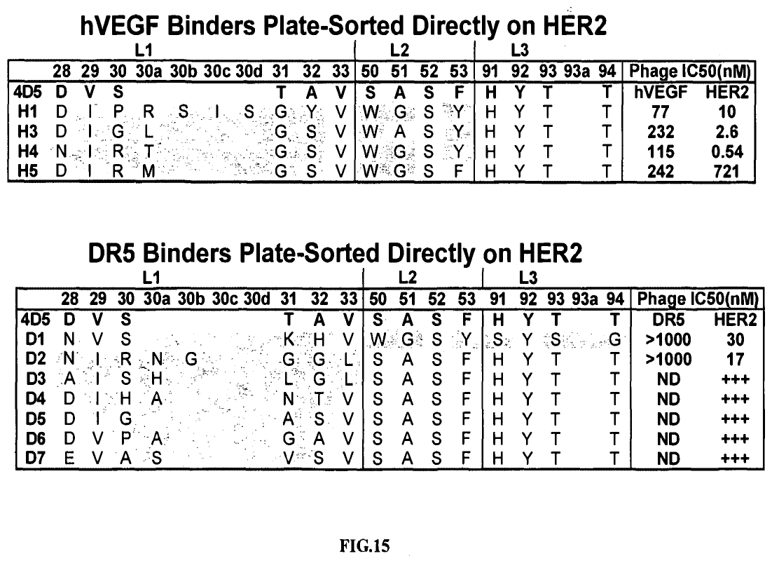

The same principle as described in the previous section was applied to enable isolation of clones with high affinity for hVEGF and Her2 for further characterization. The high-throughput single point competitive ELISA was used to screen for high affinity clones for hVEGF and Her2 by coating Maxisorp Immunoplates with 2 μg/ml hVEGF₁₀₉, and Her2-ECD overnight at 4° C., followed by blocking with 1% (w/v) BSA for 1 hour. Phage clones that were identified as bi-specific in the previous single spot ELISA screen were grown as described previously and incubated with and without the addition of 20 nM Her2-ECD and 50 nM hVEGF. After incubation for 1 hour at room temperature, the solutions were applied to the coated immunoplates and the binding signals recorded and analyzed as described in the previous section. Clones with low ratio for both hVEGF and Her2 were selected for further characterization. hVEGF-specific and hVEGF/Her2 bi-specific phage clones that gave rise to the lowest signal ratios in the single spot competitive ELISA were selected for affinity measurement by competitive ELISA as well as the DR5-binding and DR5/Her2 bi-specific phage clones from the initial single spot ELISA screen and VEGF binding clones from the combined plate and solution selection. Phage clones were propagated from a single colony by growing in 25 ml of 2YT culture supplemented with carbenicillin and KO7 helper phage overnight at 30° C. Phage purified by precipitation in PEG/NaCl were first diluted serially in PBST and tested for binding to an antigen-coated plate. The dilution that gave 50-70% saturating signal was used in the solution binding assay in which phage were first incubated with increasing concentration of antigen for one to two hours and then transferred to antigen-coated plates for 10-15 minutes to capture the unbound phage. IC₅₀ was calculated as the concentration of antigen in solution-binding stage that inhibited 50% of the phage from binding to immobilized antigen (Lee et al., 2004a). FIG. 13B depicts the curves from which the IC₅₀ was calculated for the analyzed hVEGF binding clones from the plate sorting strategy. The IC₅₀ values ranged from 22 nM to >1 μM (FIG. 13B). The IC₅₀ values for the hVEGF binders isolated by combined plate and solution based selection ranged from 41 nM-226 nM (FIG. 7). IC₅₀ values of DR5-binding clones ranged from 20 nM to >1 μM (FIG. 14). The IC₅₀ values for hVEGF/Her2 and DR5/Her2 bi-specific clones are summarized in FIG. 15A and FIG. 15B. IC₅₀ values for Fc binding clones have not been determined.

### Example 3. Characterization of Antibodies from the Light Chain Library

Conversion of scFvs to Fabs

To test whether conversion of the scFvs′2 as displayed on phage to Fabs affected the affinity of the binding clones from the library, 2 clones (3-7 anti-hVEGF and 4-1 anti-hDR5) were chosen for conversion to Fab and displayed on phage. The V_{L} region of Phagemid DNA for selected hVEGF and DR5 scFv fragments was digested with restriction enzymes, which cleaved the DNA upstream of the region encoding for CDR-L1 (EcoRV) and downstream of the region encoding for CDR-L3 (KpnI). The digested DNA fragment was ligated into a similarly digested vector (pAP2009) designed for the phage display of Fab hu4D5 by fusion to the C-terminal domain of the M13 gene-3 minor coat protein (Lee et al., 2004b). The resulting bi-cistronic phagemid contains the light chain fused to an epitope (gD) tag at the C-terminus and heavy chain (V_{H} and C_{H}1) fused to the gene for M13 minor coat protein (p3)C-terminally under the control of the alkaline phosphatase promoter. The first open reading frame encoded a polypeptide consisting of the stII secretion signal followed by the Fab4D5 light chain, with the CDRs replaced by those of 3-7 anti-h VEGF and 4-1 anti-hDR5 scFv′2, followed by a gD-tag epitope. The second open reading frame encoded a fusion polypeptide consisting of the following: the stII secretion signal, the Fab4D5 heavy chain, an amber (TAG) stop codon, a Gly/Ser linker sequence and c-terminal domain of g3 protein (cP3). Expression in E. coli XL-1 Blue co-infected with M13-KO7 resulted in the production of M13 bacteriophage displaying Fab versions of 3-7 and 4-1 scFv′2. Competitive phage ELISAs were used to estimate the affinities of the phage-displayed scFvs and Fabs for hVEGF and hDR5 as IC₅₀ values. The data from the two different formats were in good agreement (data not shown).

To enable display of bH1 Fab on the surface of M13 bacteriophage, plasmid pAP2009 was modified to encode bH1Fab. Versions of the bH1 Fab were used as library templates containing stop codons (TAA) in either the three LC CDRs or the three HC CDRs for the LC and HC library, respectively. Separate heavy chain and light chain alanine and homolog scanning libraries were constructed as previously described (Vajdos et al., J. Mol. Biol. 320:415, 2002). The degeneracy ranged from 1×10⁵ to 1×10⁸ and the actual library size from 6×10⁹ to 4×10¹⁰. The libraries were constructed as described above. Two to three rounds of selection were performed on immobilized targets (VEGF, HER2-ECD, protein L, or anti-gD mIgG) (Vajdos et al., J. Mol. Biol. 320:415, 2002). Target binding clones were screened by phage ELISA for target binding followed by DNA sequencing and sequence alignment to calculate the wild-type/mutation ratios at each position. The ratios from sequence analysis of approximately 100 unique sequences of VEGF and HER2 binding clones were corrected for display and protein folding effect by dividing with ratios calculated from the sequences of more than 100 anti-gD binding clones to yield the F_{wt/mut} values. As only the Fab heavy chain is fused to the phage coat, the phage display of the gD tag, which is fused to the light chain, is indicative of proper folding and association of light chain and heavy chain. Consistently, protein L binding to a non-linear epitope on the light chain of the Fab also resulted in similar wild-type/mutation ratios as gD tag selections. F_{wt/mut} values were converted to ΔΔG using the formula ΔΔG=RTln(K_{a,wt}/K_{a,mut})=RTln(F_{wt/mut}) as described in Vajdos et al. (J. Mol. Biol. 320:415, 2002).

### Expression of Library Binders as Free Human Fab and IgG

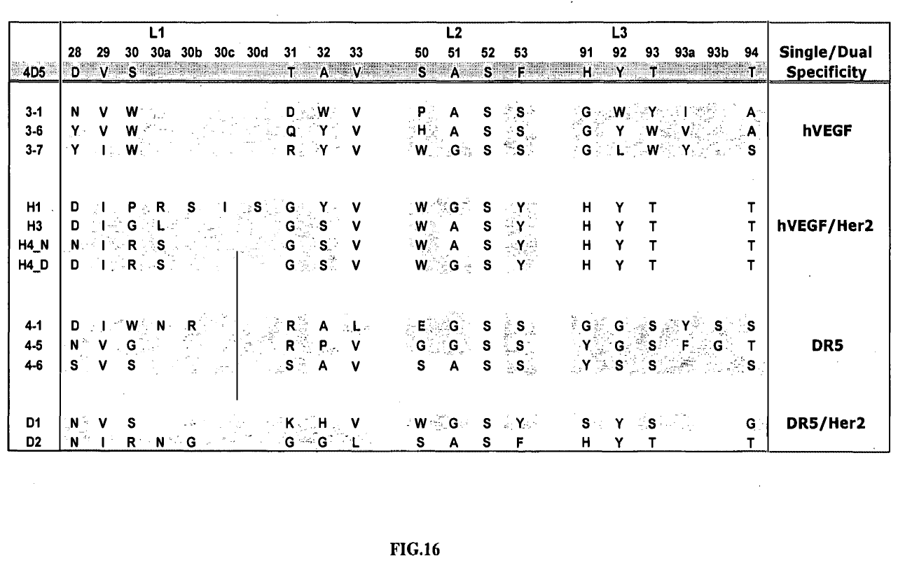

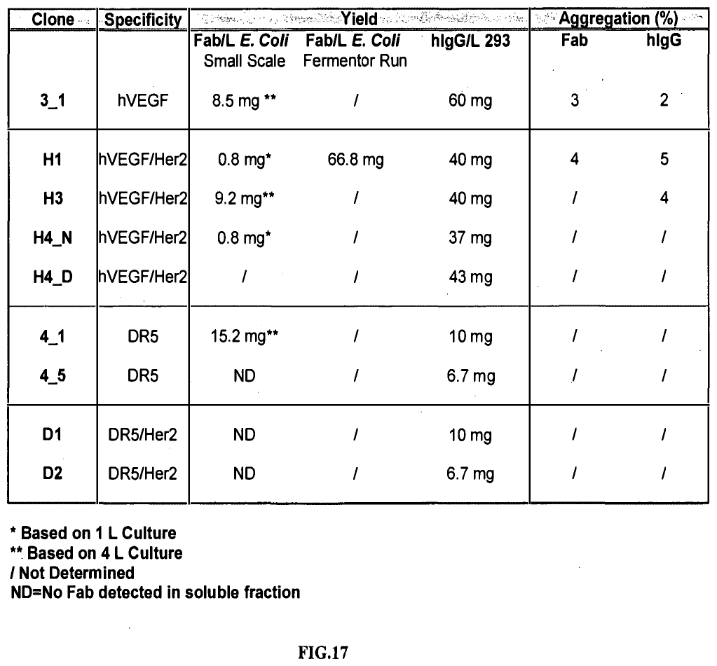

To accurately determine the affinity, specificity and other properties of the antibodies, representative clones from each specificity group exhibiting the highest affinity in the competition ELISA experiments were selected for expression as free Fab and hIgG (FIG. 16). The variable domain of light chain and heavy chain was cloned into a vector previously designed for Fab expression in E. coli or transient human IgG expression in mammalian cells (Lee et al., 2004a). Fab protein was generated by growing the transformed 34B8 E. coli cells in complete C.R.A.P. medium at 30° C. for 26 hours as described (Presta et al., 1997). The hIgGs were expressed by transient transfection of 293 cells and hIgG was purified with protein A affinity chromatography (Fuh et al., J. Biol. Chem. 273:11197, 1998). The 1 L E. coli cultures were purified with protein G affinity chromatography. The columns were washed with PBS and Fab protein was eluted with 100 mM acetic acid and dialyzed against PBS. The 4 L E. coli cultures were purified on a protein A affinity column followed by cation exchange chromatography as previously described (Muller et al., 1998). Protein concentrations were determined spectrophotometrically. The final yield for Fab was typically 0.8-15 mg/l purified from a small-scale shake flask growth. IgG production yield was medium to high at 6.7-60 mg/l in small-scale culture (FIG. 17). The purified proteins were first characterized using size exclusion chromatography and light scattering to ensure that the proteins did not exhibit significant levels of protein aggregation (<5%).

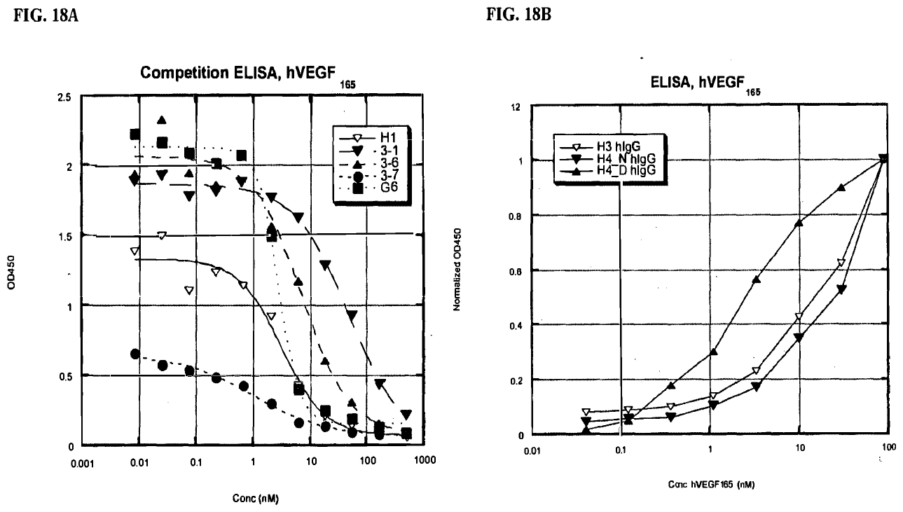

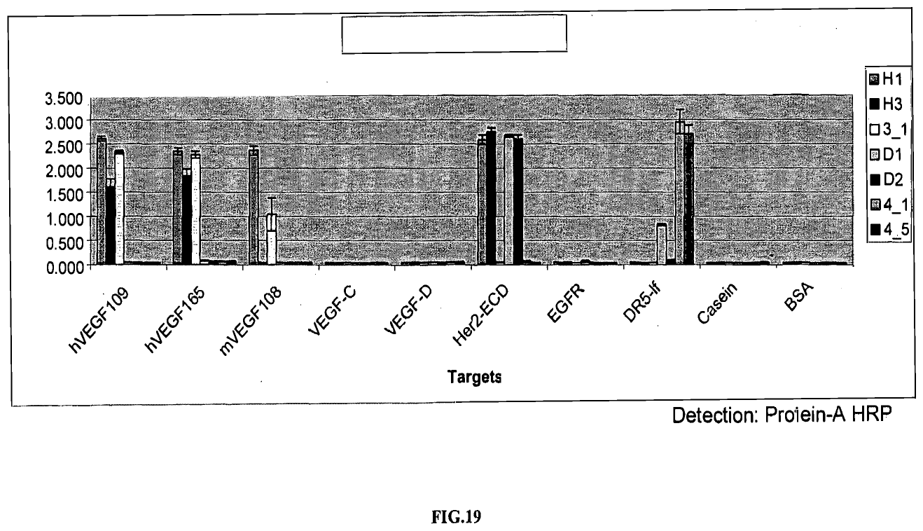

Briefly, the Fabs and hIgGs expressed were screened by ELISA for binding their respective antigen(s). All but one variant were found to bind their cognate antigen(s). Clone 4-6 lost hDR5 binding ability when converted to Fab and hIgG. Selected anti-VEGF clones, raised against the shorter form hVEGF₁₀₉, were tested for binding to hVEGF₁₆₅ using standard ELISA (H3, H4_N, H4_D hIgG), and competitive ELISA (bH1, 3-1, 3-6, 3-7 hIgG). G6 hIgG (Fuh et al., 2006) was used as a positive control (FIG. 18). As expected, all clones bound hVEGF₁₆₅.

To study the extent of protein aggregation selected clones were analyzed by Size-Exclusion chromatography (SEC) followed by Light Scattering (LS) Analysis as purified Fab and IgG. The samples were assayed in PBS at a concentration of 0.5 mg/ml (hIgG) and 1 mg/ml (Fab). A maximum of 5% aggregation was observed for all samples at the given concentration (FIG. 17), which is within range of what we have previously observed for other phage-display derived antibodies. Clones 3-6 and 3-7 did not come out at the expected time point, which suggested these reformatted IgG and Fab exhibit aggregation and or non-specific interaction with the resin (data not shown). These clones were taken out of the set of clones that underwent further analysis.

To rule out cross-reactivity and non-specific binding, we studied binding of selected hIgG at high concentration (100 nM) to a panel of immobilized a panel of protein targets including whole cell lysates, the cognate antigens, and homologues in a standard ELISA assay. In addition to antigen, we immobilized a murine version of hVEGF to test cross-species reactivity of the anti-hVEGF clones. In particular, the panel of proteins was immobilized on Maxisorp plates and blocked with 1% BSA in PBS for 1 hour. The hIgGs (or Fabs) were diluted in PBST to a concentration of 100 or 500 nM and transferred to the coated plates. After a 1 hour incubation, the plates were washed and incubated with HRP-conjugated protein A. The binding signals were developed by addition of TMB substrate for approximately 5 minutes, quenched with 1M H₃PO₄, and read spectrophotometrically at A₄₅₀. The hIgGs tested bound specifically to their antigen(s). Clones bH1 and 3-1 exhibited cross-reactivity to murine VEGF (mVEGF) (FIG. 19).

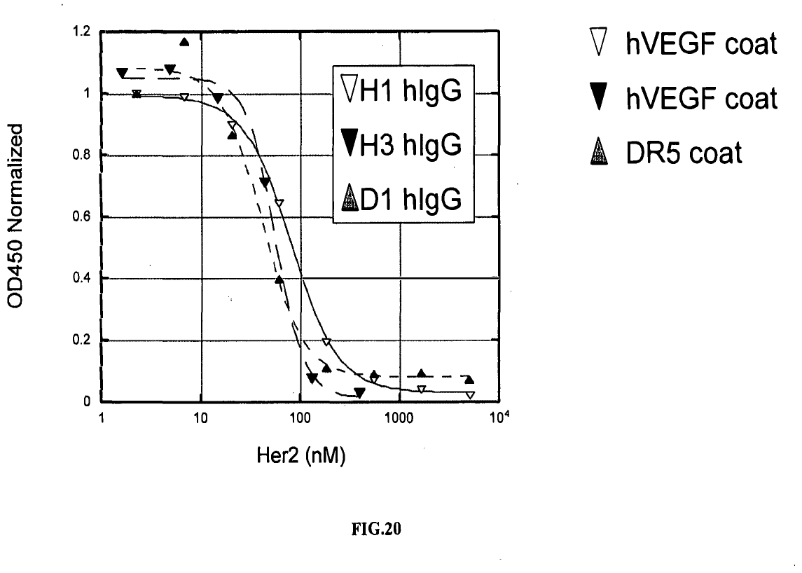

To test whether the bi-specific antibodies bH1, H3 (anti-hVEGF/Her2), and D1 (anti-hDR5/Her2) could simultaneously bind their cognate antigens or if the antigens compete for antibody binding, hVEGF and hDR5 were immobilized at a concentration of 2 μg/ml. A fixed concentration of hIgG was incubated with serial dilutions of Her2-ECD followed by capture of the hIgG on the immobilized antigen. In each case, Her2-ECD binding was found competitive with binding to the other antigens (FIG. 20).

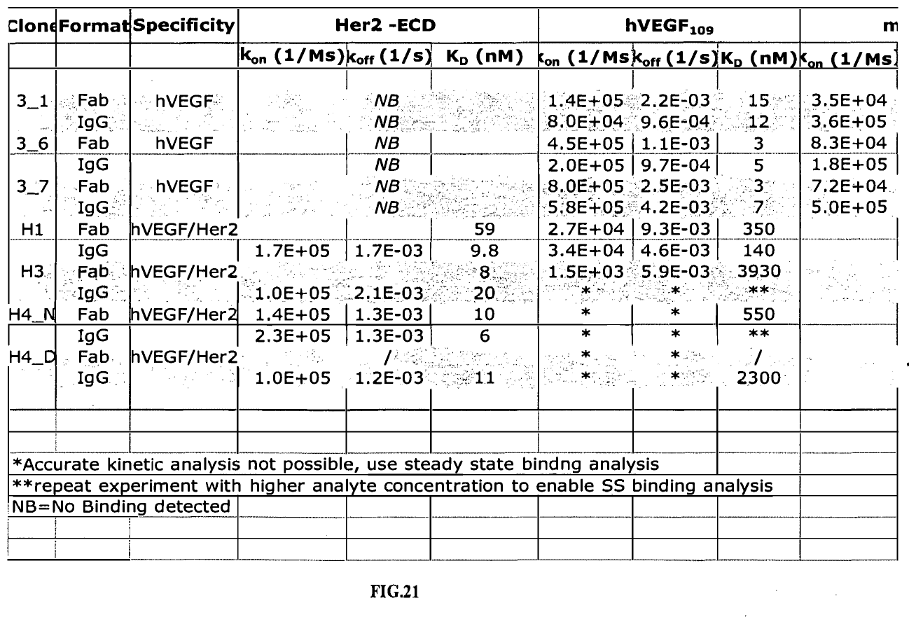

To accurately determine the affinity of IgGs and Fabs (i.e., anti-hVEGF and anti-hVEGF/Her2 Fab and IgG isolated from the libraries) and to study the binding profiles in real time, we used surface plasmon resonance (SPR) assays on a BIAcore™-3000 (BIAcore, Uppsala, Sweden) machine with immobilized hVEGF, mVEGF, DR5, and Her2-ECD CM5 sensor chips at response units (RU) of 40-300 depending on the analyte studied. Immobilization was performed as described (Chen et al., 1999). To minimize avidity effects of the bivalent IgG analytes, a lower density of ligand was targeted on the sensor chip in these cases. Samples of increasing concentrations ranging from a concentration approximately 10-fold below to 10-fold above the estimated K_{D} (based on competition ELISA experiments) were injected at 22-30 μl/minute, and binding responses were corrected by subtraction of RU from a reference flow-cell. In addition, the responses were double referenced to normalize for instrument drift by subtracting RU from ligand-conjugated flow-cell injected with sample buffer (PBS with 0.05% Tween 20). For kinetic analysis of the Fabs, a 1:1 Langmuir binding model of was used to calculate the kₒₙ and k_{off}. When necessary (at high analyte concentrations) a 1:1 Langmuir binding model with mass-transfer limitation was applied. For the IgG analytes, a bivalent analyte binding model with or without mass-transfer limitation was used (BIAcore Evaluation Software 3.2). In the case of H3 hIgG, H4_N Fab, and H4_D hIgG, the fit of responses to the kinetic binding models was not satisfactory. Therefore, steady state binding analysis was applied where the equilibrium response was plotted against analyte concentration. The K_{D} was estimated as the EC₅₀. A summary of the BIAcore binding analysis can be found in FIG. 21. The affinity of the hVEGF binding antibodies 3-1, 3-6 and 3-7 was found to be in the nano molar range. The bi-specific antibodies analyzed (bH1, H3, H4_N, H4_D) showed low micromolar to micromolar affinities for hVEGF. In contrast, the affinities for Her2 ranged from 8-59 nM (Fab).

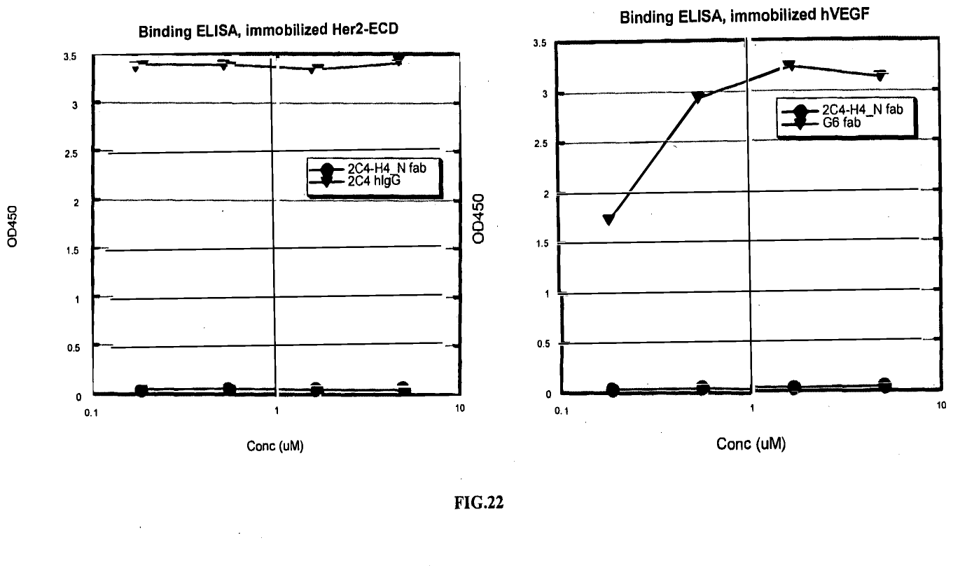

To determine whether the light chain of anti-h VEGF binders bH1, H3, and H4_N could bind hVEGF independent of the sequence of the associated heavy chain, the light chain variable domains were grafted onto the anti-Her2 2C4 Fab by cloning the light chain variable domains into a 2C4 Fab expression vector pJB0524, thus replacing 2C4 light chain variable domain. The Fabs were expressed as previously described. The bH1/2C4 and H3/2C4 chimeric Fabs did not express at detectable levels. The H4_N/2C4 chimeric Fab protein was isolated and tested for binding to hVEGF (bH1 original specificity) and Her2 (bH1, 2C4 original specificity). No binding was detected to hVEGF and Her2 by a standard ELISA binding assay (FIG. 22). The results indicate that the heavy chain of bH1 is required for antigen binding.

### Comparison of Anti-hVEGF Epitopes

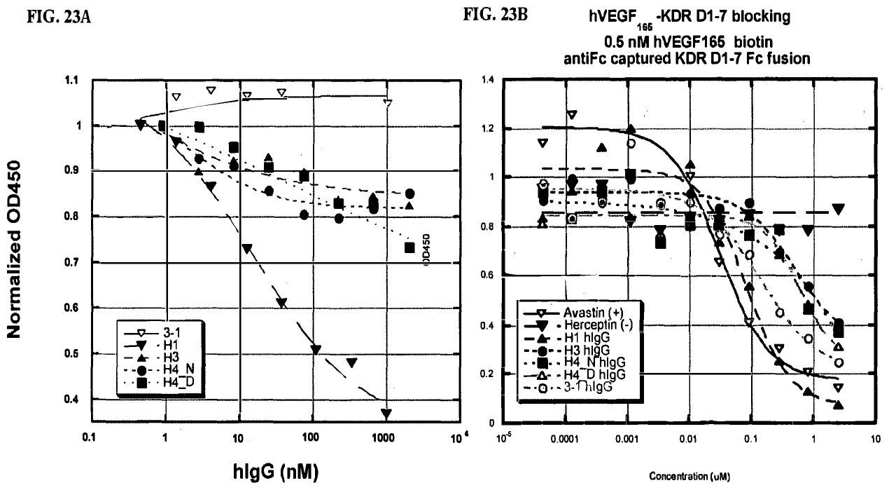

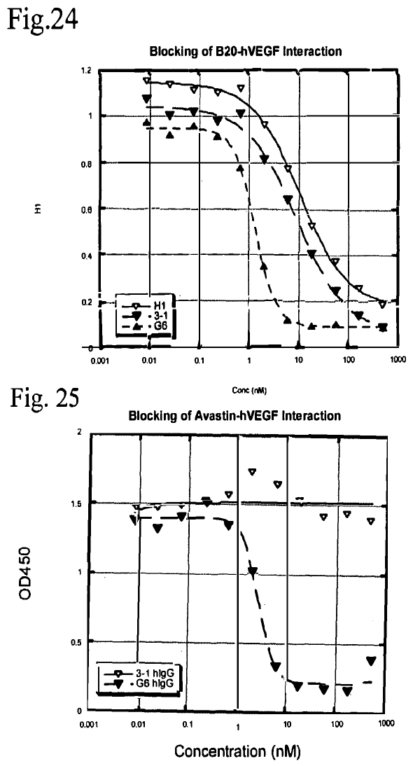

In an attempt to roughly map out the epitopes of the anti-hVEGF antibodies on hVEGF, we studied the ability of these newly isolated anti-VEGF antibody to compete with other hVEGF binding antibodies and VEGF receptors with known binding sites (Fuh et al., 2006; Muller et al., 1998; Wiesmann et al., 1997). The assays were done in a competitive ELISA format where the VEGFR1 (Flt) Domain 1-3 and anti-hVEGF antibodies Avastin® (IgG), B20-4.1 (IgG), G6 (Fab), and KDR Domain 1-7 Fc fusion protein were immobilized on Maxisorp immunoplates at 2 μg/ml. The solution competition binding assay used biotinylated VEGF equilibrated with serial dilutions of purified IgG proteins, and the unbound biotin-VEGF was captured with immobilized Fab or IgG coated on Maxisorb plates and was detected with streptavidin-conjugated HRP (Lee et al., J. Mol. Biol. 340:1073, 2004). Antibodies that block hVEGF from binding other hVEGF-binding antibodies or hVEGF-receptors are likely to share over-lapping epitopes. High concentrations (μM) of the bi-specific hVEGF/Her2-binding antibody, bH1, enabled complete blocking of hVEGF binding to its receptors, VEGFR1 and VEGFR2, suggesting bH1 epitope overlaps sufficiently with VEGFR1 (FIG. 23) and VEGFR2 (FIG. 23). In addition, bH1 blocks hVEGF binding to B20-4.1 (FIG. 24). H3, H4_N, and H4_D also block hVEGF-binding to both receptors, which points to similar epitopes as bH1 (FIG. 23). The incomplete blocking profiles are likely to be a consequence of their relatively low affinity for hVEGF (FIG. 21). 3-1, in contrast, does not block hVEGF from binding VEGFR1, even at the highest concentration (0.5 μM) (FIG. 23). Furthermore, we could not detect 3-1 hIgG blocking of the Avastin® antibody (FIG. 25). However, 3-1 hIgG block hVEGF binding to VEGFR2 (KDR) (FIG. 23) as well as to B20-4.1 (FIG. 24). These results indicate that 3-1 has a unique epitope compared to the other antibodies.

### Example 4. Structure-Function Studies of bH1, Anti-hVEGF/Her2 Bi-Specific Antibody

To elucidate the nature of the bH1 interaction with its two antigens, VEGF and HER2, structural and functional studies was performed. The Herceptin® antibody and bH1 differ in CDR-L1 (V²⁹NTA³² vs. I²⁹PRSISGY³²) and CDR-L2 (S⁵⁰ASF⁵³ vs. W⁵⁰GSF⁵³). The bH1 anti-VEGF/Her2 was chosen as representative for structural characterization based on its dual specific nature and its relatively high affinity for VEGF and Her2. In order to study the functional and structural epitopes on VEGF and Her2, we crystallized the bH1 Fab in complex with VEGF₁₀₉ and the extracellular domain of hHer2 and solved the structures of the two complexes by X-ray crystallography. In addition, we performed alanine and homolog shotgun scanning analysis using combinatorial phage displayed libraries as described (Vajdos et al., 2002).

### bH1 Fab Expression, Purification, Crystallization and Data Collection

The receptor-binding portion of human VEGF, consisting of residues 8-109, was expressed, refolded and purified as described previously (Christinger et al., 1996). Residue 1-624 of the extra cellular domain of Her2 was expressed and purified as previously described (Franklin et al., 2004; Hudziak and Ullrich, 1991).

For large-scale bH1 Fab preparation, whole cell pellet was obtained from a ten liter E. coli fermentation. 220 grams of cell paste was thawed into 1 L PBS, 25 mM EDTA, 1 mM PMSF. The mixture was homogenized and then passed twice through a microfluidizer. The suspension was then centrifuged at 12 k in 250 ml aliquots for 90 minutes. The protein was then loaded onto a Protein G column (25 ml) equilibrated with PBS at 5 ml/minute. The column was washed with equilibration buffer and then eluted with 0.58% acetic acid. The fractions were assayed by SDS PAGE (data not shown). Fractions containing bH1 Fab were pooled and then loaded onto a 50 ml Cation Exchange SP Sepharose column (Pharmacia) equilibrated with 20 mM MES pH 5.5. The Fab was eluted with a sodium chloride gradient in the equilibration buffer. The gradient was linear to 0.5 M NaCl, 20 mM MES pH 5.5. Fractions containing the Fab were identified by SDS-PAGE (data not shown), and pooled. bH1 Fab eluted at a NaCl concentration of approximately 0.5 M. The Fab concentration was determined by measuring the A₂₈₀. The final yield for bH1 Fab was 67 mg/l fermenter growth.

Complexes were obtained by mixing the purified bH1 Fab and VEGF or Her2 ECD in 2:1 molar ratio and purified by size-exclusion chromatography (SP-200, Pharmacia) in 25 mM Tris-HCl. pH 7.5 and 0.3 M sodium chloride for VEGF-Fab complex and with 25 mM Tris-HCl, pH 8 and 0.15 M sodium chloride for the Her2 ECD-Fab complex. The composition of the resulting complexes was verified by SDS PAGE. (Data not shown.) The protein complex was concentrated and used in crystallization trials. Initial hanging-drop experiments using the vapor-diffusion method at 19° C. resulted in small isomorphous crystals from 14 different conditions within 1 week in the case of the bH1-VEGF complex. Crystals of the bH1-Her2 complex appeared in 4 conditions within a week. Crystals from one condition was chosen for further optimization in each case.

For crystallization of bH1 Fab-VEGF (8-109), equal volumes of protein complex solution (10.6 mg/ml protein, 300 mM NaCl, 25 mM Tris-HCl pH 7.5) and crystallization buffer containing 0.15 M D. L Malic Acid pH 7.0, 20% PEG₃₃₅₀ was mixed and equilibrated at 19° C. Large crystals appeared after 24 hours which belonged to space group C222₁ with cell dimensions of a=100.6, b=198.0, c=77.7. The crystal forms contained 1 Fab and 1 VEGF monomer in the asymmetric unit. Prior to data collection the crystals were cryo-protected by transfer between drops containing 5%, 10%, and 15% glycerol in artificial mother liquor, followed by a flash freeze in liquid nitrogen. Data was collected to 2.6 Å at the beam line 5.0.1 of the Advanced Light Source (Berkeley).

Crystals of bH1 Fab-Her2(1-624) was obtained by mixing protein solution (11 mg/ml, 25 mM Tris pH 8 and 150 mM sodium chloride) with crystallization buffer containing 25% w/v PEG₂₀₀₀, 0.1M MES pH 6.5. Crystals appeared after 12 hours that belonged to space group P2₁2₁2₁ with cell dimensions of a=62.3, b=115.1, c=208.2. The crystals contained one Her2-Fab complex in the asymmetric unit. Before data collection the crystals were flash frozen in liquid nitrogen with 20% Ethylene Glycol as cryo-protectant. Data was collected to 2.9 Å at the beam line 5.0.1 of the Advanced Light Source (Berkeley).

### Data Processing, Structure Determination, and Refinement

The data was processed using Denzo and Scalepack (Otwinowski, 1997). The structures of bH1 Fab complexes was solved by Phaser (L. C. Storoni, 2004; Read, 2001). The bH1-Fab-VEGF(8-109) complex was solved using coordinates of VEGF from a previously described VEGF-Fab complex (2FJG) and Fab fragments containing either the variable domains V_{L}/V_{H} or the constant domains C_{H1}/C_{L} of the Herceptin® antibody Fab-Her2 complex (1N8Z). Fragments of Her2 and the variable domain of the Herceptin® antibody Fab from the Her2-Fab complex 1N8Z were used as search models when solving bH1-Her2 structure. The constant domain of the bH1 Fab could not be found using the Herceptin® antibody Fab constant portion as a search model (1N8Z) and had to be docked manually guided by the Herceptin® antibody Fab-Her2 complex structure. Model building and refinement were performed using the programs Refmac (Collaborative Computational Project, 1994) and Coot (Emsley and Cowtan, 2004), respectively. Stereochemical parameters were analyzed using MolProbity (Lovell et al., Proteins 50:437 (2003)). The structures were refined to Rᵥₐₗᵤₑ=0.22 and R_{free}=0.27 for the Fab-VEGF-complex and Rᵥₐₗᵤₑ=0.25 and R_{free}=0.31 for the Fab-Her2-complex. A crystal structure of bH1 Fab in complex with VEGF as well as Her2-ECD was modeled. Some bH1 Fab residues were within 4.5, 4.0, and 3.5 Å of the antigens. The two paratopes (the area on the antibody that makes contact with the antigen) for the two antigens on the same antibody overlap significantly and residues from both light chain and heavy chain are involved with the binding with both antigens. bH1 binds a similar epitope on VEGF as the Avastin® antibody, and bH1 binds Her2 on an essentially identical epitope as the Herceptin® antibody.

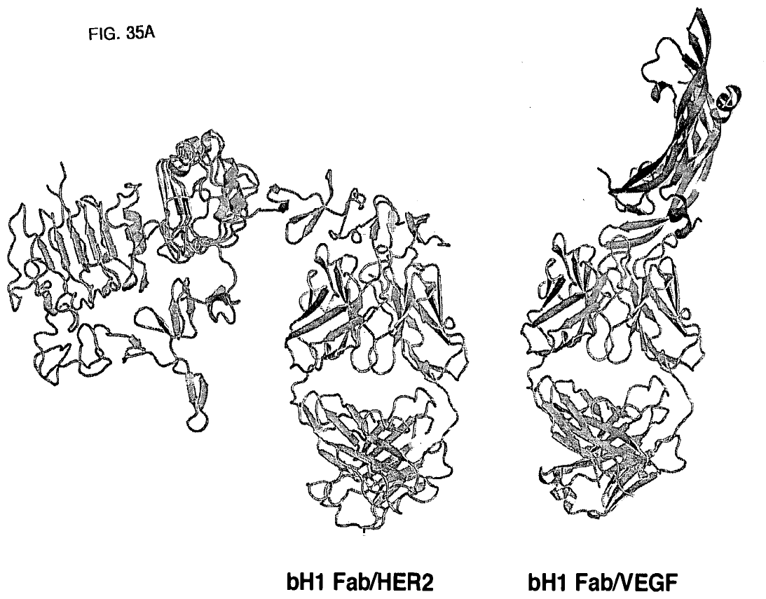

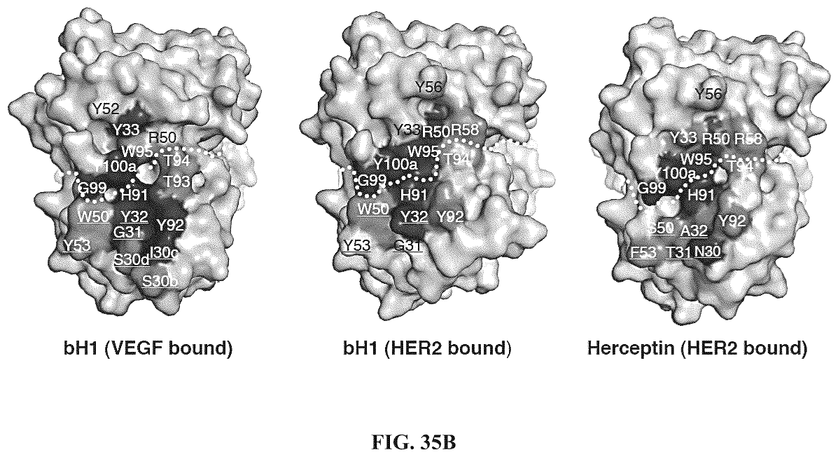

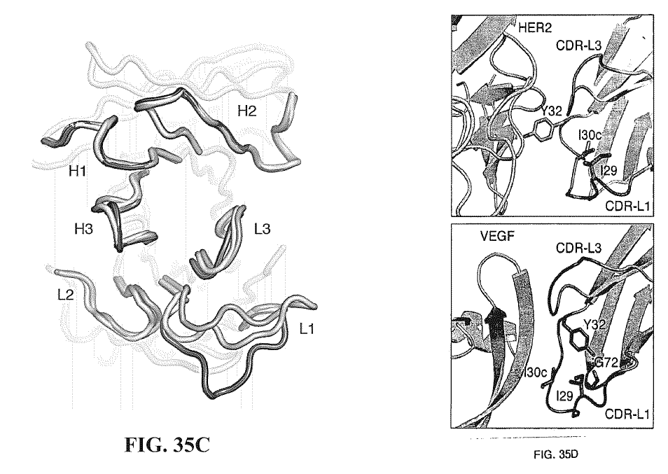

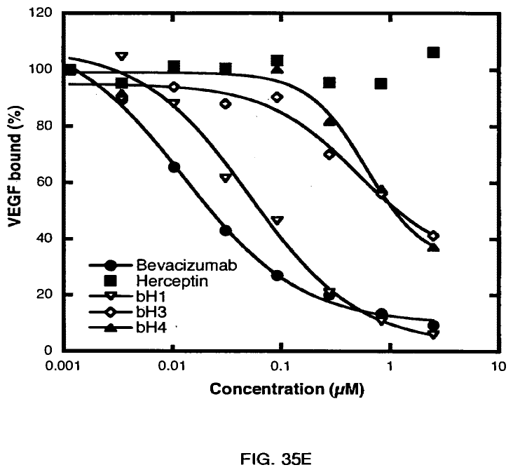

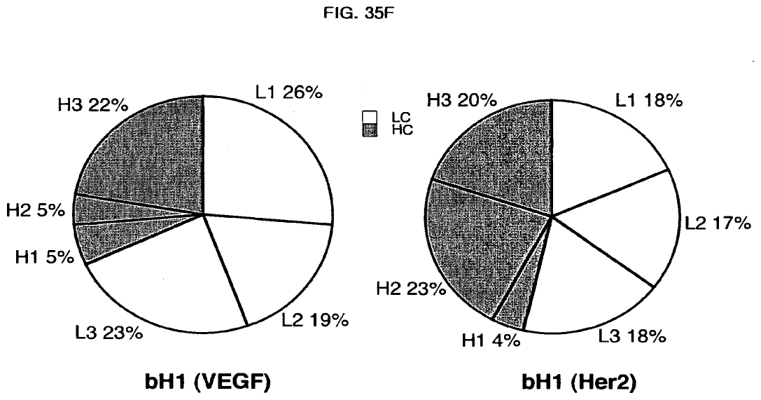

The crystal structures of bH1 Fab bound to the extracellular domain (ECD) of HER2 (residue 1-624) and to the VEGF receptor-binding domain (residue 8-109) were determined at 2.9 Å and 2.6 Å resolutions, respectively (FIG. 35A and Table 3). FIG. 35A shows the bH1 Fab/HER2 crystal structure superimposed with the Herceptin® antibody/HER2 complex, and the crystal structure of the bH1 Fab/VEGF complex.

TABLE 3
Crystallographic Studies

| | bH1 Fab/hVEGF | bH1 Fab/HER2- |
|---|---|---|
| | complex | ECD complex |
| Data Collection Statistics | | |
| Space group | C222₁ | P2₁2₁2₁ |
| Unit Cell (Å) | a = 100.6, b = | a = 62.3, b = |
| | 198.0, c = 77.7 | 115.1, c = 208.2 |
| Beamline, wavelength | ALS 5.0.1 | ALS 5.0.1 |
| Resolution (Å) | 50.0-2.6 | 50.0-2.9 |
| Rsym^{a} | 0.090 (0.66) | 0.095 (0.66) |
| Number of Observations | 151689 | 192951 |
| Unique Reflections | 24705 | 34149 |
| Completeness (%)* | 99.8 (100) | 100 (100) |
| I/σ (I)* | 16.0 (3.0) | 18.5 (2.6) |
| Refinement Statistics | | |
| Content of assymmetric unit | 1/2 VEGF | 1 Her2-ECD |
| | dimer, 1 Fab | monomer, 1 Fab |
| Resolution (Å) | 30.0-2.6 | 30.0-2.9 |
| Reflection used | 22977 | 32277 |
| R Factor^{b}, Rfree | 0.19, 0.25 | 0.22, 0.28 |
| RMS Deviation Bonds (Å) | 0.011 | 0.010 |
| RMS Deviation Angles (°) | 1.3 | 1.3 |
| Ramachandran Statistics | | |
| Favoured Regions (%) | 96.5% | 89.9% |
| Allowed Regions (%) | 99.4% | 97.9% |
| Outliers (%) | 0.6% | 2.1% |
| Number of Residues | 528 | 1017 |
| Numbers of waters | 49 | 0 |
| Number of Sugars | 0 | 2 |
| Number of Ligands/Ions | 1 (Glycerol) | 1 (MES) |

Rsym^{a} = Σ I-<I> ΣI. <I> is the average intensity of symmetry-related observations of a unique reflection.
R Factor^{b} = Σ F0-Fc ΣI F0. Rfree is calculated as R except for 5% of the reflections excluded from all refinements.
*Values in parenthesis denote values of the highest resolution shell.

In the bH1/HER2 complex, the Fab binds to domain IV of HER2 in a manner similar to the Herceptin® antibody (Cho et al., Nature 421:756, 2003); the two complexes superimpose with a root mean square deviation (r.m.s.d.) of Cα positions of 2.3 Å. In the VEGF complex, bH1 recognizes an epitope that overlaps with the binding sites of the VEGF receptors VEGFR1 and VEGFR2 and of other VEGF antibodies (Wiesmann et al., Cell 91:695, 1997; Muller et al., Proc. Natl. Acad. Sci. USA 94:7192, 1997). Consistently, the bH1 blocking of VEGF binding to its receptors was observed (FIG. 35E). For the data shown in FIG. 35E, biotinylated human VEGF₁₆₅ was equilibrated with increasing concentrations of IgG (x-axis). Unbound hVEGF₁₆₅ was captured on immobilized VEGFR2-ECD Fc fusion and detected spectrophotometrically (optical density at 450 nm, y-axis).

As shown in FIG. 35B, the binding sites for VEGF and HER2 on bH1 overlap extensively. Twelve out of the fourteen residues that engage HER2 also contact VEGF. Both binding sites include CDR residues from the HC as well as LC. In the HER2 complex, the LC and HC CDRs contribute approximately equal antigen contact area (53% and 47% respectively) while in the VEGF complex, the LC CDRs constitute nearly 70% of the buried surface (FIG. 35F). The HER2 binding site on the Herceptin® antibody and bH1 are similar and differ only in the CDR-L1 and -L2 regions where the Herceptin® antibody sequence is not conserved in bH1 (FIG. 35B). In FIG. 35B, residues on the bH1 or the Herceptin® antibody Fab surface are shaded according to the extent buried by VEGF or HER2 (dark shading and white lettering >75% buried, intermediate shading and white lettering 50-75% buried, light shading and black lettering 25-49% buried). The underlined residues differ between bH1 and the Herceptin® antibody. The white dotted line depicts the divide of light and heavy chain.

The conformation of bH1 Fab in complex with HER2 is markedly similar to that of the VEGF-bound Fab (r.m.s.d.=0.7 Å, Cα). The CDRs of both bH1 Fab structures superimpose well with each other and with the parent Herceptin® antibody Fv and bH1 Fv (HER2) r.m.s.d.=0.6 Å, the Herceptin® antibody Fv and bH1 Fv (VEGF) r.m.s.d.=1.2 Å. The CDR-L1 is an exception and differs significantly in the two complex structures; the deviation is 4.6 Å (Cα of residues 27-32). FIG. 35C shows that the CDR conformations of bH1 Fab bound to VEGF are markedly similar to HER2-bound bH1 and to the Herceptin® antibody, with exception of the CDR-L1. FIG. 35C is a superposition of the CDR loops as tubes of VEGF-bound bH1 (dark shading), HER2-bound bH1 (white) and HER2-bound the Herceptin® antibody (light shading). The CDR-L1 loop exhibits significantly different conformations in the two bH1 structures (r.m.s.d._{Cα}=4.6 for bH1 residues 27-32) (FIG. 35D). In the HER2 complex, the CDR-L1 is minimally involved in antigen interaction and part of the loop (residues 28-30b) appears flexible. For VEGF binding, the entire loop is well structured and contributes 26% of surface area buried by VEGF.

Two residues in CDR-L1, Ile30c and Tyr32, have different conformations and play different roles in bH1 binding to HER2 or VEGF. In the HER2 complex, the side chain of Ile30c is buried in the hydrophobic core formed by CDR-L1 and CDR-L3 residues. In the VEGF complex, this side chain forms hydrophobic contacts with VEGF. The Cα of Tyr32 is in the same position in the two structures, but its side chain is rotated ˜130 degrees. In the HER2 complex Tyr32 packs against the receptor, while in the VEGF complex the side chain, together with Ile29, form the hydrophobic core and support the conformation of CDR-L1 and CDR-L3. The CDR-L1 conformation is further stabilized by hydrogen bonds between Tyr32 and the LC framework residue Gly72. The structural analysis confirms that Tyr32 is critical for VEGF binding as mutation to either alanine or phenylalanine is not tolerated. Contrary to VEGF binding, mutation of Tyr32 to alanine (back to the Herceptin® antibody residue) is preferred for HER2 binding. Superposition of the two complexes reveals that VEGF would clash with Tyr32 of CDR-L1 in its HER2 bound state (FIG. 35D). In FIG. 35D the side chains of residues Tyr32, Ile30c. Ile29, and Gly72 are shown as sticks. Residues with temperature factors higher than average are shown in darker shading (residues 28-30b). Hydrogen bonding between Tyr32 and Gly72 is illustrated by a dotted line.

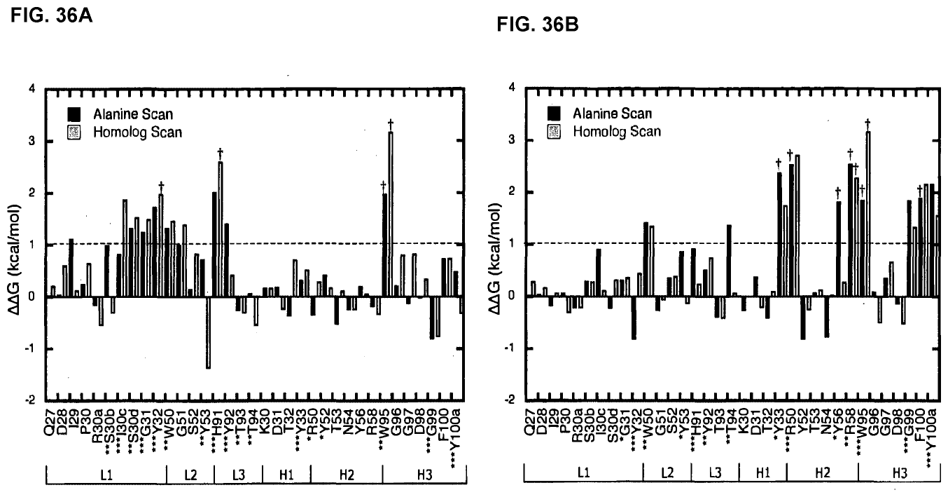

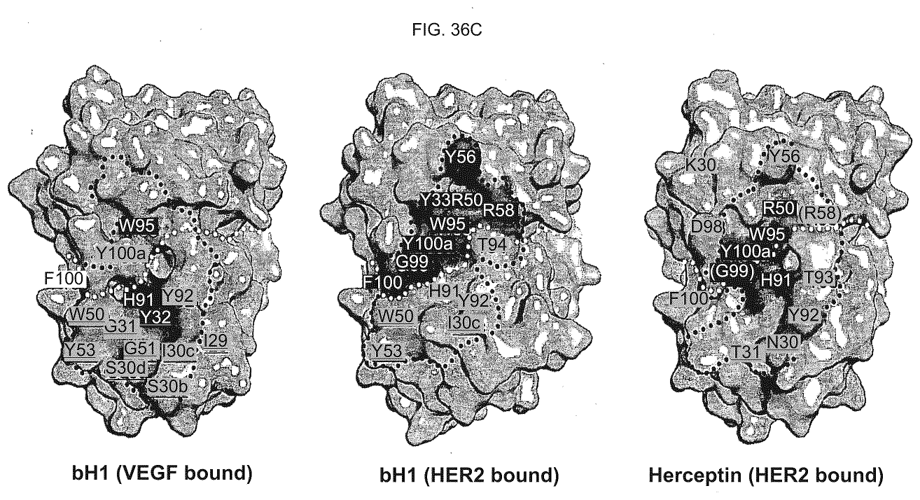

The above results indicate that the capability to rearrange CDR-L1 is necessary for the bi-specificity of bH1. Similar conformational flexibility of CDR-L1 has been shown to play a role in antigen recognition of natural antibodies (Jimenez et al., Proc. Natl. Acad. Sci. USA 100:92, 2003; Mylvaganam et al., J. Mol. Biol. 281:301, 1998). FIGS. 35A-35D and FIG. 36C are generated from the crystal structure coordinates (PDB codes, X, Y, 1N8Z) using PYMOL (DeLano Scientific, San Carlos, CA).

### bH1 Shotgun Scanning

To study the antigen-binding sites of bH1Fab, shotgun scanning combinatorial mutagenesis using phage-displayed Fab libraries was performed (Vajdos et al., J. Mol. Biol. 320:415, 2002; Weiss et al., Proc. Natl. Acad. Sci. USA 97:8950, 2000). Binding selections on the antigens (hVEGF and Her2-ECD) to isolate functional clones followed by DNA sequencing enabled calculations of wild-type/mutant ratios at each varied position (Vajdos et al., 2002). These ratios were then used to determine the contribution of each scanned side-chain to VEGF and Her2 binding. The results enabled mapping of the functional paratope for binding VEGF and Her2.

### bH1 Shotgun Library Design

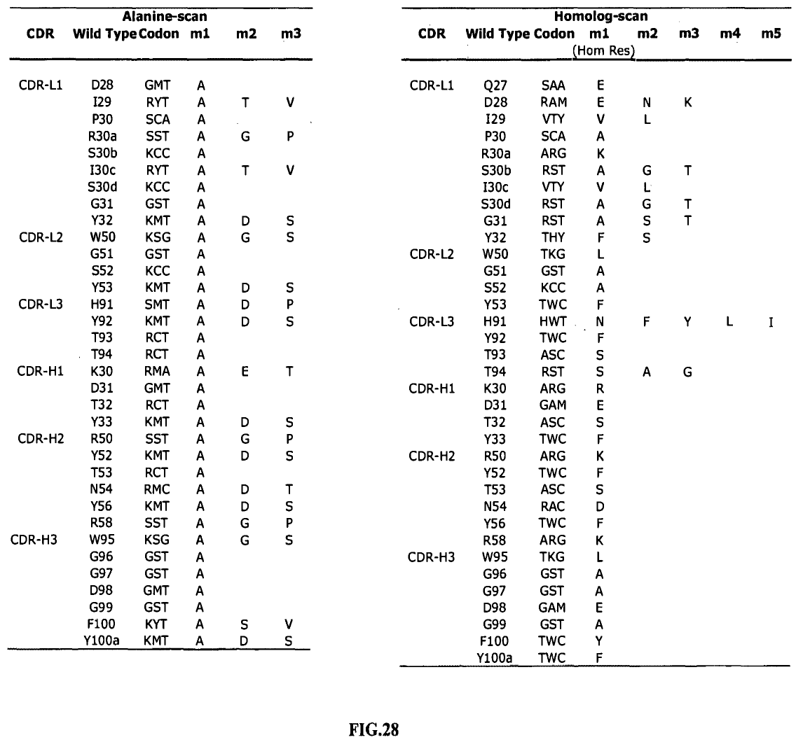

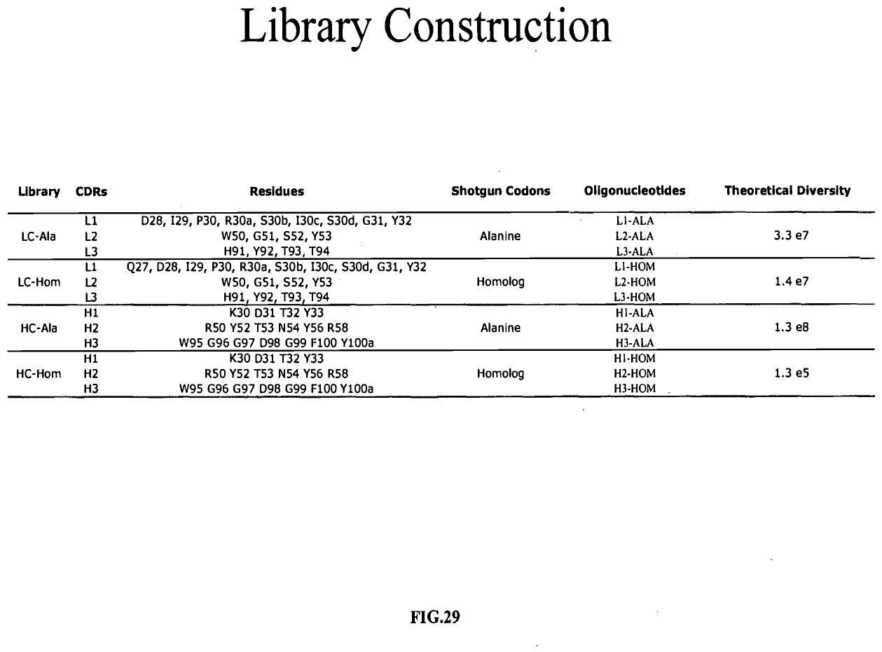

Solvent exposed residues in the CDRs were scanned using phage-displayed libraries in which the wild type residues were allowed to vary as either alanine or wild type (Alanine Scan) or as a homolog residue or wild type (Homolog Scan). The nature of the genetic code required some other substitutions to be included in the library in addition to Wt/Alanine or Wt/Homlog residues (FIG. 28). Separate heavy chain and light chain alanine and homolog scanning libraries were constructed. The libraries are described in FIG. 29. The degeneracy ranged from 1.3×10⁵ to 1.3×10⁸ and the actual library size from 6×10⁹ to 4×10¹⁰.

### Construction of Shotgun Scanning Libraries

As noted above, to enable display of bH1 Fab on the surface of M13 bacteriophage, a previously described plasmid AP2009 designed to display hu4D5Fab on phage fused to the C-terminal domain of the M13 gene-3 minor coat protein, was modified to encode bH1Fab using standard molecular biology techniques. The C-terminus of the light chain contained an epitope (gD) tag. “Stop template” versions of the bH1 Fab was used as library template (Sidhu et al., 2004). The light chain alanine and homolog scanning library had stop codons in CDR-L1, CDR-L2 and CDR-L3 and the heavy chain alanine and homolog libraries contained stop codons in each heavy chain CDR. The libraries were constructed by previously described methods (Sidhu et al., 2004) using Kunkel mutagenesis (Kunkel et al., 1987) on the respective stop templates.

### Library Selection

NUNC 96-well Maxisorp immunoplates were coated with 5 μg/ml capture target (hVEGF₁₀₉, Her2-ECD or anti-gD mIgG) and blocked with 1% BSA (w/v) in PBS. Phage from the above-described libraries were propagated with KO7 helper phage (NEB) as described (Lee et al., 2004a). The library phage solutions were added to the coated plates at a concentration of 10¹³ phage particles/ml and incubated for 1-2 hours at RT. The plates were washed 8 times with PBST and followed by elution of bound phage with 0.1 M HCl for 30 minutes. Enrichment after each round of selection was determined as described previously. After 2 rounds of target selection, 50-1000 fold enrichment was observed for all libraries except LC-Ala and LC-Hom sorted on hVEGF, which showed 5-10-fold enrichment. A number of random clones from each library exhibiting 50-1000-fold enrichment was selected for sequencing as described (Sidhu et al., 2004). Library LC-Ala was screened for hVEGF binding in phage ELISA (Sidhu et al., 2000). Clones that exhibited hVEGF ELISA signals at least two-fold over signals on a control plates coated with BSA were selected for sequencing. The LC-Hom library was subjected to 1 additional round of selection on hVEGF followed by phage ELISA screening and sequencing of VEGF-binding clones.

### DNA Sequence Analysis

The high quality sequences from each library from the different target selections were translated and aligned (Data not shown). The number of sequences from each library subject to analysis is summarized in Table 4 below.

TABLE 4
Number of Sequences Analyzed

| Library | Total Sequences |
|---|---|
| LCA-V2b | 51 |
| LCH-V3 | 79 |
| LCA-H2 | 97 |
| LCH-H2 | 50 |
| LCA-gD | 112 |
| LCH-gD | 120 |
| LCA-pL | 60 |
| LCH-pL | 65 |
| HCA-V2 | 100 |
| HCH-V2 | 96 |
| HCA-H2 | 81 |
| HCH-H2 | 96 |
| HCA-gD | 105 |
| HCH-gD | 105 |
| HCA-pl | 102 |
| HCH-pl | 99 |

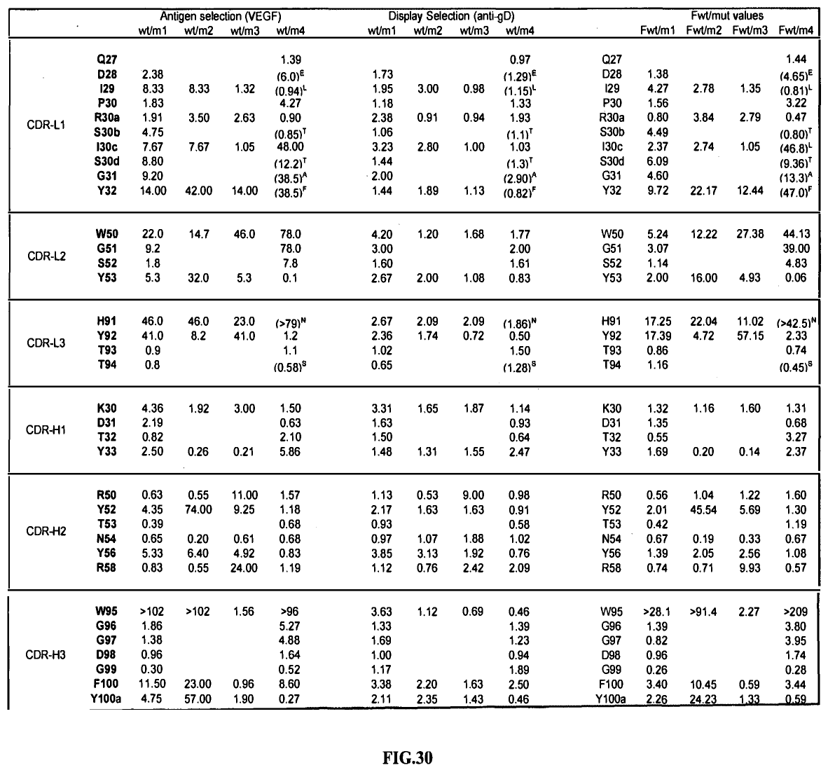

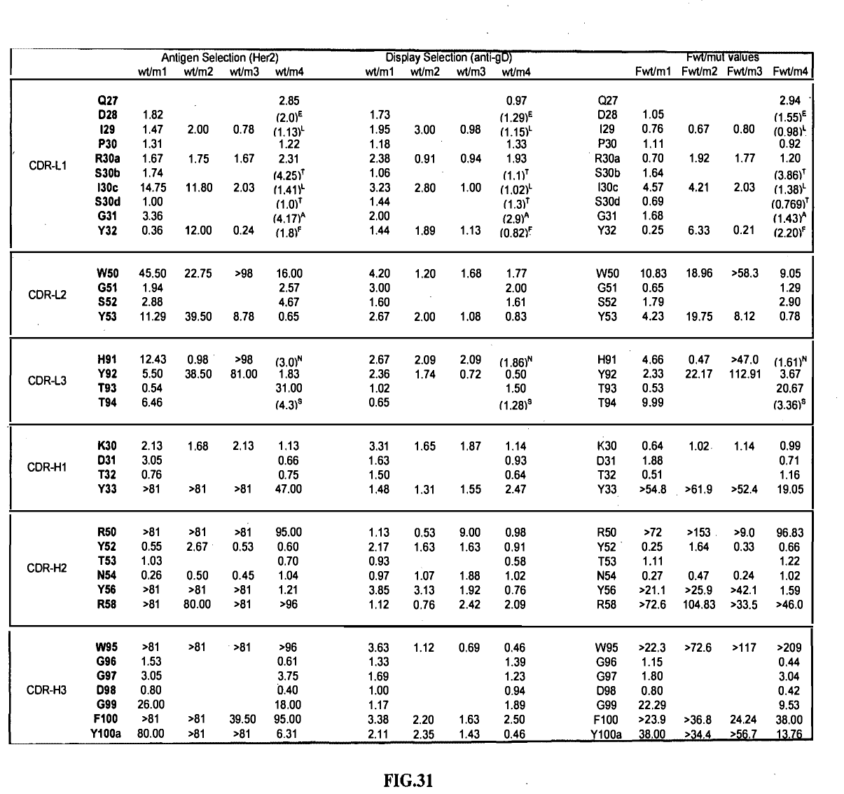

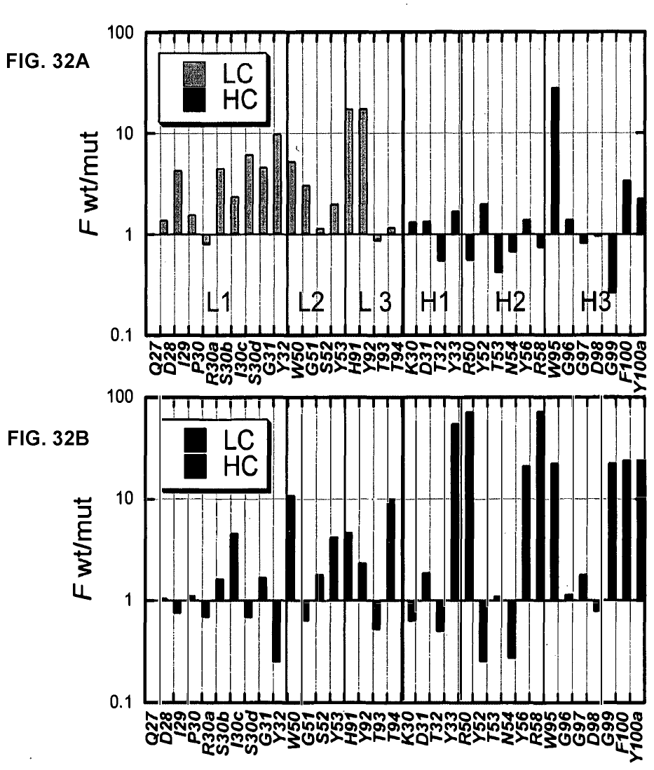

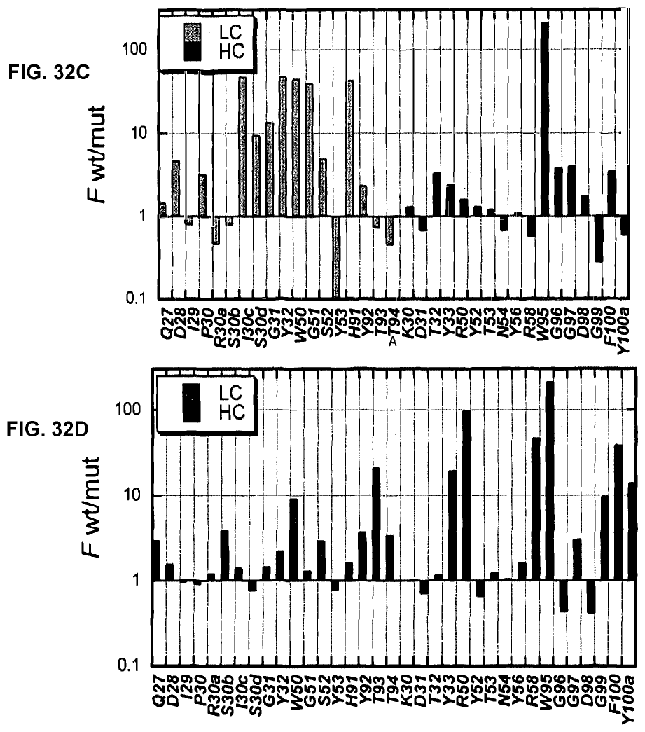

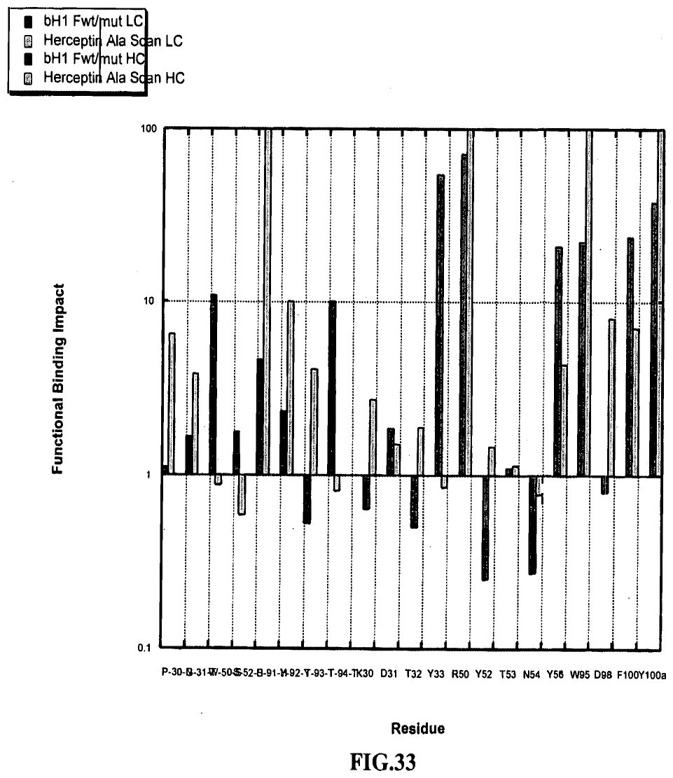

The Wt/Mut ratios were calculated at each varied position (FIG. 30 and FIG. 31) thus allowing calculation of the F_{wt/mut} values as listed (FIG. 30 and FIG. 31) which are corrected for display by division of the ratios from target selection by those from the display selection as described (Vajdos et al., 2002). A F_{wt/mut} value greater than 1 indicates that Wt is preferred at this position and F_{wt/mut} smaller than 1 indicates the mutation is preferred. F_{wt/mut}>5 indicate its important role in antigen binding. The importance of each scanned CDR residue is illustrated in FIGS. 32A-32D. The result demonstrates that residues from both heavy chain and light chain contribute energetically to the binding of both antigen (Her2 and hVEGF) binding. The impact of bH1 light chain and heavy chain residues on Her2 binding was compared to that of its parent antibody hu4D5 (Kelley and O'Connell, 1993) (FIG. 33).

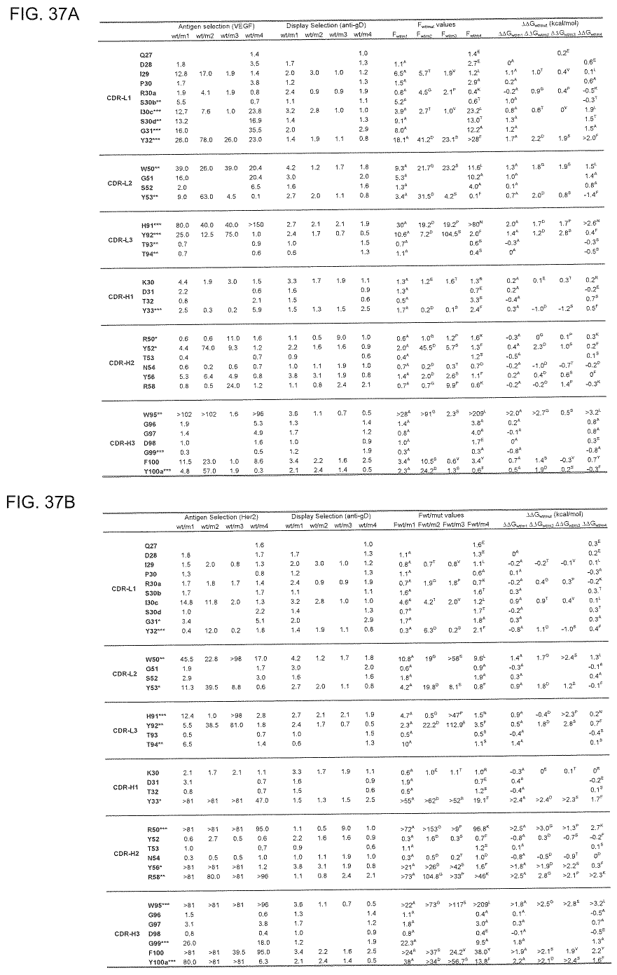

FIG. 37A and FIG. 37B show shotgun alanine- and homolog scanning of bH1 Fab for binding to VEGF and HER2. The effects of mutation of alanine (m1), or additional mutations (m2, m3; due to limitations of shotgun-alanine codons), or to a homologous amino acid (m4) are calculated as the ratio of occurrence of wild-type and mutants (wt/mut) among the clones binding to human VEGF (FIG. 37A) or HER2 (FIG. 37B). In cases where only the wild-type residue appeared, the ratios are shown as larger than “>” the wild-type count. The identity of the amino acid substitutions (m1-m4) is shown as superscripts on the F values. When the wild-type residue is alanine, it was substituted by glycine (m1). The “*” indicates the extent of the bH1 residues that are buried upon VEGF or HER2 complex formation (*25-49% of accessible area buried, **50-75% of accessible area buried, ***greater than or equal to 75% of accessible area buried).

The residues that contribute significantly to the energetic interactions make up the functional paratopes, which constitute a subset of the structural binding sites. In contrast to the extensive overlap between the sites of antigen contact the two functional paratopes show limited overlap (FIGS. 36A-36C). In particular, based on shotgun scanning mutagenesis, the ΔΔG values (y-axis, kcal/mol) are plotted for each mutation to alanine (black bar) or a homologous amino acid (white bar) for VEGF (FIG. 36A) or HER2 (FIG. 36B) binding. The “†” represents a lower limit, as mutations were not observed at this position. The “*” indicates the extent of the bH1 residue surface area buried upon VEGF or HER2 complex formation. (*25-49% buried, **50-75%, ***>75%). The VEGF binding interaction is mediated primarily by the LC CDRs with Tyr32 of CDR-L1 and His91 of CDR-L3 as the core hot spot (ΔΔG_{wt/ala}>1.5 kcal/mol). HER2 binding is mainly contributed by HC CDRs. FIG. 36C shows crystal structures where the bH1 and the Herceptin® antibody residues are shaded on the Fab surface based on their functional importance (dark shading and white lettering. ΔΔG≥1.5 kcal/mol; intermediate shading and black lettering. 1≤ΔΔG<1.5 kcal/mol; light shading and black lettering, 0.5≤ΔΔG<1 kcal/mol of alanine mutation). The black dotted line outlines the contact area as in FIG. 35B. The white dotted line depicts the divide of light and heavy chain.

For VEGF binding and HER2 binding, the functional paratope residues are distributed across HC and LC signifying the synergy of the two chains. Trp95 of CDR-H3 is the only common hot spot residue for the two interactions (ΔΔG_{wt/ala}>1.5 kcal/mol). As noted above, the VEGF binding interaction is mediated primarily by the LC CDRs while HER2 binding is dominated by HC CDRs. Compared to the Herceptin® antibody, bH1 with weaker HER2 binding affinity (300 fold) maintains the same core hot spot residues for HER2 binding (Arg50. Trp95, and Tyr100a) while the importance of peripheral residues is redistributed (FIG. 36C). Overall, most of the important side chains in heavy chain contributing hu4D5/Her2 binding are still important for bH1/Her2 binding (ΔΔG>1.5 kcal/mol). There are some changes. Light chain residues have more shuffling in contributions—some residues became less important and some more important. Overall, the functional sites are part of the structural interface from the crystal structure of the bH1-VEGF and bH1-Her2 complexes.

In short, the interaction of bH1 with the two structurally unrelated large proteins is characterized by the engagement of a distinct set of bH1 residues in the energetic interaction with each antigen. While most of the two extensively overlapping binding sites for the two different antigens exhibit a single conformation, the flexibility of one CDR loop (L1) facilitates the accommodation of both HER2 and VEGF. The mechanism is reminiscent of the molecular versatility observed in multi-specific antibodies binding unrelated small haptens or peptides. Previous studies describe multi-specificity mediated either by differential positioning of the small ligands at spatially distinct regions of a single antibody conformation (Sethi et al., Immunity 24:429, 2006) or by multiple pre-existing conformations of the antigen binding site (James et al., Science 299:1362, 2003). The versatility of antibody molecules in antigen recognition is further highlighted by how limited LC mutations can give rise to antibodies that bind two unrelated protein antigens.

### bH1 Affinity Maturation

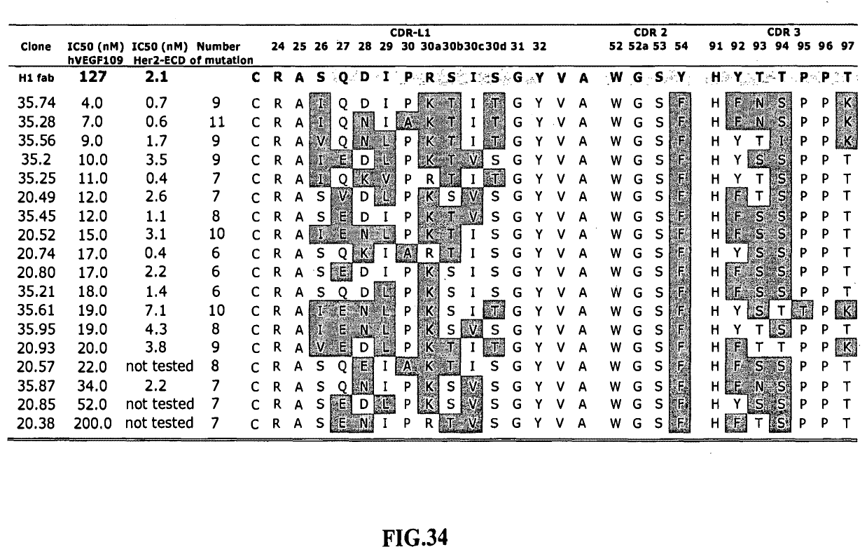

In an attempt to investigate whether the VEGF-binding affinity of bH1 could be increased by optimization of the light chain sequence before the structural and functional results became available, a library was constructed where the CDR residues at highly solvent-accessible positions based on the crystal structure of h4D5⁴² Fab (Eigenbrot et al., 2001), which is assumed to closely resemble bH1 Fab, were diversified. Targeted residues were allowed to vary as either wild type or a few homologous residues (FIG. 29). The library was constructed as described in section “Construction of shotgun scanning libraries.” A solution-based selection method was used to select for higher affinity VEGF-binders as described. Two rounds of solution-based selection were performed. The stringency was increased in each round of selection by decreasing the concentration of biotinylated VEGF from 50 nM in the first round to 20 nM in the second round. 138 clones were sequenced from the last round of selection. Most clones were found to be unique. A high-throughput ELISA assay with immobilized VEGF (8-109), anti-gD antibody, and Her2-ECD was used to identify clones that bound to VEGF, Her2-ECD, and anti-gD mIgG but not to BSA. The VEGF-ELISA binding signals were normalized by the anti-gD ELISA signals to estimate the relative affinity of the VEGF binding clones. Clones with high VEGF/anti-gD ratios were selected for further characterization. The affinity of the selected clones for VEGF and Her2 was estimated by competition ELISA as phage-displayed Fabs as previously described. The bH1 variants show improved VEGF binding-affinity compared to the parent bH1 clone. Interestingly, some clones have slightly improved IC₅₀ values for Her2 binding even though that affinity-based selection for Her2 was not performed. This shows that it is possible to affinity mature the bH1 clone for VEGF binding without affecting Her2 binding ability significantly. There are some VEGF-affinity improved clones that showed reduced Her2 binding affinity compared to the parent bH1 clone. This result indicates that the light chain actively contributes to the binding ability of bH1 to Her2 despite the fact that heavy chain is the main contributor to the binding energy based on the bH1-Her2 complex structure and shotgun alanine scanning analysis. The sequences and IC₅₀ values of the characterized clones are summarized in FIG. 34. The finding that most sequences were unique suggests that the light chain sequence of these variants is not yet fully optimized for VEGF binding and that it is possible to further affinity-improve bH1 clone by additional rounds of selection.

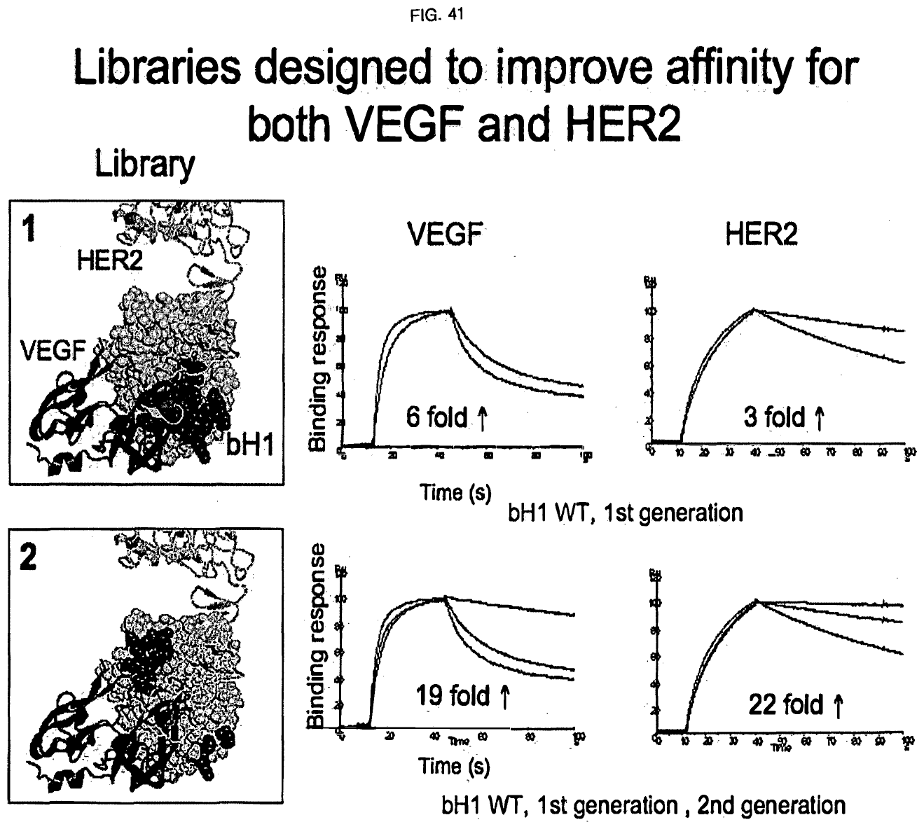

As shown in FIG. 41, significant affinity improvement of a single Fab for two antigens is achievable and generally applicable. For instance, the K_{D} of bH1 for VEGF was increased from 300 to 16 nM and the K_{D} of bH1 for HER2 was increased from 26 to 1.2 nM.

### Example 5. Analysis of IgG Activity in Cell Assays

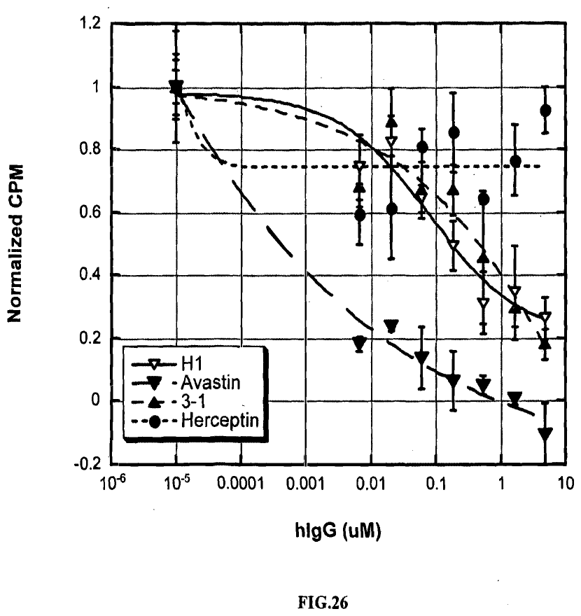

To check whether the two highest affinity anti-hVEGF antibodies, bH1 and 3-1, could inhibit hVEGF₁₆₅ induced proliferation of human umbilical vein endothelial (HUVEC) cells they were tested in a proliferation assay. Human umbilical vein endothelial cells (HUVEC) (Cambrex, East Rutherford, NJ) were grown and assayed as described (Fuh et al., J. Biol. Chem. 273:11197, 1998). Approximately 4000 HUVECs were plated in each well of the 96-well cell culture plate and incubated in Dulbecco's modified Eagle's/F-12 medium (1:1) supplemented with 1.0% (v/v) fetal bovine serum (assay medium) for 18 hours. Fresh assay medium with fixed amounts of VEGF (0.2 nM final concentration), which was first titrated as a level of VEGF that can stimulate submaximal DNA synthesis, and increasing concentrations of anti-VEGF antibodies (e.g., bH1) were then added to the cells. After incubation at 37° C. for 18 hours, cells were pulsed with 0.5 μCi/well of [³H]Thymidine for 24 hours and harvested for counting with TopCount Microplate Scintillation counter. The results demonstrate that both 3-1 and bH1 can inhibit VEGF-induced growth of HUVEC cells by preventing hVEGF induced signaling and subsequent proliferation. The Avastin® antibody (anti-VEGF) was used as a positive control and the Herceptin® antibody as a negative control (FIG. 26).

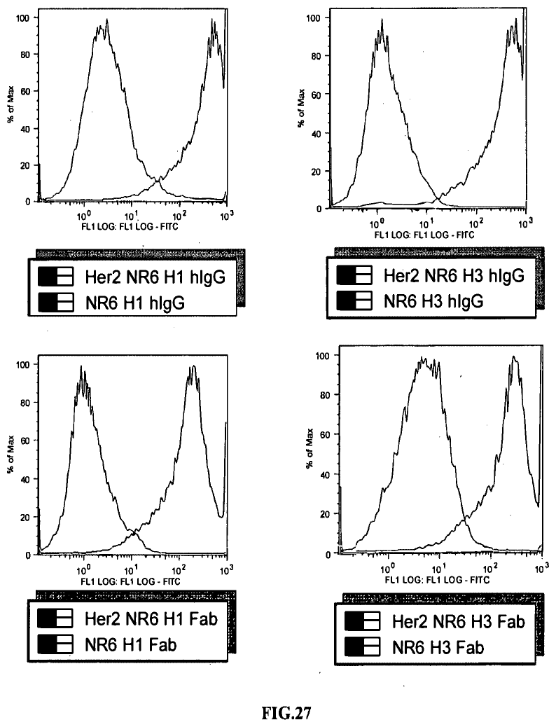

To study binding of bi-specific anti-Her2/VEGF antibodies to Her2 expressed on mammalian cells, the binding of bH1 and H3 antibodies to NR6 fibroblast cells over-expressing Her2 (NR6-Her2) was studied by Flow Cytometry. One million NR6-Her2 cells were incubated with 100 μg/ml Fab and IgG for 1 hour, followed by incubation with an Alexa488-conjugated murine anti-human IgG antibody for 1 hour. As negative controls, Fab and IgG binding to non-expressing NR6 cells was studied. As demonstrated in FIG. 27, bH1 and H3 bind specifically to Her2 on NR6 cells as Fab and as IgG.

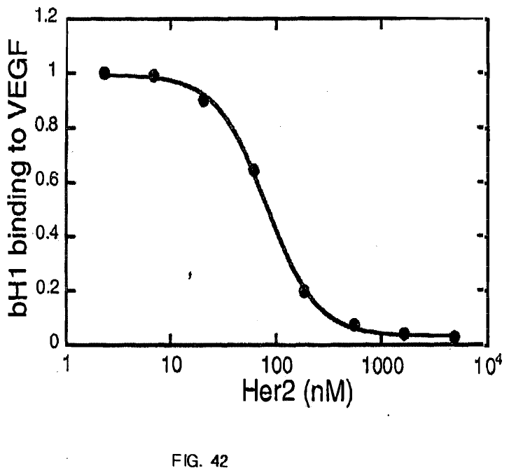

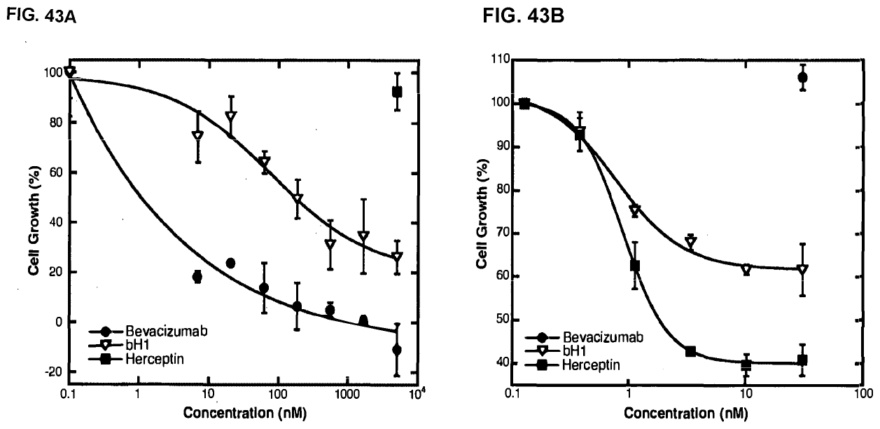

FIG. 42 shows the results of competitive binding experiments for bH1 to VEGF or HER2. bH1 inhibited VEGF induced proliferation of human umbilical vein endothelial cells (HUVEC) with an IC₅₀ of 200 nM, which is consistent with its affinity of 300 nM, and the proliferation of HER2-expressing breast cancer cell line BT474 after 5-day incubation, albeit with lower efficiency than the Herceptin® antibody due to its reduced affinity (FIG. 43A and FIG. 43B). The Herceptin® IgG antibody and bevacizumab (anti-VEGF) served as controls. In particular, breast cancer cells BT474 were cultured in RPMI media supplemented with 10% FBS. For the assays, 10⁴ cells were plated per well in a 96-well plate and incubated overnight (18 hours) at 37° C. Increasing concentrations of antibody were added to the cells. The cells were then incubated at 37° C. for five days, followed by addition of 10% AlamarBlue (Biosource International, Camarillo, CA) according to the manufacturer's instructions. The antibody-dependent inhibition of proliferation of the HER2 expressing cells was determined by measuring the fluorescent signal.

The above results indicate the potential of the bi-functional bH1, or its affinity improved variants, to inhibit two mechanisms that are important for tumor growth in vivo.

### REFERENCES CITED

Chen, Y., C. Wiesmann, G. Fuh, B. Li, H. W. Christinger, P. McKay, A. M. de Vos, and H. B. Lowman, 1999, Selection and analysis of an optimized anti-VEGF antibody: crystal structure of an affinity-matured Fab in complex with antigen: J Mol Biol, v. 293, p. 865-81.
Cho, H. S., K. Mason, K. X. Ramyar, A. M. Stanley, S. B. Gabelli, D. W. Denney, Jr., and D. J. Leahy, 2003, Structure of the extracellular region of HER2 alone and in complex with the Herceptin Fab: Nature, v. 421, p. 756-60.
Christinger, H. W., Y. A. Muller, L. T. Berleau, B. A. Keyt, B. C. Cunningham, N. Ferrara, and A. M. de Vos, 1996, Crystallization of the receptor binding domain of vascular endothelial growth factor: Proteins, v. 26, p. 353-7.
Collaborative Computational Project, N., 1994: Acta Crystallogr. Section D. Biol. Crystallogr., v. 50, p. 760-763.
Emsley, P., and K. Cowtan, 2004, Coot: model-building tools for molecular graphics: Acta Crystallogr D Biol Crystallogr, v. 60, p. 2126-32.
Fellouse, F. A., B. Li, D. M. Compaan, A. A. Peden, S. G. Hymowitz, and S. S. Sidhu, 2005, Molecular recognition by a binary code: J Mol Biol, v. 348, p. 1153-62.
Franklin, M. C., K. D. Carey, F. F. Vajdos, D. J. Leahy, A. M. de Vos, and M. X. Sliwkowski, 2004, Insights into ErbB signaling from the structure of the ErbB2-pertuzumab complex: Cancer Cell, v. 5, p. 317-28.
Fuh, G., B. Li, C. Crowley, B. Cunningham, and J. A. Wells, 1998, Requirements for binding and signaling of the kinase domain receptor for vascular endothelial growth factor: J Biol Chem, v. 273, p. 11197-204.
Fuh, G., P. Wu, W. C. Liang, M. Ultsch, C. V. Lee, B. Moffat, and C. Wiesmann, 2006, Structure-function studies of two synthetic anti-vascular endothelial growth factor Fabs and comparison with the Avastin Fab: J Biol Chem, v. 281, p. 6625-31.
Hudziak, R. M., and A. Ullrich, 1991, Cell transformation potential of a HER2 transmembrane domain deletion mutant retained in the endoplasmic reticulum: J Biol Chem, v. 266, p. 24109-15.
Kelley, R. F., and M. P. O'Connell, 1993, Thermodynamic analysis of an antibody functional epitope: Biochemistry, v. 32, p. 6828-35.
Kunkel, T. A., J. D. Roberts, and R. A. Zakour, 1987, Rapid and efficient site-specific mutagenesis without phenotypic selection: Methods Enzymol, v. 154, p. 367-82.
L. C. Storoni, A. J. M, a. R. J. R., 2004, Likelihood-enhanced fast rotation functions.: Acta Cryst., p. 432-438.
Lasky, L. A., and D. J. Dowbenko, 1984, DNA sequence analysis of the type-common glycoprotein-D genes of herpes simplex virus types 1 and 2: DNA, v. 3, p. 23-9.
Lee, C. V., W. C. Liang, M. S. Dennis, C. Eigenbrot, S. S. Sidhu, and G. Fuh, 2004a, High-affinity human antibodies from phage-displayed synthetic Fab libraries with a single framework scaffold: J Mol Biol, v. 340, p. 1073-93.
Lee, C. V., S. S. Sidhu, and G. Fuh, 2004b, Bivalent antibody phage display mimics natural immunoglobulin: J Immunol Methods, v. 284, p. 119-32.
Liang, W. C., X. Wu, F. V. Peale, C. V. Lee, Y. G. Meng, J. Gutierrez, L. Fu, A. K. Malik, H. P. Gerber, N. Ferrara, and G. Fuh, 2006, Cross-species vascular endothelial growth factor (VEGF)-blocking antibodies completely inhibit the growth of human tumor xenografts and measure the contribution of stromal VEGF: J Biol Chem, v. 281, p. 951-61.
Lowman, H. B., S. H. Bass, N. Simpson, and J. A. Wells, 1991, Selecting high-affinity binding proteins by monovalent phage display: Biochemistry, v. 30, p. 10832-8.
Muller, Y. A., Y. Chen, H. W. Christinger, B. Li, B. C. Cunningham, H. B. Lowman, and A. M. de Vos, 1998, VEGF and the Fab fragment of a humanized neutralizing antibody: crystal structure of the complex at 2.4 A resolution and mutational analysis of the interface: Structure, v. 6, p. 1153-67.
Otwinowski, Z., and Minor, W., 1997, Processing of X-ray diffraction data collected in oscillation mode.: Methods Enzymol., v. 276, p. 307-326.
Presta, L. G., H. Chen, S. J. O'Connor, V. Chisholm, Y. G. Meng, L. Krummen, M. Winkler, and N. Ferrara, 1997, Humanization of an anti-vascular endothelial growth factor monoclonal antibody for the therapy of solid tumors and other disorders: Cancer Res, v. 57, p. 4593-9.
Read, R. J., 2001, Pushing the boundaries of molecular replacement with maximum likelihood.: Acta Cryst., v. D57, p. 1373-1382.
Sidhu, S. S., B. Li, Y. Chen, F. A. Fellouse, C. Eigenbrot, and G. Fuh, 2004, Phage-displayed antibody libraries of synthetic heavy chain complementarity determining regions: J Mol Biol, v. 338, p. 299-310.
Sidhu, S. S., H. B. Lowman, B. C. Cunningham, and J. A. Wells, 2000, Phage display for selection of novel binding peptides: Methods Enzymol, v. 328, p. 333-63.
Vajdos, F. F., C. W. Adams, T. N. Breece, L. G. Presta, A. M. de Vos, and S. S. Sidhu, 2002, Comprehensive functional maps of the antigen-binding site of an anti-ErbB2 antibody obtained with shotgun scanning mutagenesis: J Mol Biol, v. 320, p. 415-28.
Wiesmann, C., G. Fuh, H. W. Christinger, C. Eigenbrot, J. A. Wells, and A. M. de Vos, 1997, Crystal structure at 1.7 A resolution of VEGF in complex with domain 2 of the Flt-1 receptor: Cell, v. 91, p. 695-704.

All patents, patent applications, patent application publications, and other publications cited or referred to in this specification are herein incorporated by reference to the same extent as if each independent patent, patent application, patent application publication or publication was specifically and individually indicated to be incorporated by reference.

What is claimed is:

1-43. (canceled)

44. A method of treating a tumor in a subject, the method comprising administering to the subject an antibody capable of binding to a first epitope on VEGF and a second epitope on HER2, wherein the antibody comprises an antigen-binding site comprising a light chain variable domain (V_{L}) comprising the following complementarity determining regions (CDRs):
(a) a CDR-L1 comprising the amino acid sequence of any one of SEQ ID NOs: 603-621,
(b) a CDR-L2 comprising the amino acid sequence of SEQ ID NO: 25 or 641, and
(c) a CDR-L3 comprising the amino acid sequence of SEQ ID NO: 622-631, and a heavy chain variable domain (V_{H}) comprising the following CDRs:
(d) a CDR-H1 comprising the amino acid sequence of SEQ ID NO: 1,
(e) a CDR-H1 comprising the amino acid sequence of SEQ ID NO: 2, and
(f) a CDR-H1 comprising the amino acid sequence of SEQ ID NO: 3, and
wherein the administering is for a time and in an amount sufficient to treat the tumor in the subject.

45. The method of claim 44, wherein the antibody is monoclonal antibody.

46. The method of claim 44, wherein the antibody is a Fab or a scFv fragment.

47. The method of claim 44, wherein the antibody is an IgG antibody.

48. The method of claim 44, wherein the antigen-binding site binds to the first epitope on VEGF and the second epitope on HER2 with at least 10⁻⁴ M affinity.

49. The method of claim 44, wherein the tumor is a pre-cancerous tumor.

50. The method of claim 44, wherein the tumor is a non-metastatic tumor.

51. The method of claim 44, wherein the tumor is a cancerous tumor.

52. The method of claim 44, wherein the administering results in a reduction in tumor size.

53. The method of claim 44, wherein the administering results in an inhibition of tumor growth.

54. The method of claim 44, wherein the administering results in an inhibition of tumor metastasis.

55. The method of claim 44, wherein the antibody is delivered systemically to the subject.

56. The method of claim 44, wherein the antibody is delivered locally to the subject.

57. The method of claim 44, wherein the antibody is delivered directly to the tumor.


SEQUENCE LISTING (631 sequences)

SEQ ID NO: 1 - protein, 6 aa, synthetic construct
  source 1..6: note = synthetic
IKDTYI

SEQ ID NO: 2 - protein, 12 aa, synthetic construct
  source 1..12: note = synthetic
ARIYPTNGYTRY

SEQ ID NO: 3 - protein, 8 aa, synthetic construct
  source 1..8: note = synthetic
RWGGDGFY

SEQ ID NO: 4 - protein, 6 aa, synthetic construct
  source 1..6: note = synthetic
DVSTAV

SEQ ID NO: 5 - protein, 4 aa, synthetic construct
  source 1..4: note = synthetic
SASF

SEQ ID NO: 6 - protein, 6 aa, synthetic construct
  source 1..6: note = synthetic
HYTTPP

SEQ ID NO: 7 - protein, 6 aa, synthetic construct
  source 1..6: note = synthetic
FTDYTM

SEQ ID NO: 8 - protein, 12 aa, synthetic construct
  source 1..12: note = synthetic
ADVNPNSGGSIY

SEQ ID NO: 9 - protein, 7 aa, synthetic construct
  source 1..7: note = synthetic
RNLGPSF

SEQ ID NO: 10 - protein, 6 aa, synthetic construct
  source 1..6: note = synthetic
DVSIGV

SEQ ID NO: 11 - protein, 4 aa, synthetic construct
  source 1..4: note = synthetic
SASY

SEQ ID NO: 12 - protein, 6 aa, synthetic construct
  source 1..6: note = synthetic
YYIYPY

SEQ ID NO: 13 - protein, 6 aa, synthetic construct
  source 1..6: note = synthetic
ISDSGI

SEQ ID NO: 14 - protein, 12 aa, synthetic construct
  source 1..12: note = synthetic
AAIAPGAGSTYY

SEQ ID NO: 15 - protein, 8 aa, synthetic construct
  source 1..8: note = synthetic
RFVSAPPS

SEQ ID NO: 16 - protein, 5 aa, synthetic construct
  source 1..5: note = synthetic
GSGTD

SEQ ID NO: 17 - skipped

SEQ ID NO: 18 - protein, 6 aa, synthetic construct
  source 1..6: note = synthetic
YSTVPW

SEQ ID NO: 19 - protein, 214 aa, synthetic construct
  source 1..214: note = synthetic
DIQMTQSPSSLSASVGDRVTITCRASQDVNTAVAWYQQKPGKAPKLLIYSASFLYSGVPSRFSGSGSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 227 aa, synthetic construct
  source 1..227: note = synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 21 - protein, 6 aa, synthetic construct
  source 1..6: note = synthetic
NVWDWV

SEQ ID NO: 22 - protein, 4 aa, synthetic construct
  source 1..4: note = synthetic
PASS

SEQ ID NO: 23 - protein, 5 aa, synthetic construct
  source 1..5: note = synthetic
GWYIA

SEQ ID NO: 24 - protein, 10 aa, synthetic construct
  source 1..10: note = synthetic
DIPRSISGYV

SEQ ID NO: 25 - protein, 4 aa, synthetic construct
  source 1..4: note = synthetic
WGSY

SEQ ID NO: 26 - protein, 4 aa, synthetic construct
  source 1..4: note = synthetic
HYTT

SEQ ID NO: 27 - protein, 7 aa, synthetic construct
  source 1..7: note = synthetic
DIGLGSV

SEQ ID NO: 28 - protein, 4 aa, synthetic construct
  source 1..4: note = synthetic
WASY

SEQ ID NO: 29 - protein, 7 aa, synthetic construct
  source 1..7: note = synthetic
DIRSGSV

SEQ ID NO: 30 - protein, 8 aa, synthetic construct
  source 1..8: note = synthetic
DIWNRRAL

SEQ ID NO: 31 - protein, 4 aa, synthetic construct
  source 1..4: note = synthetic
EGSS

SEQ ID NO: 32 - protein, 6 aa, synthetic construct
  source 1..6: note = synthetic
GGSYSS

SEQ ID NO: 33 - protein, 6 aa, synthetic construct
  source 1..6: note = synthetic
NVGRPV

SEQ ID NO: 34 - protein, 4 aa, synthetic construct
  source 1..4: note = synthetic
GGSS

SEQ ID NO: 35 - protein, 6 aa, synthetic construct
  source 1..6: note = synthetic
YGSFGT

SEQ ID NO: 36 - protein, 6 aa, synthetic construct
  source 1..6: note = synthetic
NVSKHV

SEQ ID NO: 37 - protein, 4 aa, synthetic construct
  source 1..4: note = synthetic
SYSG

SEQ ID NO: 38 - protein, 8 aa, synthetic construct
  source 1..8: note = synthetic
NIRNGGGL

SEQ ID NO: 39 - protein, 4 aa, synthetic construct
  source 1..4: note = synthetic
VNTA

SEQ ID NO: 40 - protein, 8 aa, synthetic construct
  source 1..8: note = synthetic
IPRSISGY

SEQ ID NO: 41-46 - skipped (6 entries)

SEQ ID NO: 47 - DNA, 12 bp, synthetic construct
  misc_difference 10..11: note = misc_feature - n is a, c, g, or t
  source 1..12: mol_type = other DNA; note = synthetic
tgggsttccnnk

SEQ ID NO: 48 - DNA, 12 bp, synthetic construct
  source 1..12: mol_type = other DNA; note = synthetic
kgggsttcctmt

SEQ ID NO: 49 - DNA, 12 bp, synthetic construct
  misc_difference 1..2: note = misc_feature - n is a, c, g, or t
  source 1..12: mol_type = other DNA; note = synthetic
nnkgsttcctmt

SEQ ID NO: 50 - DNA, 18 bp, synthetic construct
  misc_difference 1: note = misc_feature - n = 70% G or 10 % A or 10 % T or 10% C
  misc_difference 2: note = misc_feature - n = 70% A or 10 % G or 10 % T or 10% C
  misc_difference 3: note = misc_feature - n = 70% C or 10 % A or 10 % T or 10% G
  misc_difference 7..8: note = misc_feature - n is a, c, g, or t
  misc_difference 10..11: note = misc_feature - n is a, c, g, or t
  source 1..18: mol_type = other DNA; note = synthetic
nnnrttnnknnktacsta

SEQ ID NO: 51 - DNA, 18 bp, synthetic construct
  misc_difference 1: note = misc_feature - n = 70% G or 10 % A or 10 % T or 10% C
  misc_difference 2: note = misc_feature - n = 70% A or 10 % G or 10 % T or 10% C
  misc_difference 3: note = misc_feature - n = 70% C or 10 % G or 10 % T or 10% A
  misc_difference 7..8: note = misc_feature - n is a, c, g, or t
  misc_difference 10..11: note = misc_feature - n is a, c, g, or t
  source 1..18: mol_type = other DNA; note = synthetic
nnnrttnnknnkdggsta

SEQ ID NO: 52 - DNA, 18 bp, synthetic construct
  misc_difference 1: note = misc_feature - n = 70% G or 10 % A or 10 % T or 10% C
  misc_difference 2: note = misc_feature - n = 70% A or 10 % G or 10 % T or 10% C
  misc_difference 3: note = misc_feature - n = 70% C or 10 % G or 10 % T or 10% A
  misc_difference 7..8: note = misc_feature - n is a, c, g, or t
  misc_difference 10..11: note = misc_feature - n is a, c, g, or t
  misc_difference 13: note = misc_feature - n is a, c, g, or t
  source 1..18: mol_type = other DNA; note = synthetic
nnnrttnnknnknmtsta

SEQ ID NO: 53-63 - skipped (11 entries)

SEQ ID NO: 64 - protein, 7 aa, synthetic construct
  source 1..7: note = synthetic
DIRTGSV

SEQ ID NO: 65 - protein, 7 aa, synthetic construct
  source 1..7: note = synthetic
DIRVGSV

SEQ ID NO: 66 - protein, 7 aa, synthetic construct
  source 1..7: note = synthetic
DIAGGSV

SEQ ID NO: 67 - protein, 7 aa, synthetic construct
  source 1..7: note = synthetic
DIRFGSV

SEQ ID NO: 68 - protein, 7 aa, synthetic construct
  source 1..7: note = synthetic
DIKSGSV

SEQ ID NO: 69 - protein, 7 aa, synthetic construct
  source 1..7: note = synthetic
DIARGSV

SEQ ID NO: 70 - protein, 7 aa, synthetic construct
  source 1..7: note = synthetic
WVPSHTV

SEQ ID NO: 71 - protein, 7 aa, synthetic construct
  source 1..7: note = synthetic
DINGGSV

SEQ ID NO: 72 - protein, 7 aa, synthetic construct
  source 1..7: note = synthetic
DILQGSV

SEQ ID NO: 73 - protein, 7 aa, synthetic construct
  source 1..7: note = synthetic
DVRMGSV

SEQ ID NO: 74 - protein, 7 aa, synthetic construct
  source 1..7: note = synthetic
DIKHGSV

SEQ ID NO: 75 - protein, 7 aa, synthetic construct
  source 1..7: note = synthetic
DIASGSV

SEQ ID NO: 76 - protein, 7 aa, synthetic construct
  source 1..7: note = synthetic
DIKSGSL

SEQ ID NO: 77 - protein, 6 aa, synthetic construct
  source 1..6: note = synthetic
NVWDWV

SEQ ID NO: 78 - protein, 7 aa, synthetic construct
  source 1..7: note = synthetic
DIRMGSV

SEQ ID NO: 79 - protein, 6 aa, synthetic construct
  source 1..6: note = synthetic
QIWRHL

SEQ ID NO: 80 - protein, 7 aa, synthetic construct
  source 1..7: note = synthetic
DISHGSV

SEQ ID NO: 81 - protein, 7 aa, synthetic construct
  source 1..7: note = synthetic
NIAMGSV

SEQ ID NO: 82 - protein, 6 aa, synthetic construct
  source 1..6: note = synthetic
EVYSYV

SEQ ID NO: 83 - protein, 10 aa, synthetic construct
  source 1..10: note = synthetic
VIRKAFGQPL

SEQ ID NO: 84 - protein, 7 aa, synthetic construct
  source 1..7: note = synthetic
DIKKGSV

SEQ ID NO: 85 - protein, 6 aa, synthetic construct
  source 1..6: note = synthetic
NIFSHV

SEQ ID NO: 86 - protein, 7 aa, synthetic construct
  source 1..7: note = synthetic
DILFGSV

SEQ ID NO: 87 - protein, 6 aa, synthetic construct
  source 1..6: note = synthetic
DIWRWL

SEQ ID NO: 88 - protein, 7 aa, synthetic construct
  source 1..7: note = synthetic
DIYMGSV

SEQ ID NO: 89 - protein, 7 aa, synthetic construct
  source 1..7: note = synthetic
DISMGSV

SEQ ID NO: 90 - protein, 7 aa, synthetic construct
  source 1..7: note = synthetic
NIAQGSV

SEQ ID NO: 91 - protein, 4 aa, synthetic construct
  source 1..4: note = synthetic
WGSF

SEQ ID NO: 92 - protein, 4 aa, synthetic construct
  source 1..4: note = synthetic
LGSY

SEQ ID NO: 93 - protein, 4 aa, synthetic construct
  source 1..4: note = synthetic
WASF

SEQ ID NO: 94 - protein, 4 aa, synthetic construct
  source 1..4: note = synthetic
WGSS

SEQ ID NO: 95 - protein, 4 aa, synthetic construct
  source 1..4: note = synthetic
WGSV

SEQ ID NO: 96 - protein, 4 aa, synthetic construct
  source 1..4: note = synthetic
TGSS

SEQ ID NO: 97 - protein, 4 aa, synthetic construct
  source 1..4: note = synthetic
GASY

SEQ ID NO: 98 - protein, 4 aa, synthetic construct
  source 1..4: note = synthetic
PGSY

SEQ ID NO: 99 - protein, 4 aa, synthetic construct
  source 1..4: note = synthetic
AGSS

SEQ ID NO: 100-105 - skipped (6 entries)

SEQ ID NO: 106 - protein, 7 aa, synthetic construct
  source 1..7: note = synthetic
RINSHTV

SEQ ID NO: 107 - protein, 6 aa, synthetic construct
  source 1..6: note = synthetic
VVSMTV

SEQ ID NO: 108 - protein, 6 aa, synthetic construct
  source 1..6: note = synthetic
SVSSAV

SEQ ID NO: 109 - protein, 6 aa, synthetic construct
  source 1..6: note = synthetic
EVLTSV

SEQ ID NO: 110 - protein, 7 aa, synthetic construct
  source 1..7: note = synthetic
DVSRYDL

SEQ ID NO: 111 - protein, 6 aa, synthetic construct
  source 1..6: note = synthetic
EIVFSV

SEQ ID NO: 112 - protein, 7 aa, synthetic construct
  source 1..7: note = synthetic
AVPRYGV

SEQ ID NO: 113 - protein, 8 aa, synthetic construct
  source 1..8: note = synthetic
DIPEHFRL

SEQ ID NO: 114 - protein, 7 aa, synthetic construct
  source 1..7: note = synthetic
YIPRDAL

SEQ ID NO: 115 - protein, 6 aa, synthetic construct
  source 1..6: note = synthetic
DIGAGL

SEQ ID NO: 116 - protein, 7 aa, synthetic construct
  source 1..7: note = synthetic
DVHRDSV

SEQ ID NO: 117 - protein, 7 aa, synthetic construct
  source 1..7: note = synthetic
DVGGLGL

SEQ ID NO: 118 - protein, 6 aa, synthetic construct
  source 1..6: note = synthetic
DIFPRV

SEQ ID NO: 119 - protein, 7 aa, synthetic construct
  source 1..7: note = synthetic
AVSRHAL

SEQ ID NO: 120 - protein, 7 aa, synthetic construct
  source 1..7: note = synthetic
NVPRWSL

SEQ ID NO: 121 - protein, 7 aa, synthetic construct
  source 1..7: note = synthetic
PVFRRGL

SEQ ID NO: 122 - protein, 7 aa, synthetic construct
  source 1..7: note = synthetic
QVSKYDV

SEQ ID NO: 123 - protein, 7 aa, synthetic construct
  source 1..7: note = synthetic
VIARYDL

SEQ ID NO: 124 - protein, 10 aa, synthetic construct
  source 1..10: note = synthetic
QIVTPTAVHV

SEQ ID NO: 125 - protein, 8 aa, synthetic construct
  source 1..8: note = synthetic
DVSGRRGL

SEQ ID NO: 126 - protein, 6 aa, synthetic construct
  source 1..6: note = synthetic
DVNRYV

SEQ ID NO: 127 - protein, 11 aa, synthetic construct
  source 1..11: note = synthetic
NVSRVSWFETL

SEQ ID NO: 128 - protein, 7 aa, synthetic construct
  source 1..7: note = synthetic
EISRYAV

SEQ ID NO: 129 - protein, 7 aa, synthetic construct
  source 1..7: note = synthetic
NVPLNDV

SEQ ID NO: 130 - protein, 8 aa, synthetic construct
  source 1..8: note = synthetic
DVGGGSGV

SEQ ID NO: 131 - protein, 10 aa, synthetic construct
  source 1..10: note = synthetic
RVSDSLQNSV

SEQ ID NO: 132 - protein, 9 aa, synthetic construct
  source 1..9: note = synthetic
AVRRLTAYV

SEQ ID NO: 133 - protein, 6 aa, synthetic construct
  source 1..6: note = synthetic
DISRYL

SEQ ID NO: 134 - protein, 6 aa, synthetic construct
  source 1..6: note = synthetic
KIQAYV

SEQ ID NO: 135 - protein, 8 aa, synthetic construct
  source 1..8: note = synthetic
DVHPSPRV

SEQ ID NO: 136 - protein, 8 aa, synthetic construct
  source 1..8: note = synthetic
NIVVRPYV

SEQ ID NO: 137 - protein, 8 aa, synthetic construct
  source 1..8: note = synthetic
DIRGQRGV

SEQ ID NO: 138 - protein, 6 aa, synthetic construct
  source 1..6: note = synthetic
VVRQHV

SEQ ID NO: 139 - protein, 7 aa, synthetic construct
  source 1..7: note = synthetic
DIGLLNL

SEQ ID NO: 140 - protein, 4 aa, synthetic construct
  source 1..4: note = synthetic
WGSH

SEQ ID NO: 141 - protein, 4 aa, synthetic construct
  source 1..4: note = synthetic
KGSS

SEQ ID NO: 142 - protein, 4 aa, synthetic construct
  source 1..4: note = synthetic
GASS

SEQ ID NO: 143 - protein, 4 aa, synthetic construct
  source 1..4: note = synthetic
SASS

SEQ ID NO: 144 - protein, 4 aa, synthetic construct
  source 1..4: note = synthetic
FASS

SEQ ID NO: 145 - protein, 4 aa, synthetic construct
  source 1..4: note = synthetic
WGSD

SEQ ID NO: 146 - protein, 4 aa, synthetic construct
  source 1..4: note = synthetic
WGSQ

SEQ ID NO: 147 - protein, 4 aa, synthetic construct
  source 1..4: note = synthetic
AGSY

SEQ ID NO: 148 - protein, 4 aa, synthetic construct
  source 1..4: note = synthetic
WASS

SEQ ID NO: 149 - protein, 4 aa, synthetic construct
  source 1..4: note = synthetic
DASS

SEQ ID NO: 150 - protein, 4 aa, synthetic construct
  source 1..4: note = synthetic
GGSY

SEQ ID NO: 151 - protein, 4 aa, synthetic construct
  source 1..4: note = synthetic
HGSS

SEQ ID NO: 152 - protein, 4 aa, synthetic construct
  source 1..4: note = synthetic
LGSS

SEQ ID NO: 153 - protein, 4 aa, synthetic construct
  source 1..4: note = synthetic
SGSS

SEQ ID NO: 154 - protein, 4 aa, synthetic construct
  source 1..4: note = synthetic
RASS

SEQ ID NO: 155 - protein, 5 aa, synthetic construct
  source 1..5: note = synthetic
GGSYS

SEQ ID NO: 156 - protein, 5 aa, synthetic construct
  source 1..5: note = synthetic
YSNRT

SEQ ID NO: 157 - protein, 5 aa, synthetic construct
  source 1..5: note = synthetic
YGSYS

SEQ ID NO: 158 - protein, 5 aa, synthetic construct
  source 1..5: note = synthetic
YGSFG

SEQ ID NO: 159 - protein, 4 aa, synthetic construct
  source 1..4: note = synthetic
YSSS

SEQ ID NO: 160 - protein, 5 aa, synthetic construct
  source 1..5: note = synthetic
SGYHT

SEQ ID NO: 161 - protein, 5 aa, synthetic construct
  source 1..5: note = synthetic
GDSKS

SEQ ID NO: 162 - protein, 5 aa, synthetic construct
  source 1..5: note = synthetic
SGWSA

SEQ ID NO: 163 - protein, 6 aa, synthetic construct
  source 1..6: note = synthetic
GGWSET

SEQ ID NO: 164 - protein, 6 aa, synthetic construct
  source 1..6: note = synthetic
WASWDA

SEQ ID NO: 165 - protein, 5 aa, synthetic construct
  source 1..5: note = synthetic
SESRS

SEQ ID NO: 166 - protein, 6 aa, synthetic construct
  source 1..6: note = synthetic
WTWADT

SEQ ID NO: 167 - protein, 5 aa, synthetic construct
  source 1..5: note = synthetic
GGADT

SEQ ID NO: 168 - protein, 4 aa, synthetic construct
  source 1..4: note = synthetic
GHSG

SEQ ID NO: 169 - protein, 5 aa, synthetic construct
  source 1..5: note = synthetic
WDGKA

SEQ ID NO: 170 - protein, 6 aa, synthetic construct
  source 1..6: note = synthetic
WVTHET

SEQ ID NO: 171 - protein, 5 aa, synthetic construct
  source 1..5: note = synthetic
GLRHG

SEQ ID NO: 172 - protein, 5 aa, synthetic construct
  source 1..5: note = synthetic
SGFRS

SEQ ID NO: 173 - protein, 5 aa, synthetic construct
  source 1..5: note = synthetic
AGARA

SEQ ID NO: 174 - protein, 4 aa, synthetic construct
  source 1..4: note = synthetic
GTTT

SEQ ID NO: 175 - protein, 5 aa, synthetic construct
  source 1..5: note = synthetic
GAAYA

SEQ ID NO: 176 - protein, 5 aa, synthetic construct
  source 1..5: note = synthetic
GIDLA

SEQ ID NO: 177 - protein, 5 aa, synthetic construct
  source 1..5: note = synthetic
WFTWG

SEQ ID NO: 178 - protein, 5 aa, synthetic construct
  source 1..5: note = synthetic
GVYNA

SEQ ID NO: 179 - protein, 5 aa, synthetic construct
  source 1..5: note = synthetic
WAHYG

SEQ ID NO: 180 - protein, 5 aa, synthetic construct
  source 1..5: note = synthetic
GLDLA

SEQ ID NO: 181 - protein, 6 aa, synthetic construct
  source 1..6: note = synthetic
YGWRRT

SEQ ID NO: 182 - protein, 5 aa, synthetic construct
  source 1..5: note = synthetic
ANVDT

SEQ ID NO: 183 - protein, 6 aa, synthetic construct
  source 1..6: note = synthetic
GSGSST

SEQ ID NO: 184 - protein, 5 aa, synthetic construct
  source 1..5: note = synthetic
SDASA

SEQ ID NO: 185 - protein, 6 aa, synthetic construct
  source 1..6: note = synthetic
WADIST

SEQ ID NO: 186-216 - skipped (31 entries)

SEQ ID NO: 217 - protein, 6 aa, synthetic construct
  source 1..6: note = synthetic
YIWNYV

SEQ ID NO: 218 - protein, 5 aa, synthetic construct
  source 1..5: note = synthetic
SWWAG

SEQ ID NO: 219 - skipped

SEQ ID NO: 220 - protein, 7 aa, synthetic construct
  source 1..7: note = synthetic
EIFPYYV

SEQ ID NO: 221 - protein, 4 aa, synthetic construct
  source 1..4: note = synthetic
GWDG

SEQ ID NO: 222 - protein, 6 aa, synthetic construct
  source 1..6: note = synthetic
YVWQYV

SEQ ID NO: 223 - protein, 4 aa, synthetic construct
  source 1..4: note = synthetic
HASS

SEQ ID NO: 224 - protein, 5 aa, synthetic construct
  source 1..5: note = synthetic
GYWVA

SEQ ID NO: 225 - protein, 6 aa, synthetic construct
  source 1..6: note = synthetic
DVFTSV

SEQ ID NO: 226 - protein, 4 aa, synthetic construct
  source 1..4: note = synthetic
DASY

SEQ ID NO: 227 - protein, 5 aa, synthetic construct
  source 1..5: note = synthetic
RYIWA

SEQ ID NO: 228-231 - skipped (4 entries)

SEQ ID NO: 232 - protein, 7 aa, synthetic construct
  source 1..7: note = synthetic
DIMLGSV

SEQ ID NO: 233 - protein, 7 aa, synthetic construct
  source 1..7: note = synthetic
DIGLGSV

SEQ ID NO: 234 - protein, 7 aa, synthetic construct
  source 1..7: note = synthetic
DIRGGSV

SEQ ID NO: 235 - protein, 7 aa, synthetic construct
  source 1..7: note = synthetic
DIAKGSV

SEQ ID NO: 236 - protein, 7 aa, synthetic construct
  source 1..7: note = synthetic
DILAGSV

SEQ ID NO: 237 - protein, 7 aa, synthetic construct
  source 1..7: note = synthetic
DISRGSV

SEQ ID NO: 238 - protein, 7 aa, synthetic construct
  source 1..7: note = synthetic
NIYAGSL

SEQ ID NO: 239 - protein, 7 aa, synthetic construct
  source 1..7: note = synthetic
DIAFGSL

SEQ ID NO: 240 - protein, 7 aa, synthetic construct
  source 1..7: note = synthetic
DIKAGSV

SEQ ID NO: 241 - protein, 7 aa, synthetic construct
  source 1..7: note = synthetic
DILKGSV

SEQ ID NO: 242 - protein, 7 aa, synthetic construct
  source 1..7: note = synthetic
DILIGSV

SEQ ID NO: 243 - protein, 7 aa, synthetic construct
  source 1..7: note = synthetic
DIVSGSV

SEQ ID NO: 244 - skipped

SEQ ID NO: 245 - protein, 7 aa, synthetic construct
  source 1..7: note = synthetic
DIRQGSV

SEQ ID NO: 246 - protein, 7 aa, synthetic construct
  source 1..7: note = synthetic
DIKVGSV

SEQ ID NO: 247 - protein, 7 aa, synthetic construct
  source 1..7: note = synthetic
DIREGSV

SEQ ID NO: 248 - protein, 7 aa, synthetic construct
  source 1..7: note = synthetic
DIGSGSV

SEQ ID NO: 249 - protein, 7 aa, synthetic construct
  source 1..7: note = synthetic
DILTGSV

SEQ ID NO: 250 - protein, 7 aa, synthetic construct
  source 1..7: note = synthetic
NIRTGSV

SEQ ID NO: 251 - protein, 7 aa, synthetic construct
  source 1..7: note = synthetic
DISSGSV

SEQ ID NO: 252 - protein, 7 aa, synthetic construct
  source 1..7: note = synthetic
DISVGSV

SEQ ID NO: 253 - protein, 7 aa, synthetic construct
  source 1..7: note = synthetic
DVRQGSV

SEQ ID NO: 254 - protein, 7 aa, synthetic construct
  source 1..7: note = synthetic
DIQSGSV

SEQ ID NO: 255 - protein, 7 aa, synthetic construct
  source 1..7: note = synthetic
DIMSGSV

SEQ ID NO: 256 - protein, 7 aa, synthetic construct
  source 1..7: note = synthetic
DIIGGSV

SEQ ID NO: 257 - protein, 7 aa, synthetic construct
  source 1..7: note = synthetic
DILGGSV

SEQ ID NO: 258 - protein, 7 aa, synthetic construct
  source 1..7: note = synthetic
DIRLGSV

SEQ ID NO: 259 - protein, 7 aa, synthetic construct
  source 1..7: note = synthetic
DIGAGSV

SEQ ID NO: 260 - protein, 7 aa, synthetic construct
  source 1..7: note = synthetic
DIATGSL

SEQ ID NO: 261 - protein, 7 aa, synthetic construct
  source 1..7: note = synthetic
DILSGSV

SEQ ID NO: 262 - protein, 7 aa, synthetic construct
  source 1..7: note = synthetic
GIRTGSV

SEQ ID NO: 263 - protein, 7 aa, synthetic construct
  source 1..7: note = synthetic
DIAMGSV

SEQ ID NO: 264 - protein, 7 aa, synthetic construct
  source 1..7: note = synthetic
DIKLGSV

SEQ ID NO: 265 - protein, 7 aa, synthetic construct
  source 1..7: note = synthetic
NIYSGSL

SEQ ID NO: 266 - protein, 7 aa, synthetic construct
  source 1..7: note = synthetic
DIRAGSV

SEQ ID NO: 267 - protein, 7 aa, synthetic construct
  source 1..7: note = synthetic
DIAAGSV

SEQ ID NO: 268 - protein, 7 aa, synthetic construct
  source 1..7: note = synthetic
DIQTGSV

SEQ ID NO: 269 - protein, 7 aa, synthetic construct
  source 1..7: note = synthetic
DITMGSV

SEQ ID NO: 270 - protein, 7 aa, synthetic construct
  source 1..7: note = synthetic
DIGMGSV

SEQ ID NO: 271 - protein, 7 aa, synthetic construct
  source 1..7: note = synthetic
DIQRGSV

SEQ ID NO: 272 - protein, 7 aa, synthetic construct
  source 1..7: note = synthetic
DIGGGSV

SEQ ID NO: 273 - protein, 7 aa, synthetic construct
  source 1..7: note = synthetic
DIKFGSV

SEQ ID NO: 274 - protein, 7 aa, synthetic construct
  source 1..7: note = synthetic
DIRRGSV

SEQ ID NO: 275 - protein, 7 aa, synthetic construct
  source 1..7: note = synthetic
DIRIGSV

SEQ ID NO: 276 - protein, 7 aa, synthetic construct
  VARIANT 4: note = misc_feature - Xaa can be any naturally occurring amino acid
  source 1..7: note = synthetic
DIVXGSV

SEQ ID NO: 277 - protein, 7 aa, synthetic construct
  source 1..7: note = synthetic
DIAHGSV

SEQ ID NO: 278 - protein, 7 aa, synthetic construct
  source 1..7: note = synthetic
DIKNGSV

SEQ ID NO: 279 - protein, 7 aa, synthetic construct
  source 1..7: note = synthetic
DIKWGSV

SEQ ID NO: 280 - protein, 7 aa, synthetic construct
  source 1..7: note = synthetic
NIRSGSV

SEQ ID NO: 281 - protein, 6 aa, synthetic construct
  source 1..6: note = synthetic
DVWKWV

SEQ ID NO: 282 - protein, 6 aa, synthetic construct
  source 1..6: note = synthetic
DIWHWV

SEQ ID NO: 283 - protein, 4 aa, synthetic construct
  source 1..4: note = synthetic
WGSW

SEQ ID NO: 284 - protein, 4 aa, synthetic construct
  source 1..4: note = synthetic
WGSL

SEQ ID NO: 285 - protein, 4 aa, synthetic construct
  source 1..4: note = synthetic
WGSM

SEQ ID NO: 286 - protein, 4 aa, synthetic construct
  source 1..4: note = synthetic
WGST

SEQ ID NO: 287 - protein, 4 aa, synthetic construct
  source 1..4: note = synthetic
WGSC

SEQ ID NO: 288 - protein, 4 aa, synthetic construct
  source 1..4: note = synthetic
WGSN

SEQ ID NO: 289 - protein, 4 aa, synthetic construct
  source 1..4: note = synthetic
WGSE

SEQ ID NO: 290 - protein, 4 aa, synthetic construct
  source 1..4: note = synthetic
AASS

SEQ ID NO: 291 - protein, 4 aa, synthetic construct
  source 1..4: note = synthetic
WGSA

SEQ ID NO: 292 - protein, 4 aa, synthetic construct
  source 1..4: note = synthetic
HFNA

SEQ ID NO: 293-419 - skipped (127 entries)

SEQ ID NO: 420 - protein, 6 aa, synthetic construct
  source 1..6: note = synthetic
DIWRAV

SEQ ID NO: 421 - protein, 6 aa, synthetic construct
  source 1..6: note = synthetic
DIWRWV

SEQ ID NO: 422 - protein, 7 aa, synthetic construct
  source 1..7: note = synthetic
YVYSTTV

SEQ ID NO: 423 - protein, 7 aa, synthetic construct
  source 1..7: note = synthetic
DIHSGSV

SEQ ID NO: 424 - protein, 7 aa, synthetic construct
  source 1..7: note = synthetic
DIPSIYV

SEQ ID NO: 425 - protein, 6 aa, synthetic construct
  source 1..6: note = synthetic
NVWSHV

SEQ ID NO: 426 - protein, 5 aa, synthetic construct
  source 1..5: note = synthetic
IWRYV

SEQ ID NO: 427 - protein, 4 aa, synthetic construct
  source 1..4: note = synthetic
PASN

SEQ ID NO: 428 - protein, 4 aa, synthetic construct
  source 1..4: note = synthetic
NGSS

SEQ ID NO: 429 - protein, 5 aa, synthetic construct
  source 1..5: note = synthetic
GYYIA

SEQ ID NO: 430 - protein, 4 aa, synthetic construct
  source 1..4: note = synthetic
SDYT

SEQ ID NO: 431 - protein, 5 aa, synthetic construct
  source 1..5: note = synthetic
SYWVA

SEQ ID NO: 432 - protein, 4 aa, synthetic construct
  source 1..4: note = synthetic
HAGA

SEQ ID NO: 433 - protein, 5 aa, synthetic construct
  source 1..5: note = synthetic
AFWVS

SEQ ID NO: 434 - protein, 4 aa, synthetic construct
  source 1..4: note = synthetic
ASSA

SEQ ID NO: 435 - protein, 5 aa, synthetic construct
  source 1..5: note = synthetic
RYWVA

SEQ ID NO: 436 - protein, 5 aa, synthetic construct
  source 1..5: note = synthetic
RLWFT

SEQ ID NO: 437 - protein, 5 aa, synthetic construct
  source 1..5: note = synthetic
GFWIG

SEQ ID NO: 438 - protein, 4 aa, synthetic construct
  source 1..4: note = synthetic
HDQA

SEQ ID NO: 439 - protein, 5 aa, synthetic construct
  source 1..5: note = synthetic
GLWYS

SEQ ID NO: 440-498 - skipped (59 entries)

SEQ ID NO: 499 - protein, 7 aa, synthetic construct
  source 1..7: note = synthetic
AISHLGL

SEQ ID NO: 500 - protein, 7 aa, synthetic construct
  source 1..7: note = synthetic
DIHANTV

SEQ ID NO: 501 - protein, 6 aa, synthetic construct
  source 1..6: note = synthetic
DIGASV

SEQ ID NO: 502 - protein, 7 aa, synthetic construct
  source 1..7: note = synthetic
DVPAGAV

SEQ ID NO: 503 - protein, 7 aa, synthetic construct
  source 1..7: note = synthetic
EVASVSV

SEQ ID NO: 504-507 - skipped (4 entries)

SEQ ID NO: 508 - protein, 6 aa, synthetic construct
  source 1..6: note = synthetic
YIWRYV

SEQ ID NO: 509-531 - skipped (23 entries)

SEQ ID NO: 532 - protein, 6 aa, synthetic construct
  source 1..6: note = synthetic
GGSYSS

SEQ ID NO: 533 - protein, 6 aa, synthetic construct
  source 1..6: note = synthetic
YGSYSA

SEQ ID NO: 534 - protein, 6 aa, synthetic construct
  source 1..6: note = synthetic
YGSFGT

SEQ ID NO: 535-589 - skipped (55 entries)

SEQ ID NO: 590 - protein, 9 aa, synthetic construct
  source 1..9: note = synthetic
DIPRSISGY

SEQ ID NO: 591 - protein, 10 aa, synthetic construct
  source 1..10: note = synthetic
QDIPRSISGY

SEQ ID NO: 592 - protein, 4 aa, synthetic construct
  source 1..4: note = synthetic
KDTY

SEQ ID NO: 593 - protein, 6 aa, synthetic construct
  source 1..6: note = synthetic
RYTNYR

SEQ ID NO: 594 - protein, 7 aa, synthetic construct
  source 1..7: note = synthetic
WGGDGFY

SEQ ID NO: 595-601 - skipped (7 entries)

SEQ ID NO: 602 - protein, 16 aa, synthetic construct
  source 1..16: note = synthetic
CRAIQDIPKTITGYVA

SEQ ID NO: 603 - protein, 16 aa, synthetic construct
  source 1..16: note = synthetic
CRAIQNIAKTITGYVA

SEQ ID NO: 604 - protein, 16 aa, synthetic construct
  source 1..16: note = synthetic
CRAVQNLPKTITGYVA

SEQ ID NO: 605 - protein, 16 aa, synthetic construct
  source 1..16: note = synthetic
CRAIEDLPKTVSGYVA

SEQ ID NO: 606 - protein, 16 aa, synthetic construct
  source 1..16: note = synthetic
CRAIQKVPRTITGYVA

SEQ ID NO: 607 - protein, 16 aa, synthetic construct
  source 1..16: note = synthetic
CRASVDLPKSVSGYVA

SEQ ID NO: 608 - protein, 16 aa, synthetic construct
  source 1..16: note = synthetic
CRASEDIPKTVSGYVA

SEQ ID NO: 609 - protein, 16 aa, synthetic construct
  source 1..16: note = synthetic
CRAIENLPKTISGYVA

SEQ ID NO: 610 - protein, 16 aa, synthetic construct
  source 1..16: note = synthetic
CRASQKIARTISGYVA

SEQ ID NO: 611 - protein, 16 aa, synthetic construct
  source 1..16: note = synthetic
CRASEDIPKSISGYVA

SEQ ID NO: 612 - protein, 16 aa, synthetic construct
  source 1..16: note = synthetic
CRASQDLPKSISGYVA

SEQ ID NO: 613 - protein, 16 aa, synthetic construct
  source 1..16: note = synthetic
CRAIENLPKSITGYVA

SEQ ID NO: 614 - protein, 16 aa, synthetic construct
  source 1..16: note = synthetic
CRAIENLPKSVSGYVA

SEQ ID NO: 615 - protein, 16 aa, synthetic construct
  source 1..16: note = synthetic
CRAVEDLPKTITGYVA

SEQ ID NO: 616 - protein, 16 aa, synthetic construct
  source 1..16: note = synthetic
CRASQEIAKTISGYVA

SEQ ID NO: 617 - protein, 16 aa, synthetic construct
  source 1..16: note = synthetic
CRASQNIPKSVSGYVA

SEQ ID NO: 618 - protein, 16 aa, synthetic construct
  source 1..16: note = synthetic
CRASEDLPKSVSGYVA

SEQ ID NO: 619 - protein, 16 aa, synthetic construct
  source 1..16: note = synthetic
CRASENIPRTVSGYVA

SEQ ID NO: 620 - protein, 16 aa, synthetic construct
  source 1..16: note = synthetic
CRASQDIPRSISGYVA

SEQ ID NO: 621 - protein, 7 aa, synthetic construct
  source 1..7: note = synthetic
HYTTPPT

SEQ ID NO: 622 - protein, 7 aa, synthetic construct
  source 1..7: note = synthetic
HFNSPPK

SEQ ID NO: 623 - protein, 7 aa, synthetic construct
  source 1..7: note = synthetic
HYTIPPK

SEQ ID NO: 624 - protein, 7 aa, synthetic construct
  source 1..7: note = synthetic
HYSSPPT

SEQ ID NO: 625 - protein, 7 aa, synthetic construct
  source 1..7: note = synthetic
HYTSPPT

SEQ ID NO: 626 - protein, 7 aa, synthetic construct
  source 1..7: note = synthetic
HFTSPPT

SEQ ID NO: 627 - protein, 7 aa, synthetic construct
  source 1..7: note = synthetic
HFSSPPT

SEQ ID NO: 628 - protein, 7 aa, synthetic construct
  source 1..7: note = synthetic
HYSTTPK

SEQ ID NO: 629 - protein, 7 aa, synthetic construct
  source 1..7: note = synthetic
HFTTPPK

SEQ ID NO: 630 - protein, 7 aa, synthetic construct
  source 1..7: note = synthetic
HFNSPPT

SEQ ID NO: 631 - skipped